SEQUENCE LISTING (80 sequences)

SEQ ID NO: 1 - protein, 18 aa, Artificial Sequence
  synthetic peptide
MKLCILLAVVAFVGLSLG

SEQ ID NO: 2 - protein, 29 aa, Artificial Sequence
  synthetic peptide
GSGYIPEAPRDGQAYVRKDGEWVLLSTFL

SEQ ID NO: 3 - DNA, 345 bp, Artificial Sequence
  synthetic construct
  misc_feature: anti-CD83 antibody clone F890/GE8 heavy chain
gaggtccagctgcaacaatctggacctgagctggtgaagcctggggcttcagtgaagatatcctgtaaggcttctggatacacgttcactgactactacataaactgggtgaagcagagccatggaaagagccttgagtggattggagatattaatcctactaatggtgattctacctacagccagaagttcaagggcaaggccacattgactgtagacaagtcctccagcacagcctacatggagctccgcagcctgacatctgaggtctctgcagtctattactgtgcaagagactatgctatggactactggggtcaaggaacctcagtcaccgtctcctca

SEQ ID NO: 4 - protein, 115 aa, Artificial Sequence
  synthetic polypeptide
  MISC_FEATURE: anti-CD83 antibody clone F890/GE8 heavy chain amino acid sequence
EVQLQQSGPELVKPGASVKISCKASGYTFTDYYINWVKQSHGKSLEWIGDINPTNGDSTYSQKFKGKATLTVDKSSSTAYMELRSLTSEVSAVYYCARDYAMDYWGQGTSVTVSS

SEQ ID NO: 5 - protein, 5 aa, Artificial Sequence
  synthetic peptide
  MISC_FEATURE: anti-CD83 antibody clone F890/GE8 heavy chain amino acid CDR-H1
DYYIN

SEQ ID NO: 6 - protein, 17 aa, Artificial Sequence
  synthetic peptide
  MISC_FEATURE: anti-CD83 antibody clone F890/GE8 heavy chain amino acid CDR-H2
DINPTNGDSTYSQKFKG

SEQ ID NO: 7 - protein, 6 aa, Artificial Sequence
  synthetic peptide
  MISC_FEATURE: anti-CD83 antibody clone F890/GE8 heavy chain amino acid CDR-H3
DYAMDY

SEQ ID NO: 8 - DNA, 339 bp, Artificial Sequence
  synthetic construct
  misc_feature: anti-CD83 antibody clone F890/GE8 light chain nucleotide sequence
gacattgtgatgacccagtctccatcctccctggctgtgtcagtcggacagaaggtcactatgagctgcacgtccagtcaggtccttttacatagtcccaatcaaaagaactatttggcctggtaccagcagaaaccaggacagtctcctaaacttctggtatactttgcatccactagggaatctggggtccctgatcgcttcacaggcagtggatctgggacagatttcactcttaccatcagcagtgtgcaggctgaagacctggcagtttattactgtcagcaacattatagcactccgctcacgttcggtgctgggaccaagctggagctgaaa

SEQ ID NO: 9 - protein, 113 aa, Artificial Sequence
  synthetic polypeptide
  MISC_FEATURE: anti-CD83 antibody clone F890/GE8 light chain amino acid sequence
DIVMTQSPSSLAVSVGQKVTMSCTSSQVLLHSPNQKNYLAWYQQKPGQSPKLLVYFASTRESGVPDRFTGSGSGTDFTLTISSVQAEDLAVYYCQQHYSTPLTFGAGTKLELK

SEQ ID NO: 10 - protein, 17 aa, Artificial Sequence
  synthetic peptide
  MISC_FEATURE: anti-CD83 antibody clone F890/GE8 light chain amino acid CDR-L1
TSSQVLLHSPNQKNYLA

SEQ ID NO: 11 - protein, 7 aa, Artificial Sequence
  synthetic peptide
  MISC_FEATURE: anti-CD83 antibody clone F890/GE8 light chain amino acid CDR-L2
FASTRES

SEQ ID NO: 12 - protein, 9 aa, Artificial Sequence
  synthetic peptide
  MISC_FEATURE: anti-CD83 antibody clone F890/GE8 light chain amino acid CDR-L3
QQHYSTPLT

SEQ ID NO: 13 - DNA, 360 bp, Artificial Sequence
  synthetic construct
  misc_feature: anti-CD11c antibody clone 8F2 heavy chain nucleotide sequence
gaggtccagctgcagcagtctggacctgagctggtaaagcctggggcttcagtgaagatgtcctgcaaggcttctggatacacattcactaactatgttctgcactgggtgaagcagaagcctgggcagggccttgagtggattggatatattaatccttacaatgatggtactaagttcaatgagaagttcaaaggcaaggccacactgacttcagacacatcctccagcacagccttcatggaactcagcagcctgacctctgaggactctgcggtctattactgtgcaagaggagataatctacggccctactactttgactactggggccaaggcaccactctcacagtctcctca

SEQ ID NO: 14 - protein, 120 aa, Artificial Sequence
  synthetic polypeptide
  MISC_FEATURE: anti-CD11c antibody clone 8F2 heavy chain amino acid sequence
EVQLQQSGPELVKPGASVKMSCKASGYTFTNYVLHWVKQKPGQGLEWIGYINPYNDGTKFNEKFKGKATLTSDTSSSTAFMELSSLTSEDSAVYYCARGDNLRPYYFDYWGQGTTLTVSS

SEQ ID NO: 15 - protein, 5 aa, Artificial Sequence
  synthetic peptide
  MISC_FEATURE: anti-CD11c antibody clone 8F2 heavy chain amino acid CDR-H1
NYVLH

SEQ ID NO: 16 - protein, 17 aa, Artificial Sequence
  synthetic peptide
  MISC_FEATURE: anti-CD11c antibody clone 8F2 heavy chain amino acid CDR-H2
YINPYNDGTKFNEKFKG

SEQ ID NO: 17 - protein, 11 aa, Artificial Sequence
  synthetic peptide
  MISC_FEATURE: anti-CD11c antibody clone 8F2 heavy chain amino acid CDR-H3
GDNLRPYYFDY

SEQ ID NO: 18 - DNA, 318 bp, Artificial Sequence
  synthetic construct
  misc_feature: anti-CD11c antibody clone 8F2 light chain nucleotide sequence
caaattgttctcacccattctccagcaatcatgtctgcatctccaggggagaaggtcaccatgacctgcagtgccagctcaagtgtaagtttcatgtactggtaccagcagaagccaggatcctccccccgactcctgctttatgacacatccagcctgtcttctggagtccctgttcgcttcagtggcagtggctctgggacctcttactctctcacaatcagccgaatggaggctgaagatgctgccacttattactgccagcagtggagtcgttacccaccgacgttcggtggaggcaccaagctggaaatcaaa

SEQ ID NO: 19 - protein, 106 aa, Artificial Sequence
  synthetic polypeptide
  MISC_FEATURE: anti-CD11c antibody clone 8F2 heavy chain amino acid sequence
QIVLTHSPAIMSASPGEKVTMTCSASSSVSFMYWYQQKPGSSPRLLLYDTSSLSSGVPVRFSGSGSGTSYSLTISRMEAEDAATYYCQQWSRYPPTFGGGTKLEIK

SEQ ID NO: 20 - protein, 10 aa, Artificial Sequence
  synthetic peptide
  MISC_FEATURE: anti-CD11c antibody clone 8F2 heavy chain amino acid CDR-L1
SASSSVSFMY

SEQ ID NO: 21 - protein, 7 aa, Artificial Sequence
  synthetic peptide
  MISC_FEATURE: anti-CD11c antibody clone 8F2 heavy chain amino acid CDR-L2
DTSSLSS

SEQ ID NO: 22 - protein, 9 aa, Artificial Sequence
  synthetic peptide
  MISC_FEATURE: anti-CD11c antibody clone 8F2 heavy chain amino acid CDR-L3
QQWSRYPPT

SEQ ID NO: 23 - DNA, 357 bp, Artificial Sequence
  synthetic construct
  misc_feature: anti-DEC205 antibody clone F887/AD6 heavy chain nucleotide sequence
gaggtgcaactggtggagtctgggggagacttagtgaagcctggagggtccctgaaactctcctgtgcagcctctggattcactttcagtagctatggcatgtcttgggttcgccagactccagacaagaggctggagtgggtcgcaaccattagtagtggtggtagttacacctactatccagacagtgtgaaggggcgattcaccatttccagagacaatgccaagaacatcctgtatctgcaaatgagcagtctgaagtctgaagacacagccatgtattactgtgcaagactttcaacctgggactggtacttcgatgtctggggcacagggaccacggtcaccgtctcctca

SEQ ID NO: 24 - protein, 119 aa, Artificial Sequence
  synthetic polypeptide
  MISC_FEATURE: anti-DEC205 antibody clone F887/AD6 heavy chain amino acid sequence
EVQLVESGGDLVKPGGSLKLSCAASGFTFSSYGMSWVRQTPDKRLEWVATISSGGSYTYYPDSVKGRFTISRDNAKNILYLQMSSLKSEDTAMYYCARLSTWDWYFDVWGTGTTVTVSS

SEQ ID NO: 25 - protein, 5 aa, Artificial Sequence
  synthetic peptide
  MISC_FEATURE: anti-DEC205 antibody clone F887/AD6 heavy chain amino acid CDR-H1
SYGMS

SEQ ID NO: 26 - protein, 17 aa, Artificial Sequence
  synthetic peptide
  MISC_FEATURE: anti-DEC205 antibody clone F887/AD6 heavy chain amino acid CDR-H2
SSGGSYTYYPDSVKGRF

SEQ ID NO: 27 - protein, 10 aa, Artificial Sequence
  synthetic peptide
  MISC_FEATURE: anti-DEC205 antibody clone F887/AD6 heavy chain amino acid CDR-H3
LSTWDWYFDV

SEQ ID NO: 28 - DNA, 324 bp, Artificial Sequence
  synthetic construct
  misc_feature: anti-DEC205 antibody clone F887/AD6 light chain nucleotide sequence
gaaattgtgctcacccagtctccagcactcatggctgcatctccaggggagaaggtcaccatcacctgcagtgtcagctcaagtataagttccggcaactttcactggtaccagcagaagtcaggaacctcccccaaactctggatttatggcacatccaacctggcttctggagtccctgttcgcttcagtggcagtggatctgggacctcttattctctcacaatcagcagcatggaggctgaagatgctgccacttattactgtcaacagtggagtagttacccattcacgttcggctcggggacaaagttggaaataaaa

SEQ ID NO: 29 - protein, 108 aa, Artificial Sequence
  synthetic polypeptide
  MISC_FEATURE: anti-DEC205 antibody clone F887/AD6 light chain amino acid sequence
EIVLTQSPALMAASPGEKVTITCSVSSSISSGNFHWYQQKSGTSPKLWIYGTSNLASGVPVRFSGSGSGTSYSLTISSMEAEDAATYYCQQWSSYPFTFGSGTKLEIK

SEQ ID NO: 30 - protein, 12 aa, Artificial Sequence
  synthetic peptide
  MISC_FEATURE: anti-DEC205 antibody clone F887/AD6 amino acid CDR-L1
SVSSSISSGNFH

SEQ ID NO: 31 - protein, 7 aa, Artificial Sequence
  synthetic peptide
  MISC_FEATURE: anti-DEC205 antibody clone F887/AD6 amino acid CDR-L2
GTSNLAS

SEQ ID NO: 32 - protein, 9 aa, Artificial Sequence
  synthetic peptide
  MISC_FEATURE: anti-DEC205 antibody clone F887/AD6 amino acid CDR-L3
QQWSSYPFT

SEQ ID NO: 33 - protein, 20 aa, Artificial Sequence
  synthetic peptide
  MISC_FEATURE: Cleavable linker
LEAGCKNFFPRSFTSCGSLE

SEQ ID NO: 34 - protein, 6 aa, Artificial Sequence
  synthetic peptide
GGSGGS

SEQ ID NO: 35 - protein, 7 aa, Artificial Sequence
  synthetic peptide
SGSGSGS

SEQ ID NO: 36 - protein, 10 aa, Artificial Sequence
  synthetic peptide
GGGGSGGGGS

SEQ ID NO: 37 - protein, 10 aa, Artificial Sequence
  synthetic peptide
GSGSGSGSGS

SEQ ID NO: 38 - protein, 12 aa, Artificial Sequence
  synthetic peptide
GGSGGSGGSGGS

SEQ ID NO: 39 - protein, 15 aa, Artificial Sequence
  synthetic peptide
GGGGSGGGGSGGGGS

SEQ ID NO: 40 - protein, 17 aa, Artificial Sequence
  synthetic peptide
MPLLLLLPLLWAGALAM

SEQ ID NO: 41 - protein, 491 aa, Artificial Sequence
  synthetic polypeptide
  MISC_FEATURE: H9HA ectodomain
DKICIGHQSTNSTETVDTLTETNVPVTHAKELLHTEHNGMLCATNLGHPLILDTCTIEGLIYGNPSCDLLLGGREWSYIVERPSAVNGTCYPGNVENLEELRTLFSSSSSYQRIQIFPDTIWNVTYTGTSKSCSDSFYRNMRWLTQKSGLYPVQDAQYTNNRGKDILFVWGIHHPPTDTAQTNLYTRTDTTTSVTTENLDRTFKPVIGPRPLVNGLIGRINYYWSVLKPGQTLRVRSNGNLIAPWYGHVLSGESHGRILKTDLNSGNCVVQCQTEKGGLNSTLPFHNISKYAFGNCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYKEESRLERQ

SEQ ID NO: 42 - DNA, 348 bp, Artificial Sequence
  synthetic construct
  misc_feature: anti-hemagglutinin antibody clone F955/IG10 heavy chain nucleotide sequence
gaggttcagctgcagcagtctgtggcagagcttgtgaggccaggggcctcagtcaagttgtcctgcacagcttctggcttcaacattaaaaacacctatatgcactgggtgaagcagaggcctgaacagggcctggagtggattggaaggattgatcctgcgaatggtaatactaggtatgccccgaagttccagggcaaggccactataactgcagacacatcctccaacacagcctacctgcagctcagcagcctgacatctgaggacactgccatctattactgtgcccgttattacttcggtcctgactactggggccaaggcaccactctcacagtctcctca

SEQ ID NO: 43 - protein, 116 aa, Artificial Sequence
  synthetic polypeptide
  MISC_FEATURE: anti-hemagglutinin antibody clone F955/IG10 heavy chain amino acid sequence
EVQLQQSVAELVRPGASVKLSCTASGFNIKNTYMHWVKQRPEQGLEWIGRIDPANGNTRYAPKFQGKATITADTSSNTAYLQLSSLTSEDTAIYYCARYYFGPDYWGQGTTLTVSS

SEQ ID NO: 44 - protein, 5 aa, Artificial Sequence
  synthetic peptide
  MISC_FEATURE: anti-hemagglutinin antibody clone F955/IG10 heavy chain amino acid CDR-H1
NTYMH

SEQ ID NO: 45 - protein, 17 aa, Artificial Sequence
  synthetic peptide
  MISC_FEATURE: anti-hemagglutinin antibody clone F955/IG10 heavy chain amino acid CDR-H2
RIDPANGNTRYAPKFQG

SEQ ID NO: 46 - protein, 7 aa, Artificial Sequence
  synthetic peptide
  MISC_FEATURE: anti-hemagglutinin antibody clone F955/IG10 heavy chain amino acid CDR-H3
YYFGPDY

SEQ ID NO: 47 - DNA, 321 bp, Artificial Sequence
  synthetic construct
  misc_feature: anti-hemagglutinin antibody clone F955/IG10 light chain nucleotide sequence
gacatcctgatgacccaatctccatcctccatgtctgtatctctgggagacacagtcatcatcacttgccatgcaagtcagggcattagcagtaatatagggtggttgcagcagaaaccagggaaatcatttaagggcctgatctatcatgcaaccaacttggaagatggagttccatcaaggttcagtggcggtggatctggagcagattattctctcaccatcagcagcctggaatctgaagattttgcagactattactgtgtacagtatggtcagtttccattcacgttcggctcggggacaaagttggaaataaaa

SEQ ID NO: 48 - protein, 107 aa, Artificial Sequence
  synthetic polypetide
  MISC_FEATURE: anti-hemagglutinin antibody clone F955/IG10 light chain amino acid sequence
DILMTQSPSSMSVSLGDTVIITCHASQGISSNIGWLQQKPGKSFKGLIYHATNLEDGVPSRFSGGGSGADYSLTISSLESEDFADYYCVQYGQFPFTFGSGTKLEIK

SEQ ID NO: 49 - protein, 11 aa, Artificial Sequence
  synthetic peptide
  MISC_FEATURE: anti-hemagglutinin antibody clone F955/IG10 light chain amino acid CDR-L1
HASQGISSNIG

SEQ ID NO: 50 - protein, 7 aa, Artificial Sequence
  synthetic peptide
  MISC_FEATURE: anti-hemagglutinin antibody clone F955/IG10 light chain amino acid CDR-L2
HATNLED

SEQ ID NO: 51 - protein, 9 aa, Artificial Sequence
  synthetic peptide
  MISC_FEATURE: anti-hemagglutinin antibody clone F955/IG10 light chain amino acid CDR-L3
VQYGQFPFT

SEQ ID NO: 52 - DNA, 354 bp, Artificial Sequence
  synthetic construct
  misc_feature: anti-hemagglutinin antibody clone F955/HD8 heavy chain nucleotide sequence
gaggttcagctgcagcagtctgtggcagagcttgtgaggccaggggcctcagtcaagttgtcctgcacagcttctggcttcaacattaaaaacacctatatgcactgggtgaagcagaggcctgaacagggcctggagtggattggaaggattgatcctgcgaatggtaatactagatatgccccgaaattccagggcaaggccactataactgcagacacatcctccaacacagcctacctgcagctcagcagcctgacatctgacgacactgccatctattactgtggtaggacagagttcaggaatgctatggactactggggtcaaggaacctcagtcaccgtctcctca

SEQ ID NO: 53 - protein, 118 aa, Artificial Sequence
  synthetic polypeptide
  MISC_FEATURE: anti-hemagglutinin antibody clone F955/HD8 heavy chain amino acid sequence
EVQLQQSVAELVRPGASVKLSCTASGFNIKNTYMHWVKQRPEQGLEWIGRIDPANGNTRYAPKFQGKATITADTSSNTAYLQLSSLTSDDTAIYYCGRTEFRNAMDYWGQGTSVTVSS

SEQ ID NO: 54 - protein, 5 aa, Artificial Sequence
  synthetic peptide
  MISC_FEATURE: anti-hemagglutinin antibody clone F955/HD8 heavy chain amino acid CDR-H1
NTYMH

SEQ ID NO: 55 - protein, 17 aa, Artificial Sequence
  synthetic peptide
  MISC_FEATURE: anti-hemagglutinin antibody clone F955/HD8 heavy chain amino acid CDR-H2
RIDPANGNTRYAPKFQG

SEQ ID NO: 56 - protein, 9 aa, Artificial Sequence
  synthetic peptide
  MISC_FEATURE: anti-hemagglutinin antibody clone F955/HD8 heavy chain amino acid CDR-H3
TEFRNAMDY

SEQ ID NO: 57 - DNA, 321 bp, Artificial Sequence
  synthetic construct
  misc_feature: anti-hemagglutinin antibody clone F955/HD8 light chain nucleotide sequence
gacatccagatgactcagtctccagcctccctatctccatctgtgggagaaactgtcaccatgacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagttttctctgaagatcaacagcctgcagcctgaagattttgggaattattactgtcaacatttttataatactccgtacacgttcggaggggggaccaagctggaaataaaa

SEQ ID NO: 58 - protein, 107 aa, Artificial Sequence
  synthetic polypeptide
  MISC_FEATURE: anti-hemagglutinin antibody clone F955/HD8 light chain amino acid sequence
DIQMTQSPASLSPSVGETVTMTCRASENIYSNLAWYQQKQGKSPQLLVYAATNLADGVPSRFSGSGSGTQFSLKINSLQPEDFGNYYCQHFYNTPYTFGGGTKLEIK

SEQ ID NO: 59 - protein, 11 aa, Artificial Sequence
  synthetic peptide
  MISC_FEATURE: anti-hemagglutinin antibody clone F955/HD8 light chain amino acid CDR-L1
RASENIYSNLA

SEQ ID NO: 60 - protein, 7 aa, Artificial Sequence
  synthetic peptide
  MISC_FEATURE: anti-hemagglutinin antibody clone F955/HD8 light chain amino acid CDR-L2
AATNLAD

SEQ ID NO: 61 - protein, 9 aa, Artificial Sequence
  synthetic peptide
  MISC_FEATURE: anti-hemagglutinin antibody clone F955/HD8 light chain amino acid CDR-L3
QHFYNTPYT

SEQ ID NO: 62 - protein, 811 aa, Artificial Sequence
  synthetic polypeptide
  MISC_FEATURE: BIP signal-H9HA ectodomain-Foldon-LINKER-CD83 scFv-V5-His tag
MKLCILLAVVAFVGLSLGDKICIGHQSTNSTETVDTLTETNVPVTHAKELLHTEHNGMLCATNLGHPLILDTCTIEGLIYGNPSCDLLLGGREWSYIVERPSAVNGTCYPGNVENLEELRTLFSSSSSYQRIQIFPDTIWNVTYTGTSKSCSDSFYRNMRWLTQKSGLYPVQDAQYTNNRGKDILFVWGIHHPPTDTAQTNLYTRTDTTTSVTTENLDRTFKPVIGPRPLVNGLIGRINYYWSVLKPGQTLRVRSNGNLIAPWYGHVLSGESHGRILKTDLNSGNCVVQCQTEKGGLNSTLPFHNISKYAFGNCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYKEESRLERQGSGYIPEAPRDGQAYVRKDGEWVLLSTFLGSGSGDIVMTQSPSSLAVSVGQKVTMSCTSSQVLLHSPNQKNYLAWYQQKPGQSPKLLVYFASTRESGVPDRFTGSGSGTDFTLTISSVQAEDLAVYYCQQHYSTPLTFGAGTKLELKGGGGSGGGGSGGGGSGGGGSEVQLQQSGPELVKPGASVKISCKASGYTFTDYYINWVKQSHGKSLEWIGDINPTNGDSTYSQKFKGKATLTVDKSSSTAYMELRSLTSEVSAVYYCARDYAMDYWGQGTSVTVSSGKPIPNPLLGLDSTHHHHHH

SEQ ID NO: 63 - protein, 809 aa, Artificial Sequence
  synthetic polypeptide
  MISC_FEATURE: BIP signal-H9HA ectodomain-Foldon-LINKER-CD11c scFv-V5-His tag
MKLCILLAVVAFVGLSLGDKICIGHQSTNSTETVDTLTETNVPVTHAKELLHTEHNGMLCATNLGHPLILDTCTIEGLIYGNPSCDLLLGGREWSYIVERPSAVNGTCYPGNVENLEELRTLFSSSSSYQRIQIFPDTIWNVTYTGTSKSCSDSFYRNMRWLTQKSGLYPVQDAQYTNNRGKDILFVWGIHHPPTDTAQTNLYTRTDTTTSVTTENLDRTFKPVIGPRPLVNGLIGRINYYWSVLKPGQTLRVRSNGNLIAPWYGHVLSGESHGRILKTDLNSGNCVVQCQTEKGGLNSTLPFHNISKYAFGNCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYKEESRLERQGSGYIPEAPRDGQAYVRKDGEWVLLSTFLGSGSGEVQLQQSGPELVKPGASVKMSCKASGYTFTNYVLHWVKQKPGQGLEWIGYINPYNDGTKFNEKFKGKATLTSDTSSSTAFMELSSLTSEDSAVYYCARGDNLRPYYFDYWGQGTTLTVSSGGGGSGGGGSGGGGSGGGGSQIVLTHSPAIMSASPGEKVTMTCSASSSVSFMYWYQQKPGSSPRLLLYDTSSLSSGVPVRFSGSGSGTSYSLTISRMEAEDAATYYCQQWSRYPPTFGGGTKLEIKGKPIPNPLLGLDSTHHHHHH

SEQ ID NO: 64 - protein, 810 aa, Artificial Sequence
  synthetic polypeptide
  MISC_FEATURE: BIP signal-H9HA ectodomain-Foldon-LINKER-Dec205 scFv-V5-His tag
MKLCILLAVVAFVGLSLGDKICIGHQSTNSTETVDTLTETNVPVTHAKELLHTEHNGMLCATNLGHPLILDTCTIEGLIYGNPSCDLLLGGREWSYIVERPSAVNGTCYPGNVENLEELRTLFSSSSSYQRIQIFPDTIWNVTYTGTSKSCSDSFYRNMRWLTQKSGLYPVQDAQYTNNRGKDILFVWGIHHPPTDTAQTNLYTRTDTTTSVTTENLDRTFKPVIGPRPLVNGLIGRINYYWSVLKPGQTLRVRSNGNLIAPWYGHVLSGESHGRILKTDLNSGNCVVQCQTEKGGLNSTLPFHNISKYAFGNCPKYIGVKSLKLAIGLRNVPARSSRGLFGAIAGFIEGGWPGLVAGWYGFQHSNDQGVGMAADRDSTQKAVDKITSKVNNIVDKMNKQYEIIDHEFSEVETRLNMINNKIDDQIQDVWAYNAELLVLLENQKTLDEHDANVNNLYNKVKRALGSNAMEDGKGCFELYHKCDDQCMETIRNGTYNRRKYKEESRLERQGSGYIPEAPRDGQAYVRKDGEWVLLSTFLGSGSGEIVLTQSPALMAASPGEKVTITCSVSSSISSGNFHWYQQKSGTSPKLWIYGTSNLASGVPVRFSGSGSGTSYSLTISSMEAEDAATYYCQQWSSYPFTFGSGTKLEIKGGGGSGGGGSGGGGSGGGGSEVQLVESGGDLVKPGGSLKLSCAASGFTFSSYGMSWVRQTPDKRLEWVATISSGGSYTYYPDSVKGRFTISRDNAKNILYLQMSSLKSEDTAMYYCARLSTWDWYFDVWGTGTTVTVSSGKPIPNPLLGLDSTHHHHHH

SEQ ID NO: 65 - DNA, 2444 bp, Artificial Sequence
  synthetic construct
atgaagttatgcatattactggccgtcgtggcctttgttggcctctcgctcgggataagatctgcatcggccaccagagcaccaacagcaccgagaccgtggataccctgaccgagaccaacgtgccagtgacccacgccaaggagctgctgcacaccgagcacaacggaatgctgtgcgccaccaatctgggccaccccctgatcctggatacctgcaccatcgagggcctgatctacggcaaccccagctgcgatctgctgctgggaggacgcgagtggtcctacattgtggagcgccccagcgccgtgaacggaacctgctatccaggcaacgtggagaacctggaggagctgcgcaccctgttcagcagctcgagcagctaccagcgcatccagatcttccccgataccatctggaacgtgacctacaccggcaccagcaagagctgcagcgatagcttctaccgcaacatgcgctggctgacccagaagtccggcctgtacccagtgcaggatgcccagtacaccaacaatcgcggcaaggacatcctgttcgtgtggggcatccaccaccccccaaccgataccgcccagaccaatctgtacacccgcaccgataccaccaccagcgtgaccaccgagaatctggatcgcaccttcaagcccgtgatcggcccacgcccactcgtgaatggactgatcggccgcatcaactactattggagcgtgctgaagcccggccagaccctgcgcgtgcgcagcaatggaaatctgatcgccccgtggtacggccacgtgctgagcggagagagccacggccgcattctgaagaccgatctgaacagcggcaactgcgtggtgcagtgccagaccgagaagggcggcctgaatagcaccctgcccttccacaacatctcgaagtacgccttcggaaactgccccaagtacatcggcgtgaagtccctgaagctggccatcggcctgcgcaatgtgccagcccgcagtagtcgcggactgttcggagccattgccggcttcattgagggcggctggccaggactggtggccggatggtacggattccagcacagcaacgatcagggcgtgggaatggccgccgatcgcgatagtacccagaaggccgtggataagatcacctccaaagtgaacaacatcgtggacaagatgaacaagcagtacgagatcatcgaccacgagttcagcgaggtggagacccgcctgaacatgatcaacaacaagatcgacgaccagatccaggatgtgtgggcctacaacgccgagctgctggtgctgctggagaaccagaagaccctggacgagcacgatgccaacgtgaacaatctgtataacaaagtgaagcgcgccctgggcagcaacgccatggaggatggaaagggatgcttcgagctgtaccacaagtgcgacgatcagtgcatggagaccatccgcaacggcacctacaaccgccgcaagtacaaggaggagagccgcctggagcgccagggcagcggctacatcccagaggccccacgcgacggacaggcctatgtgcgcaaggatggcgagtgggtgctgctgagcaccttcctgggttctggctctggtgagatcgtgctgacacagagcccagccttgatggctgctagcccaggcgagaaagtgaccattacctgcagcgtgtccagcagcatcagcagcggcaacttccactggtatcagcagaagtccggcacctcgccgaagctgtggatctacggaacaagcaatctggccagcggagtgccagtgcgctttagtggaagtggaagcggcaccagctacagcctgaccatcagtagcatggaagccgaggatgccgccacctactattgccagcagtggtcgagctaccccttcaccttcggcagtggcaccaagctggaaatcaaaggcggaggcggaagtggtggcggaggttcaggtggtggtggatcaggcggaggtggtagtgaagtgcagctggttgaaagcggcggagacctggttaagccaggcggaagcctgaagctgagttgcgctgccagcggattcaccttcagctcctacggcatgagctgggtccgacagacacccgataagcgcttggagtgggttgccaccattagcagcggaggcagctacacgtactaccccgatagtgtgaagggacgcttcaccatcagccgcgataacgccaagaacatcctgtacctgcagatgagcagcctgaagtccgaggacaccgccatgtattactgcgcccgtctgagcacctgggattggtacttcgatgtgtggggcaccggaaccaccgtgacagttagtagtggttctggctctggtggtaagcctatccctaaccctctcctcggtctcgattctacgcatcatcaccatcaccat

SEQ ID NO: 66 - DNA, 2432 bp, Artificial Sequence
  synthetic construct
atgaagttatgcatattactggccgtcgtggcctttgttggcctctcgctcgggataagatctgcatcggccaccagagcaccaacagcaccgagaccgtggataccctgaccgagaccaacgtgccagtgacccacgccaaggagctgctgcacaccgagcacaacggaatgctgtgcgccaccaatctgggccaccccctgatcctggatacctgcaccatcgagggcctgatctacggcaaccccagctgcgatctgctgctgggaggacgcgagtggtcctacattgtggagcgccccagcgccgtgaacggaacctgctatccaggcaacgtggagaacctggaggagctgcgcaccctgttcagcagctcgagcagctaccagcgcatccagatcttccccgataccatctggaacgtgacctacaccggcaccagcaagagctgcagcgatagcttctaccgcaacatgcgctggctgacccagaagtccggcctgtacccagtgcaggatgcccagtacaccaacaatcgcggcaaggacatcctgttcgtgtggggcatccaccaccccccaaccgataccgcccagaccaatctgtacacccgcaccgataccaccaccagcgtgaccaccgagaatctggatcgcaccttcaagcccgtgatcggcccacgcccactcgtgaatggactgatcggccgcatcaactactattggagcgtgctgaagcccggccagaccctgcgcgtgcgcagcaatggaaatctgatcgccccgtggtacggccacgtgctgagcggagagagccacggccgcattctgaagaccgatctgaacagcggcaactgcgtggtgcagtgccagaccgagaagggcggcctgaatagcaccctgcccttccacaacatctcgaagtacgccttcggaaactgccccaagtacatcggcgtgaagtccctgaagctggccatcggcctgcgcaatgtgccagcccgcagtagtcgcggactgttcggagccattgccggcttcattgagggcggctggccaggactggtggccggatggtacggattccagcacagcaacgatcagggcgtgggaatggccgccgatcgcgatagtacccagaaggccgtggataagatcacctccaaagtgaacaacatcgtggacaagatgaacaagcagtacgagatcatcgaccacgagttcagcgaggtggagacccgcctgaacatgatcaacaacaagatcgacgaccagatccaggatgtgtgggcctacaacgccgagctgctggtgctgctggagaaccagaagaccctggacgagcacgatgccaacgtgaacaatctgtataacaaagtgaagcgcgccctgggcagcaacgccatggaggatggaaagggatgcttcgagctgtaccacaagtgcgacgatcagtgcatggagaccatccgcaacggcacctacaaccgccgcaagtacaaggaggagagccgcctggagcgccagggcagcggctacatcccagaggccccacgcgacggacaggcctatgtgcgcaaggatggcgagtgggtgctgctgagcaccttcctgggttctggctctggtgatatcgtgatgacccagtcgccaagcagtctggctgtgtccgtgggacagaaagtgaccatgagctgcaccagcagccaggtgctgctgcacagccccaaccagaagaattacctggcctggtatcagcagaagcccggccaaagtccgaagctgctggtctactttgccagcacacgcgagagcggagtgccagatcgttttaccggaagcggcagcggcaccgatttcaccctgacaattagtagcgtgcaggccgaggatctggccgtgtattactgccagcagcactacagcaccccgctgacatttggcgccggaacgaagctggaactgaaaggcggaggtggtagtggtggcggaggatcaggtggtggtggttctggcggtggtggaagtgaagtgcaactgcagcagagcggcccagagctggtcaaaccaggtgccagcgtgaagatcagctgcaaggccagcggatacaccttcaccgattactacatcaactgggtcaagcagagccacggcaagagcctggaatggatcggcgatatcaaccccaccaacggcgatagcacctacagccagaagttcaagggcaaagccacgctgaccgtggataagagtagcagcaccgcctacatggaactgcgcagcctgacaagcgaagtgtccgccgtgtactattgcgcccgtgattacgccatggattactggggacagggcaccagtgtgaccgttagtagtggtaagcctatccctaaccctctcctcggtctcgattctacgcatcatcaccatcaccat

SEQ ID NO: 67 - DNA, 2426 bp, Artificial Sequence
  synthetic construct
atgaagttatgcatattactggccgtcgtggcctttgttggcctctcgctcgggataagatctgcatcggccaccagagcaccaacagcaccgagaccgtggataccctgaccgagaccaacgtgccagtgacccacgccaaggagctgctgcacaccgagcacaacggaatgctgtgcgccaccaatctgggccaccccctgatcctggatacctgcaccatcgagggcctgatctacggcaaccccagctgcgatctgctgctgggaggacgcgagtggtcctacattgtggagcgccccagcgccgtgaacggaacctgctatccaggcaacgtggagaacctggaggagctgcgcaccctgttcagcagctcgagcagctaccagcgcatccagatcttccccgataccatctggaacgtgacctacaccggcaccagcaagagctgcagcgatagcttctaccgcaacatgcgctggctgacccagaagtccggcctgtacccagtgcaggatgcccagtacaccaacaatcgcggcaaggacatcctgttcgtgtggggcatccaccaccccccaaccgataccgcccagaccaatctgtacacccgcaccgataccaccaccagcgtgaccaccgagaatctggatcgcaccttcaagcccgtgatcggcccacgcccactcgtgaatggactgatcggccgcatcaactactattggagcgtgctgaagcccggccagaccctgcgcgtgcgcagcaatggaaatctgatcgccccgtggtacggccacgtgctgagcggagagagccacggccgcattctgaagaccgatctgaacagcggcaactgcgtggtgcagtgccagaccgagaagggcggcctgaatagcaccctgcccttccacaacatctcgaagtacgccttcggaaactgccccaagtacatcggcgtgaagtccctgaagctggccatcggcctgcgcaatgtgccagcccgcagtagtcgcggactgttcggagccattgccggcttcattgagggcggctggccaggactggtggccggatggtacggattccagcacagcaacgatcagggcgtgggaatggccgccgatcgcgatagtacccagaaggccgtggataagatcacctccaaagtgaacaacatcgtggacaagatgaacaagcagtacgagatcatcgaccacgagttcagcgaggtggagacccgcctgaacatgatcaacaacaagatcgacgaccagatccaggatgtgtgggcctacaacgccgagctgctggtgctgctggagaaccagaagaccctggacgagcacgatgccaacgtgaacaatctgtataacaaagtgaagcgcgccctgggcagcaacgccatggaggatggaaagggatgcttcgagctgtaccacaagtgcgacgatcagtgcatggagaccatccgcaacggcacctacaaccgccgcaagtacaaggaggagagccgcctggagcgccagggcagcggctacatcccagaggccccacgcgacggacaggcctatgtgcgcaaggatggcgagtgggtgctgctgagcaccttcctgggttctggctctggtgaagtgcaactgcaacaaagcggcccagagctggttaagccaggtgccagtgtgaagatgagctgcaaggccagcggctacaccttcaccaactacgtgctgcactgggtcaagcagaagcccggccaaggcctggaatggatcggctacatcaacccctacaacgatggcaccaagttcaacgagaagttcaagggcaaagccacgctgaccagcgataccagtagcagcaccgccttcatggaactgagcagcctgacctccgaagatagcgccgtgtactattgcgcccgtggcgataatctgcgcccctactacttcgattactggggccagggaacgaccctgacagttagttcaggtggcggaggtagcggaggtggtggatcaggtggtggtggaagtggtggcggtggatcccagattgtgctgacacacagccccgccatcatgagtgctagcccaggcgagaaagtgaccatgacatgcagtgccagcagcagcgtgtccttcatgtattggtatcagcaaaagccgggcagcagcccgcgtctgctgctgtatgatacaagctccctgagcagcggagtgcccgtgcgttttagtggaagcggatccggaaccagctactccctgaccatcagtcgcatggaagccgaagatgccgccacgtactactgccagcagtggtcccgttatccgccaacattcggcggaggcaccaagctggaaatcaagggtaagcctatccctaaccctctcctcggtctcgattctacgcatcatcaccatcaccat

SEQ ID NO: 68 - protein, 522 aa, Artificial Sequence
  synthetic polypeptide
  MISC_FEATURE: CD33 SIGNAL-IG10 scFv-(Glycine4Serine)4 linker-CD83 scFv-Ctag
MPLLLLLPLLWAGALAMDILMTQSPSSMSVSLGDTVIITCHASQGISSNIGWLQQKPGKSFKGLIYHATNLEDGVPSRFSGGGSGADYSLTISSLESEDFADYYCVQYGQFPFTFGSGTKLEIKGGGGSGGGGSGGGGSEVQLQQSVAELVRPGASVKLSCTASGFNIKNTYMHWVKQRPEQGLEWIGRIDPANGNTRYAPKFQGKATITADTSSNTAYLQLSSLTSEDTAIYYCARYYFGPDYWGQGTTLTVSSGGGGSGGGGSGGGGSGGGGSDIVMTQSPSSLAVSVGQKVTMSCTSSQVLLHSPNQKNYLAWYQQKPGQSPKLLVYFASTRESGVPDRFTGSGSGTDFTLTISSVQAEDLAVYYCQQHYSTPLTFGAGTKLELKGGGGSGGGGSGGGGSEVQLQQSGPELVKPGASVKISCKASGYTFTDYYINWVKQSHGKSLEWIGDINPTNGDSTYSQKFKGKATLTVDKSSSTAYMELRSLTSEVSAVYYCARDYAMDYWGQGTSVTVSSEPEA

SEQ ID NO: 69 - DNA, 1569 bp, Artificial Sequence
  synthetic construct
atgcccttgttgctgctgctgccactgctgtgggccggagccctggccatggatgatattctgatgactcaatcgccaagctccatgagtgtctcgctcggcgacacggtaataataacttgtcacgctagtcagggcattagcagtaacataggatggctccagcaaaaacccggcaagagctttaagggtttgatttaccacgcaacgaacctggaagatggagtgccgagccgatttagtggcggaggcagcggtgccgattacagcttgacaatatcgagtctggaatcggaggactttgccgattattattgtgtacagtacggtcagtttccatttacgttcggaagcggtacgaagttggagataaaaggcggaggcggaagtggcggcggaggaagcggaggcggaggatccgaggttcaactgcaacaatcggttgcagaattggtacggccaggagcaagtgtgaagctgagctgcacggcgagcggtttcaacataaagaacacttacatgcactgggtgaagcaacggccagagcagggcttggagtggattggacgcattgatcccgccaacggcaacactaggtacgcgccgaaattccaaggtaaagctacgatcactgcagatacatcgagtaacaccgcctacctccaactctcgagtctcactagcgaggatacggccatctattactgcgcacggtattatttcggacctgattactggggtcagggaacgactctgactgtatccagtggcggaggcggaagtggcggcggaggaagcggaggcggaggatccggcggaggcggcagtgatatcgtgatgacccagtcgccaagcagtctggctgtgtccgtgggacagaaagtgaccatgagctgcaccagcagccaggtgctgctgcacagccccaaccagaagaattacctggcctggtatcagcagaagcccggccaaagtccgaagctgctggtctactttgccagcacacgcgagagcggagtgccagatcgttttaccggaagcggcagcggcaccgatttcaccctgacaattagtagcgtgcaggccgaggatctggccgtgtattactgccagcagcactacagcaccccgctgacatttggcgccggaacgaagctggaactgaaaggcggaggtggtagtggtggcggaggatcaggtggtggtggttctgaagtgcaactgcagcagagcggcccagagctggtcaaaccaggtgccagcgtgaagatcagctgcaaggccagcggatacaccttcaccgattactacatcaactgggtcaagcagagccacggcaagagcctggaatggatcggcgatatcaaccccaccaacggcgatagcacctacagccagaagttcaagggcaaagccacgctgaccgtggataagagtagcagcaccgcctacatggaactgcgcagcctgacaagcgaagtgtccgccgtgtactattgcgcccgtgattacgccatggattactggggacagggcaccagtgtgaccgttagtagtgagccagaggct

SEQ ID NO: 70 - protein, 4 aa, Artificial Sequence
  synthetic peptide
GFLG

SEQ ID NO: 71 - protein, 4 aa, Artificial Sequence
  synthetic peptide
ALAL

SEQ ID NO: 72 - DNA, 2574 bp, Artificial Sequence
  synthetic construct
atgaagctgtgcatcctgctggccgtggtggcctttgtgggattgagtttgggacgtagtccatggcccggggatcagatctgtattggctatcacgccaataacagcaccgaacaagttgacaccattatggaaaagaacgtaacagtcacgcatgcccaggacattctcgagaaaacccacaatggcaaactctgtgacttgaacggcgtcaaacctctcatactgaaagactgttcggtcgcgggctggctcctcggtaatcctatgtgcgacgaatttatccgcgtacctgagtggtcgtatattgttgagcgggcaaacccggcaaatgatctctgctatcctggaagcctcaacgactatgaagaactcaaacacctcctctcgcgcataaatcacttcgaaaaaatactcatcataccaaagagcagttggcctaaccacgagacatcgctcggcgtttcggcagcctgtccctatcagggaactccatcgtttttccgtaatgtagtatggctcatcaaaaagaatgacgcctatcctacaattaaaattagctataataacaccaatcgcgaagacctcctgattatgtggggtattcatcactccaataacgccgaagaacagaccaacttgtacaaaaaccccactacctatataagtgtgggtaccagtacactgaaccaaaggctggttccaaagatagctactcgtagccaggttaatggtcaacggggacgtatggacttcttttggacaattttgaaaccaaacgacgcaatacattttgaaagcaatggtaactttatcgccccagagtacgcctataagatcgtcaagaagggagactcgacgataatgaagagtgaagtagaatacggccactgcaatacgaaatgtcaaacgccagtcggtgccatcaacagctcgatgccgttccataacatccatccgctgacgattggagagtgcccgaaatacgtaaagagcaataaattggtcttggccactggcctccgtaatagtccccagggcgagacacggggtctgttcggtgccatcgctggcttcattgagggaggttggcagggtatggtggacggatggtacggctatcatcatagtaatgaacagggtagtggttacgcagcggataaggagtccacccagaaggctatcgatggagtgacaaacaaggtgaacagtattattgataaaatgaacacccaatttgaagcagtgggacgagagttcaataatctcgaacggcgcatcgaaaacctgaataaaaagatggaagacggattcctcgacgtctggacctacaacgctgaactgttggtgctcatggaaaacgagcggacgctcgatttccacgatagcaatgttaagaatctgtatgacaaagtccggctccaattgcgagacaacgccaaagaactgggaaacggttgttttgagttctatcataaatgtgataacgagtgcatggaaagtgtccgaaatggaacatacgattacccacagtattcggaggaagctcgtttgaaaagggaagaaatatccggcgtaaaactcgaaagtatcggaacctatcaagcggccgcaggcagcggctacatcccagaggccccacgcgacggacaggcctatgtgcgcaaggatggcgagtgggtgctgctgagcaccttcctgttaattaagaattcgcggccgctcgagggaagtggaagcggagatatcgtgatgacccagtcgccaagcagtctggctgtgtccgtgggacagaaagtgaccatgagctgcaccagcagccaggtgctgctgcacagccccaaccagaagaattacctggcctggtatcagcagaagcccggccaaagtccgaagctgctggtctactttgccagcacacgcgagagcggagtgccagatcgttttaccggaagcggcagcggcaccgatttcaccctgacaattagtagcgtgcaggccgaggatctggccgtgtattactgccagcagcactacagcaccccgctgacatttggcgccggaacgaagctggaactgaaaggcggaggtggtagtggtggcggaggatcaggtggtggtggttctggcggtggtggaagtgaagtgcaactgcagcagagcggcccagagctggtcaaaccaggtgccagcgtgaagatcagctgcaaggccagcggatacaccttcaccgattactacatcaactgggtcaagcagagccacggcaagagcctggaatggatcggcgatatcaaccccaccaacggcgatagcacctacagccagaagttcaagggcaaagccacgctgaccgtggataagagtagcagcaccgcctacatggaactgcgcagcctgacaagcgaagtgtccgccgtgtactattgcgcccgtgattacgccatggattactggggacagggcaccagtgtgaccgttagtagttctagagggcccttcgaaggtaagcctatccctaaccctctcctcggtctcgattctacgcgtgaaggcggcgagccagaggcttaa

SEQ ID NO: 73 - DNA, 1788 bp, Artificial Sequence
  synthetic construct
  misc_feature: BIP-H5HA-Foldon
atgaagctgtgcatcctgctggccgtggtggcctttgtgggattgagtttgggacgtagtccatggcccggggatcagatctgtattggctatcacgccaataacagcaccgaacaagttgacaccattatggaaaagaacgtaacagtcacgcatgcccaggacattctcgagaaaacccacaatggcaaactctgtgacttgaacggcgtcaaacctctcatactgaaagactgttcggtcgcgggctggctcctcggtaatcctatgtgcgacgaatttatccgcgtacctgagtggtcgtatattgttgagcgggcaaacccggcaaatgatctctgctatcctggaagcctcaacgactatgaagaactcaaacacctcctctcgcgcataaatcacttcgaaaaaatactcatcataccaaagagcagttggcctaaccacgagacatcgctcggcgtttcggcagcctgtccctatcagggaactccatcgtttttccgtaatgtagtatggctcatcaaaaagaatgacgcctatcctacaattaaaattagctataataacaccaatcgcgaagacctcctgattatgtggggtattcatcactccaataacgccgaagaacagaccaacttgtacaaaaaccccactacctatataagtgtgggtaccagtacactgaaccaaaggctggttccaaagatagctactcgtagccaggttaatggtcaacggggacgtatggacttcttttggacaattttgaaaccaaacgacgcaatacattttgaaagcaatggtaactttatcgccccagagtacgcctataagatcgtcaagaagggagactcgacgataatgaagagtgaagtagaatacggccactgcaatacgaaatgtcaaacgccagtcggtgccatcaacagctcgatgccgttccataacatccatccgctgacgattggagagtgcccgaaatacgtaaagagcaataaattggtcttggccactggcctccgtaatagtccccagggcgagacacggggtctgttcggtgccatcgctggcttcattgagggaggttggcagggtatggtggacggatggtacggctatcatcatagtaatgaacagggtagtggttacgcagcggataaggagtccacccagaaggctatcgatggagtgacaaacaaggtgaacagtattattgataaaatgaacacccaatttgaagcagtgggacgagagttcaataatctcgaacggcgcatcgaaaacctgaataaaaagatggaagacggattcctcgacgtctggacctacaacgctgaactgttggtgctcatggaaaacgagcggacgctcgatttccacgatagcaatgttaagaatctgtatgacaaagtccggctccaattgcgagacaacgccaaagaactgggaaacggttgttttgagttctatcataaatgtgataacgagtgcatggaaagtgtccgaaatggaacatacgattacccacagtattcggaggaagctcgtttgaaaagggaagaaatatccggcgtaaaactcgaaagtatcggaacctatcaagcggccgcaggcagcggctacatcccagaggccccacgcgacggacaggcctatgtgcgcaaggatggcgagtgggtgctgctgagcaccttcctgtctagagggcccttcgaaggtaagcctatccctaaccctctcctcggtctcgattctacgcgtgaaggcggcgagccagaggcttaa

SEQ ID NO: 74 - DNA, 1632 bp, Artificial Sequence
  synthetic construct
  misc_feature: H9HA-Foldon
atggaggctattagtctgatgattattctgctggtggtgacaacctcaaatgccgataaaatttgtattggacatcagtctactaacagcacagagactgtggacaccctgacagaatccaacatccccgtgacccaggcaaaagagctgctgcacacagaacacaacgggatgctgtgcgccaccaatctgggcaggcccctgatcctggacacttgcaccgtggagggcctgatctacggaaaccctagctgcgatctgctgctgggcggaagagagtggagctacatcgtggaaaggccctccgcagtgaatggaacatgctaccctgggaacgtggaaaatctggaggaactgcggatgctgttcagctccgccagctcctaccagagaatccagatttttcctgatgctatctggaacgtgacctacgacggaacaagcaaatcctgcagcaactccttctacagaaatatgaggtggctgacccagaagaacgggaattacccaattcaggatgctcagtacacaaacaatcggggaaaggacatcctgtttatttgggggatccaccacccccctactgataccacacagaccaacctgtacacaagaactgacactaccacaagcgtgactaccgagaatctggatagaactttcaaacctctgatcggcccaaggcccctggtgaacggcctgattggacgcatcaattactactggagcgtgctgaagcctggacagactctgcgcgtgcggtccaacggcaatctgatcgccccatggtacggacacgtgctgagcggagagtcccacggaaggattctgaagaccgacctgaaatccgggaactgcgtggtgcagtgccagactgaaaaagggggcctgaacagcaccctgccattccacaatatctccaaatacgccttcggcacatgccccaagtacatcggagtgaagagcctgaaactggctattggcctgcgcaacgtgccagcaaagtccaatcgggggctgttcggcgcaattgccgggtttatcgagggaggatggccaggcctggtggctggatggtacgggttccagcacagcaacgatcagggagtgggaatggcagctgacaggggaagcacacagaaggcagtggataaaatcacttccaaggtgaacaacatcatcgataagatgaacaggcagtacgagatcattgaccacgaattttccgagattgaaacacgcctgaatatgattaacaacaagatcgatgaccagattcaggacgtgtgggcttacaacgcagagctgctggtgctgctggaaaatcagaagactctggatgagcacgacgccaacgtgaacaatctgtacaataaggtgaaaagagccctggggagcaacgctatggaggatgggaaaggctgctttgaactgtaccacaagtgcgatgaccagtgcatggaaacaatcaggaacggcacttacaatagaaggaagtacacagaggaaagccgcctggagaggcagggatccggatacattcctgaagcaccacgcgatggacaggcctacgtgaggaaggatggggagtgggtgctgctgagtacatttctgggggaacctgaggcataa

SEQ ID NO: 75 - DNA, 2382 bp, Artificial Sequence
  synthetic construct
  misc_feature: H9HA-Foldon-CD83scFv
atggaggctattagtctgatgattattctgctggtggtgacaacctcaaatgccgataaaatttgtattggacatcagtctactaacagcacagagactgtggacaccctgacagaatccaacatccccgtgacccaggcaaaagagctgctgcacacagaacacaacgggatgctgtgcgccaccaatctgggcaggcccctgatcctggacacttgcaccgtggagggcctgatctacggaaaccctagctgcgatctgctgctgggcggaagagagtggagctacatcgtggaaaggccctccgcagtgaatggaacatgctaccctgggaacgtggaaaatctggaggaactgcggatgctgttcagctccgccagctcctaccagagaatccagatttttcctgatgctatctggaacgtgacctacgacggaacaagcaaatcctgcagcaactccttctacagaaatatgaggtggctgacccagaagaacgggaattacccaattcaggatgctcagtacacaaacaatcggggaaaggacatcctgtttatttgggggatccaccacccccctactgataccacacagaccaacctgtacacaagaactgacactaccacaagcgtgactaccgagaatctggatagaactttcaaacctctgatcggcccaaggcccctggtgaacggcctgattggacgcatcaattactactggagcgtgctgaagcctggacagactctgcgcgtgcggtccaacggcaatctgatcgccccatggtacggacacgtgctgagcggagagtcccacggaaggattctgaagaccgacctgaaatccgggaactgcgtggtgcagtgccagactgaaaaagggggcctgaacagcaccctgccattccacaatatctccaaatacgccttcggcacatgccccaagtacatcggagtgaagagcctgaaactggctattggcctgcgcaacgtgccagcaaagtccaatcgggggctgttcggcgcaattgccgggtttatcgagggaggatggccaggcctggtggctggatggtacgggttccagcacagcaacgatcagggagtgggaatggcagctgacaggggaagcacacagaaggcagtggataaaatcacttccaaggtgaacaacatcatcgataagatgaacaggcagtacgagatcattgaccacgaattttccgagattgaaacacgcctgaatatgattaacaacaagatcgatgaccagattcaggacgtgtgggcttacaacgcagagctgctggtgctgctggaaaatcagaagactctggatgagcacgacgccaacgtgaacaatctgtacaataaggtgaaaagagccctggggagcaacgctatggaggatgggaaaggctgctttgaactgtaccacaagtgcgatgaccagtgcatggaaacaatcaggaacggcacttacaatagaaggaagtacacagaggaaagccgcctggagaggcagggatccggatacattcctgaagcaccacgcgatggacaggcctacgtgaggaaggatggggagtgggtgctgctgagtacatttctggggcccggggatattgtgatgacccagtctcctagtagcctggccgtgtccgtggggcagaaagtgaccatgtcttgtacctcctctcaggtgctgctgcactcccctaaccagaaaaattacctggcctggtaccagcagaaacctggccagagccctaagctgctggtgtacttcgccagcactagagagtccggcgtgccagataggttcacaggatccgggagcggcactgactttaccctgacaatcagctccgtgcaggccgaggatctggccgtgtactactgccagcagcactacagcacccccctgacatttggagcagggacaaaactggaactgaagggcggaggaggatccggaggaggaggaagcggaggaggagggtccggcggaggaggaagcgaggtgcagctgcagcagagcggaccagaactggtgaaacccggagcaagcgtgaaaatctcctgcaaggccagcggatacactttcaccgattactacattaactgggtgaaacagtcccacgggaagagcctggaatggatcggcgatattaaccctactaatggagactccacctacagccagaagtttaaaggcaaggctacactgactgtggacaagagctccagcaccgcatacatggagctgagatccctgacaagcgaagtgtccgccgtgtactactgtgccagagattatgctatggactattggggccaggggacaagcgtgactgtgagttccgaacctgaggcataa

SEQ ID NO: 76 - protein, 857 aa, Artificial Sequence
  synthetic polypeptide
MKLCILLAVVAFVGLSLGRSPWPGDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHLLSRINHFEKILIIPKSSWPNHETSLGVSAACPYQGTPSFFRNVVWLIKKNDAYPTIKISYNNTNREDLLIMWGIHHSNNAEEQTNLYKNPTTYISVGTSTLNQRLVPKIATRSQVNGQRGRMDFFWTILKPNDAIHFESNGNFIAPEYAYKIVKKGDSTIMKSEVEYGHCNTKCQTPVGAINSSMPFHNIHPLTIGECPKYVKSNKLVLATGLRNSPQGETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQAAAGSGYIPEAPRDGQAYVRKDGEWVLLSTFLLIKNSRPLEGSGSGDIVMTQSPSSLAVSVGQKVTMSCTSSQVLLHSPNQKNYLAWYQQKPGQSPKLLVYFASTRESGVPDRFTGSGSGTDFTLTISSVQAEDLAVYYCQQHYSTPLTFGAGTKLELKGGGGSGGGGSGGGGSGGGGSEVQLQQSGPELVKPGASVKISCKASGYTFTDYYINWVKQSHGKSLEWIGDINPTNGDSTYSQKFKGKATLTVDKSSSTAYMELRSLTSEVSAVYYCARDYAMDYWGQGTSVTVSSSRGPFEGKPIPNPLLGLDSTREGGEPEA

SEQ ID NO: 77 - protein, 581 aa, Artificial Sequence
  synthetic polypeptide
MKLCILLAVVAFVGLSLGRSPWPGDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHLLSRINHFEKILIIPKSSWPNHETSLGVSAACPYQGTPSFFRNVVWLIKKNDAYPTIKISYNNTNREDLLIMWGIHHSNNAEEQTNLYKNPTTYISVGTSTLNQRLVPKIATRSQVNGQRGRMDFFWTILKPNDAIHFESNGNFIAPEYAYKIVKKGDSTIMKSEVEYGHCNTKCQTPVGAINSSMPFHNIHPLTIGECPKYVKSNKLVLATGLRNSPQGETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQAAAGSGYIPEAPRDGQAYVRKDGEWVLLSTFLLIKIKTREGGEPEA

SEQ ID NO: 78 - protein, 793 aa, Artificial Sequence
  synthetic polypeptide
MEAISLMIILLVVTTSNADKICIGHQSTNSTETVDTLTESNIPVTQAKELLHTEHNGMLCATNLGRPLILDTCTVEGLIYGNPSCDLLLGGREWSYIVERPSAVNGTCYPGNVENLEELRMLFSSASSYQRIQIFPDAIWNVTYDGTSKSCSNSFYRNMRWLTQKNGNYPIQDAQYTNNRGKDILFIWGIHHPPTDTTQTNLYTRTDTTTSVTTENLDRTFKPLIGPRPLVNGLIGRINYYWSVLKPGQTLRVRSNGNLIAPWYGHVLSGESHGRILKTDLKSGNCVVQCQTEKGGLNSTLPFHNISKYAFGTCPKYIGVKSLKLAIGLRNVPAKSNRGLFGAIAGFIEGGWPGLVAGWYGFQHSNDQGVGMAADRGSTQKAVDKITSKVNNIIDKMNRQYEIIDHEFSEIETRLNMINNKIDDQIQDVWAYNAELLVLLENQKTLDEHDANVNNLYNKVKRALGSNAMEDGKGCFELYHKCDDQCMETIRNGTYNRRKYTEESRLERQGSGYIPEAPRDGQAYVRKDGEWVLLSTFLGPGDIVMTQSPSSLAVSVGQKVTMSCTSSQVLLHSPNQKNYLAWYQQKPGQSPKLLVYFASTRESGVPDRFTGSGSGTDFTLTISSVQAEDLAVYYCQQHYSTPLTFGAGTKLELKGGGGSGGGGSGGGGSGGGGSEVQLQQSGPELVKPGASVKISCKASGYTFTDYYINWVKQSHGKSLEWIGDINPTNGDSTYSQKFKGKATLTVDKSSSTAYMELRSLTSEVSAVYYCARDYAMDYWGQGTSVTVSSEPEA

SEQ ID NO: 79 - protein, 543 aa, Artificial Sequence
  synthetic polypeptide
MEAISLMIILLVVTTSNADKICIGHQSTNSTETVDTLTESNIPVTQAKELLHTEHNGMLCATNLGRPLILDTCTVEGLIYGNPSCDLLLGGREWSYIVERPSAVNGTCYPGNVENLEELRMLFSSASSYQRIQIFPDAIWNVTYDGTSKSCSNSFYRNMRWLTQKNGNYPIQDAQYTNNRGKDILFIWGIHHPPTDTTQTNLYTRTDTTTSVTTENLDRTFKPLIGPRPLVNGLIGRINYYWSVLKPGQTLRVRSNGNLIAPWYGHVLSGESHGRILKTDLKSGNCVVQCQTEKGGLNSTLPFHNISKYAFGTCPKYIGVKSLKLAIGLRNVPAKSNRGLFGAIAGFIEGGWPGLVAGWYGFQHSNDQGVGMAADRGSTQKAVDKITSKVNNIIDKMNRQYEIIDHEFSEIETRLNMINNKIDDQIQDVWAYNAELLVLLENQKTLDEHDANVNNLYNKVKRALGSNAMEDGKGCFELYHKCDDQCMETIRNGTYNRRKYTEESRLERQGSGYIPEAPRDGQAYVRKDGEWVLLSTFLGEPEA

SEQ ID NO: 80 - protein, 511 aa, Artificial Sequence
  synthetic polypeptide
  MISC_FEATURE: H5N8 ectodomain
DQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHLLSRINHFEKILIIPKSSWPNHETSLGVSAACPYQGTPSFFRNVVWLIKKNDAYPTIKISYNNTNREDLLIMWGIHHSNNAEEQTNLYKNPTTYISVGTSTLNQRLVPKIATRSQVNGQRGRMDFFWTILKPNDAIHFESNGNFIAPEYAYKIVKKGDSTIMKSEVEYGHCNTKCQTPVGAINSSMPFHNIHPLTIGECPKYVKSNKLVLATGLRNSPQGETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQ

(19) United States
(12) Patent Application Publication
(10) Pub. No.: US 2025/0262294 A1
(43) Pub. Date: Aug. 21, 2025

(54) VACCINE

(71) Applicant: THE PIRBRIGHT INSTITUTE, Pirbright , Woking (GB)

(72) Inventors: Munir Iqbal, Pirbright, Woking (GB); Angita Shrestha, Pirbright, Woking (GB); Jean-Remy Sadeyen, Pirbright, Woking (GB)

(21) Appl. No.: 18/032,367

(22) PCT Filed: Oct. 25, 2021
(86) PCT No.: PCT/GB2021/052769

§ 371 Date: Apr. 18, 2023

(30) Foreign Application Priority Data
Oct. 26, 2020 (GB) 2016954.6

(51) Int. Cl. A61K 39/145; A61K 39/00; A61K 39/385; A61P 31/16; C07K 14/005; C07K 16/28; C12N 7/00; C12N 15/85

(52) U.S. Cl. CPC A61K 39/145; A61K 39/385; A61P 31/16; C07K 14/005; C07K 16/2803; C12N 7/00; C12N 15/85; A61K 2039/552; A61K 2039/575; A61K 2039/6056; C07K 2317/622; C12N 2760/16122; C12N 2760/16134

(57) ABSTRACT

The present invention relates to a genetically engineered protein comprising: at least one binding domain which is capable of binding to a cell surface protein on an avian antigen presenting cell; and a) at least one antigenic polypeptide orb) at least one binding domain which is capable of binding to at least one antigenic polypeptide. The present invention also relates to avian vaccines comprising at least one binding domain which is capable of binding to a cell surface protein on an avian antigen presenting cell; and a) at least one antigenic polypeptide or b) at least one binding domain which is capable of binding to at least one antigenic polypeptide and to the use of such vaccines to treat and/or prevent disease in avian subjects.

### FIELD OF THE INVENTION

The present invention relates to engineered proteins for targeted antigen delivery to avian antigen presenting cells. The present invention also relates to avian vaccines which are targeted antigen delivery vaccines (TADV) or antigen targeted vaccines (ATV). The vaccines protect avian subjects against subsequent challenge with pathogen which comprises the at least one antigen. The present invention also relates to the use of such vaccines to treat and/or prevent disease in avian subjects.

### BACKGROUND TO THE INVENTION

Vaccines are major tools to reduce the impact of disease, such as cancer or infectious disease, in farmed animals and humans.

Several approaches are employed in poultry vaccine design. Currently, three main formulations of vaccines for poultry diseases are practised. These include inactivated vaccines, live-attenuated vaccines and vectored vaccines.

The majority of licensed poultry vaccines are inactivated vaccines and largely produced in embryonated hen's eggs and after inactivation, these viruses are reconstituted with adjuvants. These vaccines have some inherent drawbacks including sub-optimal protection to vaccinated birds. For example, despite administration of multiple doses to individual birds (e.g. two to five doses to layers during one year), the infectious virus may continue to circulate in vaccinated flocks; vaccinated birds are not easy to distinguish from naturally infected birds by common serological tests; residual pathogenicity from inactivated vaccines can result in pathogens that can contribute to or exacerbate the outbreak; biohazards associated with manufacturing the vaccines; and low vaccine yield from embryonated hens' eggs when high pathogenicity strains are used as vaccine seed virus, has hindered their cost-effective production.

Many vaccines administered in multiple doses, inducing sub-optimal immunity that might protect from clinical disease and death, do not prevent shedding of infectious pathogens from vaccinated animals. Thus, the endemic cycle of disease continues.

In recent years, various strategies have been developed to enhance the immunogenicity of vaccines. One such strategy is ATV or recombinant TADV technology. This vaccine platform selectively delivers antigen to host immune cells known as antigen-presenting cells (APCs) that capture, process and present antigens for initiation and maintenance of immune responses whereby protective antigens are selectively delivered to professional APCs such as dendritic cells (DC), macrophages and B cells. These cells capture, process and present antigens to T lymphocytes for initiation and maintenance of immune responses. Previously, various studies explored antibody-based targeting to mammalian APCs via DC receptor for Endocytosis-205 (Dec205) with limited success. Dec205 is a C-type lectin endocytic receptor of the mannose receptor family and is shown to enhance antigen presentation via the Major Histocompatibility Complex II (MHCII) pathway. There are limited data available for targeting avian APCs for modulating immunogenicity of poultry vaccines. There is a need for improved vaccines to control disease and/or prevent the spread of disease in avian subjects.

### SUMMARY OF THE INVENTION

Generally, the invention relates to directing delivery of antigens to avian immune cells and enhancing potency and efficacy of avian vaccines.

In one aspect, the present invention provides an engineered protein comprising: at least one binding domain which is capable of binding to a cell surface protein on an avian antigen presenting cell; and at least one antigenic polypeptide.

In one aspect, the engineered protein is a genetically engineered protein.

Suitably, the antigenic polypeptide may comprise part or all of an antigen.

Suitably, the antigenic polypeptide may be part or all of an antigen (for example, the antigenic polypeptide may comprise or consist of one or more domains of the antigen).

In one aspect, the present invention provides an engineered protein (such as a genetically engineered protein) comprising: at least one binding domain which is capable of binding to a cell surface protein on an avian antigen presenting cell; and at least one binding domain which is capable of binding to at least one antigenic polypeptide.

The at least one binding domain and at least one antigenic polypeptide or the at least two binding domains may be comprised in a single recombinant protein.

The at least one binding domain capable of binding to a cell surface protein may be operably linked to the at least one antigenic polypeptide or the at least one binding domain which is capable of binding to an antigen.

The antigen presenting cell may be at least one of a dendritic cell, macrophage, B cell, T cell or natural killer cell.

The avian antigen presenting cell may be selected from at least one of a dendritic cell, macrophage, B cell, T cell or natural killer cell.

The cell surface protein may be selected from an immunoglobulin family protein, an integrin family receptor or a C-type lectin.

The cell surface protein may be selected from CD83, CD11c or Dec205. The cell surface protein may be CD83.

The at least one antigenic polypeptide may be an avian influenza virus antigenic polypeptide, such as hemagglutinin antigenic polypeptide.

The engineered protein (such as a genetically engineered protein) according to the present invention may comprise a signal peptide.

The engineered protein (such as a genetically engineered protein) according to the present invention may comprise a domain which improves solubilisation, stabilization and/or folding of the engineered protein. In particular, the domain may improve solubilisation, stabilization and/or folding of the antigen.

The binding domain may be based on or may be the antigen binding site of an antibody or an antibody fragment such as a single-chain variable fragment (scFv), Fv, F(ab′) or F(ab′)2.

In another aspect, the present invention provides a nucleic acid construct which comprises a first polynucleotide which encodes at least one binding domain which is capable of binding to a cell surface protein on an avian antigen presenting cell; and a second polynucleotide which encodes at least one antigenic polypeptide or at least one binding domain which is capable of binding to at least one antigenic polypeptide.

In a further aspect, the present invention provides a vector which comprises a nucleic acid construct according to the present invention.

In another aspect, the present invention provides an engineered cell expressing an engineered protein according to the present invention, or comprising a nucleic acid construct according to the present invention, or comprising a vector according to the present invention.

In another aspect, the present invention provides an avian vaccine comprising an engineered protein (such as a genetically engineered protein) according to the present invention, a nucleic acid construct according to the present invention or a vector according to the present invention and a pharmaceutically acceptable carrier.

In one aspect, there is provided an avian vaccine according to the present invention for use in treating and/or preventing disease in a subject.

In another aspect, the present invention provides a method for treating and/or preventing a disease in a subject which comprises the step of administering to a subject an effective amount of a vaccine according to the present invention.

Suitably, administration of a vaccine according to the present invention may elicit a humoral and/or cellular immune response in the subject.

Suitably, administration of a vaccine according to the present invention may decrease the challenge pathogen load (such as viral load, bacterial load or parasitic load) in the subject.

Suitably, administration of a vaccine according to the present invention may elicit production of cross-reactive antibodies in the subject.

Suitably, the subject may be an avian subject.

Suitably the subject may be poultry, for example the subject may be selected from a chicken, turkey, duck, quail, pigeon or goose.

In another aspect, the present invention provides a method for the preparation of the vaccine according to the present invention, the method comprising the step of admixing a genetically engineered protein according to the present invention, a nucleic acid construct according to the present invention, and/or a vector according to the present invention, and a pharmaceutically acceptable carrier.

In a further aspect, the present invention provides the use of an engineered protein (such as a genetically engineered protein) according to the present invention, a nucleic acid construct according to the present invention and/or a vector according to the present invention in the manufacture of a medicament for the treatment and/or prevention of disease.

### DESCRIPTION OF THE FIGURES

FIG. 1 shows schematic representations of scFv and H9HA ectodomain fused scFv antibody expression cassettes. (A) The scFv expression cassette includes a promoter and Drosophila melanogaster immunoglobulin heavy chain binding protein (BIP) secretion signal sequence at the 5′ end followed by APCs-specific mAb variable light chain (vL), linker peptide (Gly₄Ser)₄ and variable heavy chain (vH). (B) The expression cassette includes a BIP secretion signal sequence at the 5′ end followed by H9HA gene fused with hemagglutinin trimerization signal (indicated as foldon), linked with APCs-specific mAb vL, linker peptide (Gly₄Ser)₄ and vH.

FIG. 2 shows schematic representations of recombinant hemagglutinin constructs. (A) Full length A/Chicken/Pakistan/UDL 01/2008 H9N2 hemagglutinin precursor (HA0:1-560 amino acids (aa)), HA1:19-338 aa, HA2: 339-560 aa) TM=transmembrane domain (510-550 aa) CT=cytosolic tail domain (550-560 aa). (B) Soluble A/Chicken/Pakistan/UDL 01/2008 H9N2 construct. Soluble H9HA construct was generated by removing the TM and CT domains (510-560 aa) and fusing the C-terminus of hemagglutinin to 30 aa long trimerization foldon sequence of the trimeric protein fibritin from T4.

FIG. 3 shows the results of His tag purification of the recombinant proteins. (A) His tag purification of scFv antibody. The size of the purified protein is about 30 kDa. Lane 1: Control supernatant from the untransfected cells; Lane 2: Dec205 scFv; Lane 3: CD11c scFv; Lane 4: CD83 scFv. (B) His tag purification of H9HA Foldon and H9HA Foldon-scFv. The sizes of the purified proteins are about 70 kDa and 100 kDa respectively. Lane 1: Control supernatant from the untransfected cells; Lane 2: H9HA Foldon; Lane 3: H9HA Foldon-Dec205 scFv; Lane 4: H9HA Foldon-CD11c scFv; Lane 5: H9HA Foldon-CD83 scFv.

FIG. 4 shows the results of crosslinking experiments to determine the oligomeric form of recombinant H9HA ectodomain with foldon. Lane 1: H9HA Foldon without the crosslinker bissulfosuccinimidyl suberate (BS3); Lane 2: H9HA Foldon with 10 mM BS3; Lane 3: H9HA Foldon-scFv without BS3; Lane 4: H9HA Foldon-scFv with 10 mM BS3. M=Monomer (70 kDa*100 kDa\hat{ }) D=Dimer (140 kDa*200 kDa\hat{ }) T=Trimer (210 kDa*300 kDa\hat{ })*H9HA Foldon \hat{ }H9HA Foldon-scFv.

FIG. 5 shows Table 1; the results of a hemagglutination assay to test the activity of recombinant H9HA with foldon.

FIG. 6 shows characterization of scFv and H9HA Foldon-scFv. (A) Indirect ELISA for testing the binding and detection of scFv and H9HA Foldon-scFv. (B) Detection of CD11c receptor protein from the chicken splenocytes extract by H9HA Foldon-CD11c scFv. Lane 1 represents media only control; Lane 2 represents chicken splenocytes extract. The expected molecular weight of CD11c protein is 150 kDa (shown by the arrow).

FIG. 7 shows cytokine and chemokine production by splenocytes upon stimulation with scFv antibodies. (A) Cytokines (IFNγ, IL6, IL1β, IL4 and IL18) and chemokine (CXCLi2) mRNA levels in splenocytes. Data were calculated using 2^{-ΔΔCT} approach (n-fold change compared to the media only control group) and reported as values normalised to the expression level of a housekeeping gene ribosomal phosphoprotein lateral stalk subunit PO (RPLPO1). (B) IFNγ protein level upon stimulation of splenocytes. For both (A) and (B) data were analysed by one-way ANOVA followed by Tukey's multiple comparison test. Statistical significance between test scFv and control scFv have been shown with asterisks. *p<0.05.

FIG. 8 shows cytokine and chemokine production by splenocytes upon stimulation with H9HA Foldon and H9HA Foldon-scFv. (A) Cytokines (IFNγ, IL6, IL1β, IL4 and IL18) and chemokine (CXCLi2) mRNA levels in splenocytes. Data were calculated using 2^{-ΔΔCT} approach (n-fold change compared to the media only control group) and reported as values normalised to the expression level of a housekeeping gene RPLPO1. (B) IFNγ protein level upon stimulation of splenocytes. For both panels (A) and (B) data were analysed by one-way ANOVA followed by Tukey's multiple comparison test. Statistical significance between H9HA Foldon-scFv vaccine (targeted) and H9HA Foldon vaccine (untargeted) groups have been shown with asterisks. ***p<0.001**p<0.01*p<0.05.

FIG. 9 shows Table 2; the results of a serum hemagglutination inhibition (HI) antibody titre assay. HI assay was carried out to determine HI antibody titre in the serum of the vaccinated chickens. HI titres are expressed as reciprocal of the highest dilution of antiserum that caused total inhibition of 4 units of virus hemagglutination activity. Average data is shown per (n=8). Figure legend: < indicates HI titres of less than 5; - indicates not applicable

FIG. 10 shows HA-specific IgY, IgM and IgA antibody levels in the serum of immunized chickens (35 μg dose). The HA-specific isotypes of the antibodies were determined in 200-fold diluted sera collected at day 6, 14, 21 and 28 post primary vaccination by ELISA. Data is presented as mean±SD and analysed by one-way ANOVA followed by Tukey's multiple comparison test. Statistical significance between H9HA Foldon-scFv (targeted) and H9HA Foldon (untargeted) groups has been shown with asterisks ****p<0.0001***p<0.001**p<0.01*p<0.05. The key for treatment groups is provided from left to right.

FIG. 11 shows Table 3; the virus neutralizing antibody titre measured by virus microneutralization (MNT) assay in the serum of chickens immunized with either H9HA foldon, or H9HA foldon containing CD83 scFv, CD11c or Dec205, inactivated H9N2 virus and PBS control. MNT titres are expressed as reciprocal of the highest dilution of antiserum that blocked the virus infectivity in cultured cells inoculated with 150 TCID₅₀ (50% tissue culture infective dose). Average data is shown (n=8).

FIG. 12 shows survival and average weight gain of chickens after virus challenge. Panel A) shows percentage survival between vaccinated and PBS treated control chickens challenged with H9N2 virus. Survival curves between directly infected PBS control group (bottom line at days 4-8) and directly infected or contact vaccinated groups significantly different P value=<0.05 (Log rank (Mantel-Cox) test) are shown. Panel B) shows percentage average weight gain between vaccinated and PBS treated control chickens challenged with H9N2 virus. Data was analysed by one-way ANOVA followed by Tukey's multiple comparison test. Statistical significance between the vaccinated and PBS treated control groups is shown with asterisks ****p<0.0001.

FIG. 13 shows buccal shedding profiles of vaccinated and PBS treated control chickens challenged with H9N2 virus. Panel (A) shows the buccal shedding profile of chickens on different days post infection with each chicken represented by a single point. Panel (B) shows the average buccal shedding profile of at least 6 chickens per group for directly infected birds. Panel (C) shows the average buccal shedding profile of at least 6 chickens per group for contact birds. Data is presented as mean±SD and analysed by one-way ANOVA followed by Tukey's multiple comparison test for (A, B) and by unpaired t-test for (C). For panel (B) the asterisks represent significance difference between H9HA Foldon and H9HA Foldon-CD83 scFv (direct) groups. ****p<0.0001***p<0.001**p<0.01*p<0.05. For panel (A) the key for treatment groups is provided from left to right.

FIG. 14 shows Table 4: results of a serum HI antibody titre assay. The HI assay was carried out using both vaccine virus strains UDL 01/08 and UAE/415 to determine HI antibody titre in the serum of the vaccinated chickens. HI titres are expressed as reciprocal of the highest dilution of antiserum that inhibited 4 units of virus hemagglutination activity. Average data is shown per (n=10). Figure legend < indicates HI titres of less than 5.

FIG. 15 shows nucleotide sequence SEQ ID NO: 65, which encodes amino acid SEQ ID NO: 62. SEQ ID NO: 65 comprises the following domains in order: BIP signal-H9HA ectodomain-Foldon-LINKER-Dec205 scFv-V5-His tag—nucleotide sequence.

FIG. 16 shows nucleotide sequence SEQ ID NO: 66 which encodes amino acid SEQ ID NO: 63. SEQ ID NO: 66 comprises the following domains in order: BIP signal-H9HA ectodomain-Foldon-LINKER-CD83 scFv-V5-His tag—nucleotide sequence.

FIG. 17 shows nucleotide sequence SEQ ID NO: 67 which encodes amino acid SEQ ID NO: 64. SEQ ID NO: 67 comprises the following domains in order: BIP signal-H9HA ectodomain-Foldon-LINKER-CD11c scFv-V5-His tag—nucleotide sequence.

FIG. 18 shows a schematic diagram of a genetically engineered protein comprising: at least one binding domain which is capable of binding to a cell surface protein on an avian antigen presenting cell; and at least one binding domain which is capable of binding to an antigen, wherein the genetically engineered protein is a bispecific protein comprising a binding domain capable of binding to a virus antigen.

FIG. 19 shows ELISA results demonstrating binding of bispecific scFv antibodies to avian influenza virus. IG10 is non-neutralising scFv antibody that bind to hemagglutinin protein of A/CK/Pakistan/H9N2/UDL 01/08 avian influenza virus. IG10-CD83 is bispecific antibody made by recombinantly conjugating IG10 scFv with CD83 scFv.

FIG. 20 shows ELISA results demonstrating the binding of bispecific scFv antibodies to chicken CD83 receptor protein.

FIG. 21 shows nucleotide sequence SEQ ID NO: 69 which encodes amino acid SEQ ID NO: 68. SEQ ID NO: 69 comprises the following domains in order: CD33 SIGNAL-IG10 scFv-(Glycine₄Serine)₄ linker-CD83 scFv-Ctag.

FIG. 22 Analysis of HI antibody titres in serum from chicken vaccinated with H5HA-Foldon-CD83scFv and H9HA-Foldon. HI titres were expressed as the reciprocal of the highest dilution of serum causing the total inhibition of 4 HA units of virus haemagglutination activity. Data are presented as mean±SD and analysed by one-way ANOVA followed by unpaired student t-test. ***p<0.005.

FIG. 23 Schematic representation of generation of rHVT expressing H9HA-Foldon-CD83scFv and H9HA-Foldon antigens using HDR-CRISPR/Cas9 system.

FIG. 24 The growth kinetics of rHVT-Foldon-H9HA and rHVT-H9HA-Foldon-CD83scFv. Virus titres were measured at different time points after infection with 100 pfu of each virus by (A) plaque titration and by (B) qRT-PCR of HVT SORF1 gene on DNA extracted from infected chicken fibroblast cells (CEF). The virus growth was determined by calculating HVT genome copies per 10,000 CEF cells. The data are presented as mean±SD and analysed by one-way ANOVA followed by Tukey's multiple comparison test.

FIG. 25 Analysis of HI antibody titres in serum from chicken vaccinated with rHVT-H9HA-Foldon-CD83 scFv and rHVT-H9HA-Foldon. HI titres were expressed as the reciprocal of the highest dilution of serum causing the total inhibition of 4 HA units of virus haemagglutination activity. Data are presented as mean±SD and analysed by one-way ANOVA followed by unpaired student t-test. ****p<0.0001, *p<0.05

FIG. 26 HA-specific IgY antibodies in the serum of chickens vaccinated with rHVT-H9HA-Foldon and rHVT-H9HA-Foldon-CD83 scFv. Data are presented as mean±SD and analysed by one-way ANOVA followed by Tukey's multiple comparison test. ****p<0.0001***p<0.001**p<0.01*p<0.05

FIG. 27 Analysis of virus neutralising antibody titres in serum from chickens vaccinated with targeted (rHVT-H9HA-Foldon CD83scFv) and non-targeted (rHVT-H9HA-Foldon) vaccines. Data are presented as mean±SD and analysed by one-way ANOVA followed by unpaired student t-test. **p<0.01

FIG. 28 Nucleotide sequences of constructs

FIG. 29 Amino acid sequences of constructs

FIG. 30 Schematic representation of a rNDV-H9HA-Foldon-CD83scFv. The expression cassette (H9HA-Foldon-CD83scFv) indicated as “insert” was cloned into the modified NDV genome using Not1 and Pac1 restriction sites (SEQ ID NO: 75). The expression cassette includes HA protein ectodomain (amino acid from 1-509) of H9N2 virus (A/chicken/Pakistan/827/2016, accession no. MH180417.1). The C-terminus of HA was fused with a 30 amino acid sequence of T4 fibritin trimerization signal (indicated as foldon). This followed by 248 amino acid sequence of APCs-specific CD83scFv that composed of variable light chain (vL), linker peptide (Gly₄Ser)₄ and variable heavy chain (vH). This followed by 4 amino acid (EPEA) Ctag sequence.

FIG. 31 Analysis of H9HA-specific HI antibody titres in the serum of chickens vaccinated with rNDV-H9HA-Foldon-CD83scFv and NDV control (with no H9HA insert). HI titres were expressed as the reciprocal of the highest dilution of serum causing the total inhibition of 4 HA units of H9N2 virus haemagglutination activity. Data are presented as mean±SD.

### DETAILED DESCRIPTION

The present invention provides an engineered protein which is capable of targeting a cargo to an avian antigen presenting cell. The engineered protein comprises at least one binding domain which is capable of binding to a cell surface protein on an avian antigen presenting cell. The cargo is typically capable of eliciting an immune response, such as a humoral and/or cellular immune response in an avian subject. Therefore the engineered protein according to the present invention may be used in or as an avian vaccine. Suitably, the cargo may be at least one antigenic polypeptide, such as an antigen from an avian pathogen (for example, an antigen from a virus, a bacterium or a parasite). Suitably, the cargo may be at least one binding domain which is capable of binding to at least one antigenic polypeptide, such as an antigen from an avian pathogen (for example, an antigen from a virus, a bacterium or a parasite).

An “engineered protein” as used herein refers to both genetically engineered proteins and to chemically engineered proteins.

In one aspect, the present invention provides a genetically engineered protein.

In one aspect, the present invention provides a chemically engineered protein.

In one aspect, the engineered protein is one polypeptide.

### Genetically Engineered Protein

A “genetically engineered protein” as used herein refers to a protein which has been designed and synthesised using recombinant technology.

In one aspect, the genetically engineered protein is a recombinant protein. In one aspect, the genetically engineered protein is one single recombinant protein. The genetically engineered protein may be designed, synthesized and fused using recombinant DNA technology.

Suitably, the genetically engineered protein may consist of domains which have been recombinantly fused together.

The present invention provides a genetically engineered protein comprising: at least one binding domain which is capable of binding to a cell surface protein on an avian antigen presenting cell; and
a) at least one antigenic polypeptide; or
b) at least one binding domain which is capable of binding to at least one antigenic polypeptide.

In one aspect, the genetically engineered protein is encoded by a single open reading frame.

In one aspect, the genetically engineered protein is a single recombinant protein.

In some aspects, the genetically engineered protein has not been produced by chemical conjugation.

In other words, the genetically engineered protein comprises:
a) at least one binding domain which is capable of binding to a cell surface protein on an avian antigen presenting cell; and
b) at least one antigenic polypeptide; or
c) at least one binding domain which is capable of binding to at least one antigenic polypeptide wherein a) and b) or a) and c) are joined by amino bonds.

Recombinantly engineered proteins may be more reproducible and scalable compared to other methods of producing antibody targeted vaccines. In some cases, chemical conjugation can reduce or eliminate the activity of a protein.

In one aspect, the at least one binding domain is operably linked to the at least one antigenic polypeptide; or to the at least one binding domain which is capable of binding to at least one antigenic polypeptide.

When delivered to a subject, the genetically engineered protein is capable of treating and/or preventing disease in said subject. The genetically engineered protein may be capable of eliciting a humoral and/or cellular immune response or reducing challenge pathogen load (such as viral load, bacterial load or parasitic load) when administered to a subject.

The term “operably linked” as used herein refers to a juxtaposition wherein the components described are in a relationship permitting them to function in their intended manner.

For example, in the context of vectors or expression constructs, a promoter which is capable of driving the expression of a nucleic acid sequence is operably linked to said nucleic acid sequence.

For example, in the context of an engineered protein, a binding domain capable of binding to a cell surface protein on an avian antigen presenting cell which localises a) at least one antigenic polypeptide or b) at least one binding domain which is capable of binding to at least one antigenic polypeptide on the cell surface of an avian antigen presenting cell is operably linked to a) or b) respectively.

In one aspect, the genetically engineered protein is a fusion protein.

As used herein “fusion protein” or chimeric protein refers to a protein which comprises at least two domains which are natively encoded by separate genes but have been joined together so that they are transcribed and translated to produce a single polypeptide.

The fusion protein may be encoded by a nucleic acid in which the binding domain and the antigenic polypeptide are directly or indirectly attached. An example of an indirect attachment is the provision of a suitable spacer group, such as peptide linker, between the domains.

In one aspect the binding domain and the antigenic polypeptide are linked or joined by peptide bonds.

An example of a suitable spacer is a peptide linker. Peptide linkers can be classified into three groups, flexible, rigid and cleavable. Flexible linkers are generally composed of small, non-polar or polar residues such as Gly, Ser and Thr. For example, a flexible peptide linker may be a glycine and/or serine-rich peptide. The suitable peptide linker may comprise 4-20, 4-15, 4-10, 8-20 or 8-15 amino acids. Examples of suitable peptide linkers are known in the art and include, but are not limited to, GGSGGS (SEQ ID NO: 34), SGSGSGS (SEQ ID NO: 35), GGGGSGGGGS (SEQ ID NO: 36), GSGSGSGSGS (SEQ ID NO: 37), GGSGGSGGSGGS (SEQ ID NO: 38), and GGGGSGGGGSGGGGS (SEQ ID NO: 39). More rigid linkers may include proline residues or polyproline motifs such as proline-rich sequences. For example, a suitable spacer may include a proline rich sequence (XP)ₙ where X is any amino acid, preferably Ala, Lys or Glu or a proline and glycine rich linker (PGPG)ₙ. Rigid linkers may include alpha helix-forming linkers for example, comprising the sequence of (EAAAK)ₙ.

Cleavable linkers may include cyclopeptide linkers, in vivo cleavable disulphide linkers such as LEAGCKNFFPR*SFTSCGSLE (SEQ ID NO: 33) where * indicates the cleavage site. Other cleavable linkers may include tetra-peptides such as Gly-Phe-Leu-Gly (SEQ ID NO: 70) and Ala-Leu-Ala-Leu (SEQ ID NO: 71). Cleavable linkers allow for in vivo separation of domains.

Suitably, the engineered protein may be a single recombinant protein.

Suitably, the engineered protein may be encoded by a nucleic acid construct which comprises a first polynucleotide which encodes at least one binding domain which is capable of binding to a cell surface protein on an avian antigen presenting cell; and a second polynucleotide which encodes at least one antigenic polypeptide; or a second binding domain which is capable of binding to at least one antigenic polypeptide.

In one embodiment, the present invention provides a genetically engineered protein comprising: at least one binding domain which is capable of binding to a cell surface protein on an avian antigen presenting cell; and at least one binding domain which is capable of binding to an antigen.

In some aspects, the binding domain which is capable of binding to an antigen is non-neutralising.

Typically, the at least one binding domain which is capable of binding to an antigen will be capable of binding to an antigen from an avian pathogen. Suitably, the avian pathogen may be selected from any pathogen e.g. from viruses, bacteria or parasite.

In some aspects, the antigen may be present on the surface of a virus, such as an inactivated virus. In some aspects, the antigen may be present on the surface of a bacterium, such as an inactivated bacterium. In some aspects, the antigen may be present on the surface of a parasite, such as an inactivated parasite.

The antigen may be from a virus of the Orthomyxoviridae family. For example, Avian influenza virus (AIV). The antigen may be from a virus of the Paramyxoviridae family. For example, Newcastle disease virus (NDV). The antigen may be from a virus of the Coronaviridae family. For example, Infectious bronchitis virus (IBV). The antigen may be from a virus of the Birnaviridae family. For example, Infectious bursal disease virus (IBDV). The antigen may be from a virus of the Anelloviridae family. For example, Chicken anaemia virus (CAV). The antigen may be from a virus of the Reoviridae family. For example, avian reovirus (ARV). The antigen may be from a virus of the Adenoviridae family. For example Duck Atadenovirus A. or Fowl adenoviruses (FAdV's 2, 4, 8, 11).

Suitably, the at least one binding domain which is capable of binding to a cell surface protein on an avian antigen presenting cell; and at least one binding domain which is capable of binding to an antigen are comprised in a single recombinant protein.

Suitably, at least one binding domain which is capable of binding to a cell surface protein on an avian antigen presenting cell is operably linked to the at least one binding domain which is capable of binding to an antigen.

### Antigen Presenting Cell

In one aspect, the present invention provides a genetically engineered protein comprising: at least one binding domain which is capable of binding to a cell surface protein on an avian antigen presenting cell; and
a) at least one antigenic polypeptide; or
b) at least one binding domain which is capable of binding to at least one antigenic polypeptide.

The antigen presenting cell may be any avian antigen presenting cell.

In some aspects, the genetically engineered protein comprises at least one binding domain which is capable of binding to a cell surface protein on at least one of a dendritic cell, macrophage, B cell or natural killer cell. Suitably, the binding domain may be capable of binding to a dendritic cell. Suitably, the binding domain may be capable of binding to a macrophage. Suitably, the binding domain may be capable of binding to a B cell. Suitably, the binding domain may be capable of binding to a natural killer cell.

In some aspects, the binding domain may be capable of binding to two or more or three or more or all four cells selected from a dendritic cell, macrophage, B cell or natural killer cell.

### Cell Surface Protein

In one aspect, the present invention provides an engineered protein comprising: at least one binding domain which is capable of binding to a cell surface protein on an avian antigen presenting cell; and a) at least one antigenic polypeptide; or
b) at least one binding domain which is capable of binding to at least one antigenic polypeptide.

The cell surface protein may be any avian cell surface protein as described herein.

In one aspect, the cell surface protein selected from an immunoglobulin family protein, an integrin family receptor or a C-type lectin.

CD83 is a transmembrane glycoprotein belonging to the member of immunoglobulin (Ig) superfamily. CD83 is expressed on the surface of most DCs including thymic DCs, Langerhans cells in the skin, monocytes derived DCs following culture with GM-CSF and IL4, and interdigitating reticulum cells within the spleen. CD83 is also found on the surface of macrophages, neutrophils and NK cells. CD83 has been thought to be implicated in immune response, however its function on DC and T cells remains unclear. Based on the expression profile of CD83 and its structural similarity with B7 family members (CD80/CD86), CD83 is thought to play important roles during interactions between cells of the immune system.

In one aspect, the cell surface protein may be an immunoglobulin family protein, such as CD83. In one aspect, the cell surface protein is CD83.

CD11c is a beta2 (β2) integrin expressed on variety of leukocytes including DCs, macrophages, NK cells, B and T cells. Integrins play an important role in innate immunity. They are involved in the interaction of phagocytic cells with endothelium and extracellular matrix, ingestion of complement-opsonized pathogens and cytokine production. In addition, they are also involved in the proliferation, survival and differentiation of lymphocytes during adaptive immunity.

In one aspect, the cell surface protein may be an integrin family receptor such as CD11c. In one aspect, the cell surface protein is CD11c.

C type lectin receptors (CLRs) belong to a large family of transmembrane and soluble receptors that can recognize wide variety of glycans on the pathogens in a calcium dependent manner.

Dec205 is a type I CLR which consists of a single polypeptide chain; the extracellular portion contains N-terminal cysteine-rich domain, a fibronectin type II domain and 10 carbohydrate recognition domains (CRDs). In mammals, the expression of Dec205 is primarily restricted to dendritic cells and thymic cortical epithelium, although human Dec205 can also be detected on peripheral T and B cells, natural killer (NK) cells and macrophages. In chickens, low-level expression of Dec205 has been detected in CD4+ve, CD8+ve and γδ T lymphocytes, B lymphocytes and macrophages with most expression on dendritic cells. The function of Dec205 in antigen uptake, processing and presentation has been characterized.

In one aspect, the cell surface protein may be a C-type lectin such as Dec205. In one aspect, the cell surface protein is Dec205.

### Chemically Engineered Proteins

In other aspects, the present invention provides chemically engineered proteins.

A “chemically engineered protein” as used herein refers to a protein which comprises domains which have been joined together by a non-amino group (or prosthetic group or cofactor). In other words, the protein comprises domains which are linked together without amino bonds.

The present invention provides a chemically engineered protein comprising: at least one binding domain which is capable of binding to a cell surface protein on an avian antigen presenting cell; and
a) at least one antigenic polypeptide; or
b) at least one binding domain which is capable of binding to at least one antigenic polypeptide.

In other words, the chemically engineered protein comprises:
a) at least one binding domain which is capable of binding to a cell surface protein on an avian antigen presenting cell; and
b) at least one antigenic polypeptide; or
c) at least one binding domain which is capable of binding to at least one antigenic polypeptide; wherein a) and b) or a) and c) have been chemically conjugated.

Methods for chemically conjugating proteins are known in the art and include for example the formation of a stable, covalent linkage between two or more separate proteins. Examples of chemical conjugation include the use of crosslinking reagents which typically contain two or more chemically reactive groups which will connect to the functional groups (e.g. primary amines, sulfhydrlys, carbonyls, carbohydrates or carboxylic acids) found in proteins. Crosslinkers may be homobifunctional, heterobifunctional or photoreactive.

In one aspect, the chemically engineered protein may be produced by crosslinking two or more proteins which comprise at least one binding domain which is capable of binding to a cell surface protein on an avian antigen presenting cell; and at least one antigen.

For example, a chemically engineered protein may be produced by crosslinking using a heterobifunctional crosslinking reagent to crosslink amines-to sulfhydryls e.g. using a thiolating agent followed by a Sulfo-SMCC (sulfosuccinimidyl 4-(N-maleimidomethyl)cyclohexane-1-carboxylate) reagent.

### Antigen Presenting Cell

In one aspect, the present invention provides a chemically engineered protein comprising: at least one binding domain which is capable of binding to a cell surface protein on an avian antigen presenting cell; and
a) at least one antigenic polypeptide; or
b) at least one binding domain which is capable of binding to at least one antigenic polypeptide; wherein the avian antigen presenting cell is a macrophage, B cell or natural killer cell. Suitably, the binding domain may be capable of binding to a macrophage.

Suitably, the binding domain may be capable of binding to a B cell. Suitably, the cell the binding domain may be capable of binding to a natural killer cell.

In some aspects, the binding domain may be capable of binding to a cell surface protein which is present on two or more or three or more or all four cells selected from a dendritic cell, macrophage, B cell or natural killer cells, the cell surface protein is found on dendritic cells and on macrophages.

### Cell Surface Protein

In one aspect, the present invention provides a chemically engineered protein comprising: at least one binding domain which is capable of binding to a cell surface protein on an avian antigen presenting cell; and
a) at least one antigenic polypeptide; or
b) at least one binding domain which is capable of binding to at least one antigenic polypeptide; wherein the cell surface protein is an immunoglobulin family protein or an integrin family receptor. Suitably, the cell surface protein may be an immunoglobulin family protein such as CD83. Suitably, the cell surface protein may be an integrin family receptor, such as CD11c.

In one aspect, the cell surface protein is CD83. In one aspect, the cell surface protein is CD11c.

### Aspects of Engineered Proteins

### Signal Peptide

In some aspects, the engineered protein (such as genetically engineered protein or chemically engineered protein) according to the invention comprises a signal peptide.

When a protein comprising a signal peptide is expressed in a cell, the nascent protein is directed to the endoplasmic reticulum and subsequently to the cell surface, where it is bound to the cell surface or secreted into the culture medium. Classical protein secretion may be predicted using Signal P and TargetP methods Nielsen, H., et al., (1997) Protein Eng., 10, 1-6; Emanuelsson, O., Nielsen, H., Brunak, S. and von Heijne, G. (2000) J. Mol. Biol., 300, 1005-1016), which are incorporated herein by reference.

Signal peptides are typically 16 to 30 amino acids in length and are usually found at the N-terminus of the newly synthesized protein.

An example of a signal peptide which may be used is the BIP secretion signal sequence such as SEQ ID NO: 1 below:

(SEQ ID NO: 1)
ILLAVVAFVGLSLG.

Another example of a signal peptide which may be used in the present invention is the CD33 signal peptide sequence. For example SEQ ID NO: 40 below:

(SEQ ID NO: 40)
MPLLLLLPLLWAGALAM.

Suitably, the engineered protein may comprise a signal peptide having the sequence SEQ ID NO: 1 or SEQ ID NO: 40, or variants thereof having at least 80% identity thereto which function as signal sequences.

### Solubilisation/Stability and Folding Domain

In some aspects, the engineered protein (such as genetically engineered protein or chemically engineered protein) according to the invention comprises a domain which improves solubilisation, stabilization and/or folding of the engineered protein. In particular, the domain may improve solubilisation, stabilization and/or folding of the engineered protein.

In particular, the domain may improve solubilisation, stabilization and/or folding of the antigen.

Methods for improving protein production in various systems are well known in the art and include molecular chaperones which act to preserve nascent proteins in a folding-competent conformation and prevent aggregation.

For example, various protein tags such as glutathione-S-transferase (GST), maltose-binding protein (MBP), small ubiquitin-related modifier (SUMO) and ubiquitin (UB) may be used for enhancing solubility and promoting the correct folding of recombinant proteins. Leucine zippers, GCN4 and GCN4pll may be used to facilitate oligomerization of proteins such as dimerization and trimerization.

An example of a domain which improves solubilisation, stabilization and/or folding of the engineered protein or antigen is a foldon. Suitably, the genetically engineered or chemically engineered protein may comprise a foldon sequence of the trimeric protein fibritin from bacteriophage T4, such as a 30 amino acid trimerization foldon sequence of the trimeric protein fibritin from bacteriophage T4.

The foldon domain can typically be used to improve solubilisation, stability and or folding of trimeric proteins. For example, the foldon domain may be used to improve solubilisation, stability and or folding of hemagglutinin. An exemplary sequence of a foldon domain is:

(SEQ ID NO: 2)
GSGYIPEAPRDGQAYVRKDGEWVLLSTFL

Suitably, an engineered protein according to the present invention may comprise a foldon domain having the sequence SEQ ID NO: 2, or variants thereof having at least 80% identity thereto which function to improve solubilisation, stabilization and/or folding of the engineered protein or antigen. Suitably, an engineered protein according to the present invention may comprise a foldon domain having the sequence SEQ ID NO: 2, or variants thereof having at least 15, at least 20, at least 25, at least 26, at least 27, at least 28 or at least 29 amino acids thereof.

### Binding Domain

The engineered proteins (such as genetically engineered proteins and chemically engineered proteins) according to the invention comprise at least one binding domain which is capable of binding to a cell surface protein on an avian antigen presenting cell.

In some aspects, the genetically engineered proteins and the chemically engineered proteins according to the invention further comprise at least one binding domain which is capable of binding to an antigen.

Suitably, the engineered protein may comprise at least two, at least three, at least four at least 5 or at least 6 binding domains. The binding domains may be capable of binding to two or more antigens from different strains (serotypes/genotypes) of one pathogen in order to immunize against one disease. The binding domains may be capable of binding to two or more antigens from two or more pathogens to immunize against two or more diseases. In other words, the engineered protein may be bispecific, trispecific, tetraspecific, multispecific i.e. capable of binding to two or more antigens, three or more antigens or four or more antigens.

The at least one binding domain (or antigen-binding domain) which is capable of binding to a cell surface protein on an avian antigen presenting cell is the portion of the engineered protein which recognizes and binds to a cell surface protein on an antigen presenting cell.

The at least one binding domain (or antigen-binding domain) which is capable of binding to a capable of binding to an antigen is the portion of the engineered protein which recognizes and binds to an antigen, for example an antigen on an inactivated virus, inactivated bacterium or inactivated parasite.

Various binding domains are known in the art, including those based on the antigen binding site of an antibody, an antibody fragment, antibody mimetics, and T-cell receptors.

In some aspects, it may be advantageous to use antibody fragments in the engineered proteins to improve solubility and folding.

Examples of antibody fragments capable of binding to a selected target, include Fv, scFv, F(ab′) and F(ab′)2.

In one aspect, a binding domain is based on or is a single-chain variable fragment (scFv). The scFv may comprise a variable light chain and variable heavy chains which are joined together using a linker peptide, for example using a Glycine-serine linker such as (Gly₄Ser)₄. The binding domain may be a polypeptide having an antigen binding site which comprises at least one complementarity determining region (CDR). The binding domain may comprise 3 CDRs and have an antigen binding site which is equivalent to that of a domain antibody (dAb). The binding domain may comprise 6 CDRs and have an antigen binding site which is equivalent to that of a classical antibody molecule. The remainder of the polypeptide may be any sequence which provides a suitable scaffold for the binding site and displays it in an appropriate manner for it to bind the antigen. The binding domain may be part of an immunoglobulin molecule such as a Fab, F(ab)′2, Fv, single chain Fv (scFv) fragment, Nanobody or single chain variable domain (which may be a VH or VL chain, having 3 CDRs). The binding domain may be avian. The binding domain may be chimeric.

The binding domain may comprise a binding domain which is not derived from or based on an immunoglobulin. For example, a number of “antibody mimetic” designed repeat proteins (DRPs) have been developed to exploit the binding abilities of non-antibody polypeptides. Such molecules include ankyrin or leucine-rich repeat proteins e.g. DARPins (Designed Ankyrin Repeat Proteins), Anticalins, Avimers and Versabodies.

The binding domain may “specifically bind” to the cell surface protein or antigen as defined herein. As used herein, “specifically bind” means that the binding domain binds to the cell surface protein or antigen but does not bind to other proteins, or binds at a lower affinity to other proteins.

The binding affinity between two molecules, e.g. an antigen binding domain and an antigen, may be quantified, for example, by determination of the dissociation constant (KD) e.g. by a surface plasmon resonance (SPR) method (e.g. Biacore™) The binding domain may comprise a domain which is not based on the antigen binding site of an antibody. For example the antigen binding domain may comprise a domain based on a protein/peptide which is a soluble ligand for a cell surface receptor (e.g. a soluble peptide such as a cytokine or a chemokine); or an extracellular domain of a membrane anchored ligand or a receptor for which the binding pair counterpart is expressed on the cell.

The binding domain may be based on a natural ligand of the antigen.

The binding domain may comprise an affinity peptide from a combinatorial library.

In one aspect, the binding domain capable of binding to a cell surface protein on an avian antigen presenting cell binds to CD83.

In one aspect, the binding domain capable of binding to a cell surface protein on an avian antigen presenting cell is based on the anti-CD83 antibody clone F890/GE8.

Suitably, an engineered protein according to the present invention may comprise a binding domain based on or having the amino acid sequences set forth in SEQ ID NO: 5, 6, 7 and/or SEQ ID NO: 10, 11 or 12.

Suitably, an engineered protein according to the present invention may comprise a binding domain based on or having the amino acid sequences set forth in SEQ ID NO: 4 and/or SEQ ID NO: 9 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 5, 6, 7 and/or SEQ ID NO: 10, 11 or 12.

Suitably, an engineered protein according to the present invention may comprise a binding domain encoded by the nucleotide sequences set forth in SEQ ID NO: 3 and/or SEQ ID NO: 8 or variants thereof having at least 80% identity (at least 85%, at least 90%, at least 95%, at least 97%, at least 99% identity thereto).

anti-CD83 antibody clone F890/GE8 heavy chain nucleotide sequence
(SEQ ID NO: 3)
GAGGTCCAGCTGCAACAATCTGGACCTGAGCTGGTGAAGCCTGGGGCTTCAGTGAAGATATCCTGTAA
GGCTTCTGGATACACGTTCACTGACTACTACATAAACTGGGTGAAGCAGAGCCATGGAAAGAGCCTTG
AGTGGATTGGAGATATTAATCCTACTAATGGTGATTCTACCTACAGCCAGAAGTTCAAGGGCAAGGCC
ACATTGACTGTAGACAAGTCCTCCAGCACAGCCTACATGGAGCTCCGCAGCCTGACATCTGAGGTCTC
TGCAGTCTATTACTGTGCAAGAGACTATGCTATGGACTACTGGGGTCAAGGAACCTCAGTCACCGTCT
CCTCA
anti-CD83 antibody clone F890/GE8 heavy chain amino acid sequence
(SEQ ID NO: 4)
EVQLQQSGPELVKPGASVKISCKASGYTFTDYYINWVKQSHGKSLEWIGDINPTNGDSTYSQKEKGKA
TLTVDKSSSTAYMELRSLTSEVSAVYYCARDYAMDYWGQGTSVTVSS
anti-CD83 antibody clone F890/GE8 heavy chain amino acid CDR-H1
(SEQ ID NO: 5)
DYYIN
anti-CD83 antibody clone F890/GE8 heavy chain amino acid CDR-H2
(SEQ ID NO: 6)
DINPTNGDSTYSQKFKG
anti-CD83 antibody clone F890/GE8 heavy chain amino acid CDR-H3
(SEQ ID NO: 7)
DYAMDY
anti-CD83 antibody clone F890/GE8 light chain nucleotide sequence
(SEQ ID NO: 8)
GACATTGTGATGACCCAGTCTCCATCCTCCCTGGCTGTGTCAGTCGGACAGAAGGTCACTATGAGCTG
CACGTCCAGTCAGGTCCTTTTACATAGTCCCAATCAAAAGAACTATTTGGCCTGGTACCAGCAGAAAC
CAGGACAGTCTCCTAAACTTCTGGTATACTTTGCATCCACTAGGGAATCTGGGGTCCCTGATCGCTTC
ACAGGCAGTGGATCTGGGACAGATTTCACTCTTACCATCAGCAGTGTGCAGGCTGAAGACCTGGCAGT
TTATTACTGTCAGCAACATTATAGCACTCCGCTCACGTTCGGTGCTGGGACCAAGCTGGAGCTGAAA
anti-CD83 antibody clone F890/GE8 light chain amino acid sequence
(SEQ ID NO: 9)
DIVMTQSPSSLAVSVGQKVTMSCTSSQVLLHSPNQKNYLAWYQQKPGQSPKLLVYFASTRESGVPDRF
TGSGSGTDFTLTISSVQAEDLAVYYCQQHYSTPLTFGAGTKLELK
anti-CD83 antibody clone F890/GE8 light chain amino acid CDR-L1
(SEQ ID NO: 10)
TSSQVLLHSPNQKNYLA
anti-CD83 antibody clone F890/GE8 light chain amino acid CDR-L2
(SEQ ID NO: 11)
FASTRES
anti-CD83 antibody clone F890/GE8 light chain amino acid CDR-L3
(SEQ ID NO: 12)
QQHYSTPLT

In one aspect, the binding domain is based on the anti-CD11c antibody clone 8F2.

Suitably, an engineered protein according to the present invention may comprise a binding domain based on or having the amino acid sequences set forth in SEQ ID NO: 15, 16, 17 and/or SEQ ID NO: 20, 21 or 22.

Suitably, an engineered protein according to the present invention may comprise a binding domain based on or having the amino acid sequences set forth in SEQ ID NO: 14 and/or SEQ ID NO: 19 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 15, 16, 17 and/or SEQ ID NO: 20, 21 or 22.

Suitably, an engineered protein according to the present invention may comprise a binding domain based encoded by the nucleotide sequences set forth in SEQ ID NO: 13 and/or SEQ ID NO: 18 or variants thereof having at least 80% identity (at least 85%, at least 90%, at least 95%, at least 97%, at least 99% identity thereto).

anti-CD11c antibody clone 8F2 heavy chain nucleotide sequence
(SEQ ID NO: 13)
GAGGTCCAGCTGCAGCAGTCTGGACCTGAGCTGGTAAAGCCTGGGGCTTCAGTGAAGATGTCCTGCAA
GGCTTCTGGATACACATTCACTAACTATGTTCTGCACTGGGTGAAGCAGAAGCCTGGGCAGGGCCTTG
AGTGGATTGGATATATTAATCCTTACAATGATGGTACTAAGTTCAATGAGAAGTTCAAAGGCAAGGCC
ACACTGACTTCAGACACATCCTCCAGCACAGCCTTCATGGAACTCAGCAGCCTGACCTCTGAGGACTC
TGCGGTCTATTACTGTGCAAGAGGAGATAATCTACGGCCCTACTACTTTGACTACTGGGGCCAAGGCA
CCACTCTCACAGTCTCCTCA
anti-CD11c antibody clone 8F2 heavy chain amino acid sequence
(SEQ ID NO: 14)
EVQLQQSGPELVKPGASVKMSCKASGYTFTNYVLHWVKQKPGQGLEWIGYINPYNDGTKENEKFKGKA
TLTSDTSSSTAFMELSSLTSEDSAVYYCARGDNLRPYYFDYWGQGTTLTVSS
anti-CD11c antibody clone 8F2 heavy chain amino acid CDR-H1
(SEQ ID NO: 15)
NYVLH
anti-CD11c antibody clone 8F2 heavy chain amino acid CDR-H2
(SEQ ID NO: 16)
YINPYNDGTKFNEKFKG
anti-CD11c antibody clone 8F2 heavy chain amino acid CDR-H3
(SEQ ID NO: 17)
GDNLRPYYEDY
anti-CD11c antibody clone 8F2 light chain nucleotide sequence
(SEQ ID NO: 18)
CAAATTGTTCTCACCCATTCTCCAGCAATCATGTCTGCATCTCCAGGGGAGAAGGTCACCATGACCTG
CAGTGCCAGCTCAAGTGTAAGTTTCATGTACTGGTACCAGCAGAAGCCAGGATCCTCCCCCCGACTCC
TGCTTTATGACACATCCAGCCTGTCTTCTGGAGTCCCTGTTCGCTTCAGTGGCAGTGGCTCTGGGACC
TCTTACTCTCTCACAATCAGCCGAATGGAGGCTGAAGATGCTGCCACTTATTACTGCCAGCAGTGGAG
TCGTTACCCACCGACGTTCGGTGGAGGCACCAAGCTGGAAATCAAA
anti-CD11c antibody clone 8F2 heavy chain amino acid sequence
(SEQ ID NO: 19)
QIVLTHSPAIMSASPGEKVTMTCSASSSVS FMYWYQQKPGSSPRLLLYDTSSLSSGVPVRFSGSGSGT
SYSLTISRMEAEDAATYYCQQWSRYPPT FGGGTKLEIK
anti-CD11c antibody clone 8F2 heavy chain amino acid CDR-L1
(SEQ ID NO: 20)
SASSSVSEMY
anti-CD11c antibody clone 8F2 heavy chain amino acid CDR-L2
(SEQ ID NO: 21)
DTSSLSS
anti-CD11c antibody clone 8F2 heavy chain amino acid CDR-L3
(SEQ ID NO: 22)
QQWSRYPPT

Suitably, the binding domain may be produced against or may recognise carbohydrate recognition domains 4, and/or 5, and/or 6 of the chicken Dec205 receptor.

In one aspect, the binding domain is not produced against or does not recognise carbohydrate recognition domain 2 of chicken Dec205 receptor.

In one aspect, the binding domain is based on the anti-Dec205 antibody clone F887/AD6 Suitably, an engineered protein according to the present invention may comprise a binding domain based on or having the amino acid sequences set forth in SEQ ID NO: 25, 26, 27 and/or SEQ ID NO: 30, 31 or 32.

Suitably, an engineered protein according to the present invention may comprise a binding domain based on or having the amino acid sequences set forth in SEQ ID NO: 24 and/or SEQ ID NO: 29 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 25, 26, 27 and/or SEQ ID NO: 30, 31 or 32.

Suitably, an engineered protein according to the present invention may comprise a binding domain encoded by the nucleotide sequences set forth in SEQ ID NO: 23 and/or SEQ ID NO: 28 or variants thereof having at least 80% identity (at least 85%, at least 90%, at least 95%, at least 97%, at least 99% identity thereto).

anti-DEC205 antibody clone F887/AD6 heavy chain nucleotide sequence
(SEQ ID NO: 23)
GAGGTGCAACTGGTGGAGTCTGGGGGAGACTTAGTGAAGCCTGGAGGGTCCCTGAAACTCTC
CTGTGCAGCCTCTGGATTCACTTTCAGTAGCTATGGCATGTCTTGGGTTCGCCAGACTCCAG
ACAAGAGGCTGGAGTGGGTCGCAACCATTAGTAGTGGTGGTAGTTACACCTACTATCCAGAC
AGTGTGAAGGGGCGATTCACCATTTCCAGAGACAATGCCAAGAACATCCTGTATCTGCAAAT
GAGCAGTCTGAAGTCTGAAGACACAGCCATGTATTACTGTGCAAGACTTTCAACCTGGGACT
GGTACTTCGATGTCTGGGGCACAGGGACCACGGTCACCGTCTCCTCA
anti-DEC205 antibody clone F887/AD6 heavy chain amino acid sequence
(SEQ ID NO: 24)
EVQLVESGGDLVKPGGSLKLSCAASGFTFSSYGMSWVRQTPDKRLEWVATISSGGSYTYYPD
SVKGRFTISRDNAKNILYLQMSSLKSEDTAMYYCARLSTWDWYFDVWGTGTTVTVSS
anti-DEC205 antibody clone F887/AD6 heavy chain amino acid CDR-H1
(SEQ ID NO: 25)
SYGMS
anti-DEC205 antibody clone F887/AD6 heavy chain amino acid CDR-H2
(SEQ ID NO: 26)
SSGGSYTYYPDSVKGRF
anti-DEC205 antibody clone F887/AD6 heavy chain amino acid CDR-H3
(SEQ ID NO: 27)
LSTWDWYFDV
anti-DEC205 antibody clone F887/AD6 light chain nucleotide sequence
(SEQ ID NO: 28)
GAAATTGTGCTCACCCAGTCTCCAGCACTCATGGCTGCATCTCCAGGGGAGAAGGTCACCAT
CACCTGCAGTGTCAGCTCAAGTATAAGTTCCGGCAACTTTCACTGGTACCAGCAGAAGTCAG
GAACCTCCCCCAAACTCTGGATTTATGGCACATCCAACCTGGCTTCTGGAGTCCCTGTTCGC
TTCAGTGGCAGTGGATCTGGGACCTCTTATTCTCTCACAATCAGCAGCATGGAGGCTGAAGA
TGCTGCCACTTATTACTGTCAACAGTGGAGTAGTTACCCATTCACGTTCGGCTCGGGGACAA
AGTTGGAAATAAAA
anti-DEC205 antibody clone F887/AD6 light chain amino acid sequence
(SEQ ID NO: 29)
EIVLTQSPALMAASPGEKVTITCSVSSSISSGNFHWYQQKSGTSPKLWIYGTSNLASGVPVR
FSGSGSGTSYSLTISSMEAEDAATYYCQQWSSYPFTFGSGTKLEIK
anti-DEC205 antibody clone F887/AD6 amino acid CDR-L1
(SEQ ID NO: 30)
SVSSSISSGNFH
anti-DEC205 antibody clone F887/AD6 amino acid CDR-L2
(SEQ ID NO: 31)
GTSNLAS
anti-DEC205 antibody clone F887/AD6 amino acid CDR-L3
(SEQ ID NO: 32)
QQWSSYPFT

In one aspect the present invention provides an antibody or antigen-binding fragment thereof which binds to CD83, such as avian CD83, in particular to poultry and/or chicken CD83.

In one aspect the present invention provides an antibody or antigen-binding fragment thereof having the CDRs set forth in SEQ ID NO: 5, 6, 7 and/or SEQ ID NO: 10, 11 or 12.

Suitably, the antibody may comprise sequences set forth in SEQ ID NO: 4 and/or SEQ ID NO: 9 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 5, 6, 7 and/or SEQ ID NO: 10, 11 or 12.

Suitably, the antibody may comprise a binding domain encoded by the nucleotide sequences set forth in SEQ ID NO: 3 and/or SEQ ID NO: 8 or variants thereof having at least 80% identity (at least 85%, at least 90%, at least 95%, at least 97%, at least 99% identity thereto). In one aspect the present invention provides an antibody or antigen-binding fragment thereof which binds to CD11c, such as avian CD11c, in particular to poultry and/or chicken CD11c. In a further aspect the present invention provides an antibody or antigen-binding fragment thereof having the CDRs set forth in SEQ ID NO: 15, 16, 17 and/or SEQ ID NO: 20, 21 or 22.

Suitably, the antibody may comprise sequences set forth in SEQ ID NO: 14 and/or SEQ ID NO: 19 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 15, 16, 17 and/or SEQ ID NO: 20, 21 or 22.

Suitably, the antibody may comprise a binding domain based encoded by the nucleotide sequences set forth in SEQ ID NO: 13 and/or SEQ ID NO: 18 or variants thereof having at least 80% identity (at least 85%, at least 90%, at least 95%, at least 97%, at least 99% identity thereto).

In one aspect the present invention provides an antibody or antigen-binding fragment thereof which binds to Dec205, such as avian Dec205, in particular to poultry and/or chicken Dec205.

In a further aspect the present invention provides an antibody or antigen-binding fragment thereof having the CDRs set forth in SEQ ID NO: 25, 26, 27 and/or SEQ ID NO: 30, 31 or 32.

Suitably, the antibody may comprise the amino acid sequences set forth in SEQ ID NO: 24 and/or SEQ ID NO: 29 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 25, 26, 27 and/or SEQ ID NO: 30, 31 or 32.

Suitably, the antibody may comprise a binding domain encoded by the nucleotide sequences set forth in SEQ ID NO: 23 and/or SEQ ID NO: 28 or variants thereof having at least 80% identity (at least 85%, at least 90%, at least 95%, at least 97%, at least 99% identity thereto).

In one aspect, the binding domain capable of binding to an antigen binds to hemagglutinin of an avian influenza virus, such as hemagglutinin antigen of H9N2 avian influenza virus.

In one aspect, the binding domain is based on the anti-CD83 antibody clone F955/IG10.

Suitably, the engineered protein according to the present invention may comprise a binding domain based on or having the amino acid sequences set forth in SEQ ID NO: 44, 45, 46 and/or SEQ ID NO: 49, 50 or 51.

Suitably, the engineered protein according to the present invention may comprise a binding domain based on or having the amino acid sequences set forth in SEQ ID NO: 43 and/or SEQ ID NO: 48 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 44, 45, 46 and/or SEQ ID NO: 49, 50 or 51.

Suitably, the engineered protein according to the present invention may comprise a binding domain encoded by the nucleotide sequences set forth in SEQ ID NO: 42 and/or SEQ ID NO: 47 or variants thereof having at least 80% identity (at least 85%, at least 90%, at least 95%, at least 97%, at least 99% identity thereto).

anti-hemagglutinin antibody clone F955/IG10 heavy chain nucleotide sequence
(SEQ ID NO: 42)
GAGGTTCAGCTGCAGCAGTCTGTGGCAGAGCTTGTGAGGCCAGGGGCCTCAGTCAAGTTGTCCTGCAC
AGCTTCTGGCTTCAACATTAAAAACACCTATATGCACTGGGTGAAGCAGAGGCCTGAACAGGGCCTGG
AGTGGATTGGAAGGATTGATCCTGCGAATGGTAATACTAGGTATGCCCCGAAGTTCCAGGGCAAGGCC
ACTATAACTGCAGACACATCCTCCAACACAGCCTACCTGCAGCTCAGCAGCCTGACATCTGAGGACAC
TGCCATCTATTACTGTGCCCGTTATTACTTCGGTCCTGACTACTGGGGCCAAGGCACCACTCTCACAG
TCTCCTCA
anti-hemagglutinin antibody clone F955/IG10 heavy chain amino acid sequence
(SEQ ID NO: 43)
EVQLQQSVAELVRPGASVKLSCTASGFNIKNTYMHWVKQRPEQGLEWIGRIDPANGNTRYAPKFQGKA
TITADTSSNTAYLQLSSLTSEDTAIYYCARYYFGPDYWGQGTTLTVSS
anti-hemagglutinin antibody clone F955/IG10 heavy chain amino acid CDR-H1
(SEQ ID NO: 44)
NTYMH
anti-hemagglutinin antibody clone F955/IG10 heavy chain amino acid CDR-H2
(SEQ ID NO: 45)
RIDPANGNTRYAPKFQG
anti-hemagglutinin antibody clone F955/IG10 heavy chain amino acid CDR-H3
(SEQ ID NO: 46)
YYFGPDY
anti-hemagglutinin antibody clone F955/IG10 light chain nucleotide sequence
(SEQ ID NO: 47)
GACATCCTGATGACCCAATCTCCATCCTCCATGTCTGTATCTCTGGGAGACACAGTCATCATCACTTG
CCATGCAAGTCAGGGCATTAGCAGTAATATAGGGTGGTTGCAGCAGAAACCAGGGAAATCATTTAAGG
GCCTGATCTATCATGCAACCAACTTGGAAGATGGAGTTCCATCAAGGTTCAGTGGCGGTGGATCTGGA
GCAGATTATTCTCTCACCATCAGCAGCCTGGAATCTGAAGATTTTGCAGACTATTACTGTGTACAGTA
TGGTCAGTTTCCATTCACGTTCGGCTCGGGGACAAAGTTGGAAATAAAA
anti-hemagglutinin antibody clone F955/IG10 light chain amino acid sequence
(SEQ ID NO: 48)
DILMTQSPSSMSVSLGDTVIITCHASQGISSNIGWLQQKPGKSFKGLIYHATNLEDGVPSRFSGGGSG
ADYSLTISSLESEDFADYYCVQYGQFPFTFGSGTKLEIK
anti-hemagglutinin antibody clone F955/IG10 light chain amino acid CDR-L1
(SEQ ID NO: 49)
HASQGISSNIG
anti-hemagglutinin antibody clone F955/IG10 light chain amino acid CDR-L2
(SEQ ID NO: 50)
HATNLED
anti-hemagglutinin antibody clone F955/IG10 light chain amino acid CDR-L3
(SEQ ID NO: 51)
VQYGQFPFT

In one aspect, the binding domain capable of binding to an antigen binds to hemagglutinin of an avian influenza virus, such as hemagglutinin antigen of H9N2 avian influenza virus.

In one aspect, the binding domain is based on the anti-CD83 antibody clone F955/HD8.

Suitably, the engineered protein according to the present invention may comprise a binding domain based on or having the amino acid sequences set forth in SEQ ID NO: 54, 55, 56 and/or SEQ ID NO: 59, 60 or 61.

Suitably, the engineered protein according to the present invention may comprise a binding domain based on or having the amino acid sequences set forth in SEQ ID NO: 53 and/or SEQ ID NO: 58 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 54, 55, 56 and/or SEQ ID NO: 59, 60 or 61.

Suitably, the engineered protein according to the present invention may comprise a binding domain encoded by the nucleotide sequences set forth in SEQ ID NO: 52 and/or SEQ ID NO: 57 or variants thereof having at least 80% identity (at least 85%, at least 90%, at least 95%, at least 97%, at least 99% identity thereto).

anti-hemagglutinin antibody clone F955/HD8 heavy chain nucleotide sequence
(SEQ ID NO: 52)
GAGGTTCAGCTGCAGCAGTCTGTGGCAGAGCTTGTGAGGCCAGGGGCCTCAGTCAAGTTGTCCTGCAC
AGCTTCTGGCTTCAACATTAAAAACACCTATATGCACTGGGTGAAGCAGAGGCCTGAACAGGGCCTGG
AGTGGATTGGAAGGATTGATCCTGCGAATGGTAATACTAGATATGCCCCGAAATTCCAGGGCAAGGCC
ACTATAACTGCAGACACATCCTCCAACACAGCCTACCTGCAGCTCAGCAGCCTGACATCTGACGACAC
TGCCATCTATTACTGTGGTAGGACAGAGTTCAGGAATGCTATGGACTACTGGGGTCAAGGAACCTCAG
TCACCGTCTCCTCA
anti-hemagglutinin antibody clone F955/HD8 heavy chain amino acid sequence
(SEQ ID NO: 53)
EVQLQQSVAELVRPGASVKLSCTASGFNIKNTYMHWVKQRPEQGLEWIGRIDPANGNTRYAPKFQGKA
TITADTSSNTAYLQLSSLTSDDTAIYYCGRTEFRNAMDYWGQGTSVTVSS
anti-hemagglutinin antibody clone F955/HD8 heavy chain amino acid CDR-H1
(SEQ ID NO: 54)
NTYMH
anti-hemagglutinin antibody clone F955/HD8 heavy chain amino acid CDR-H2
(SEQ ID NO: 55)
RIDPANGNTRYAPKFQG
anti-hemagglutinin antibody clone F955/HD8 heavy chain amino acid CDR-H3
(SEQ ID NO: 56)
TEFRNAMDY
anti-hemagglutinin antibody clone F955/HD8 light chain nucleotide sequence
(SEQ ID NO: 57)
GACATCCAGATGACTCAGTCTCCAGCCTCCCTATCTCCATCTGTGGGAGAAACTGTCACCATGACATG
TCGAGCAAGTGAGAATATTTACAGTAATTTAGCATGGTATCAGCAGAAACAGGGAAAATCTCCTCAGC
TCCTGGTCTATGCTGCAACAAACTTAGCAGATGGTGTGCCATCAAGGTTCAGTGGCAGTGGATCAGGC
ACACAGTTTTCTCTGAAGATCAACAGCCTGCAGCCTGAAGATTTTGGGAATTATTACTGTCAACATTT
TTATAATACTCCGTACACGTTCGGAGGGGGGACCAAGCTGGAAATAAAA
anti-hemagglutinin antibody clone F955/HD8 light chain amino acid sequence
(SEQ ID NO: 58)
DIQMTQSPASLSPSVGETVTMTCRASENIYSNLAWYQQKQGKSPQLLVYAATNLADGVPSRFSGSGSG
TQFSLKINSLQPEDFGNYYCQHFYNTPYTFGGGTKLEIK
anti-hemagglutinin antibody clone F955/HD8 light chain amino acid CDR-L1
(SEQ ID NO: 59)
RASENIYSNLA
anti-hemagglutinin antibody clone F955/HD8 light chain amino acid CDR-L2
(SEQ ID NO: 60)
AATNLAD
anti-hemagglutinin antibody clone F955/HD8 light chain amino acid CDR-L3
(SEQ ID NO: 61)
QHFYNTPYT

In one aspect the present invention provides an antibody or antigen-binding fragment thereof which binds to hemagglutinin of an avian influenza virus, such as hemagglutinin antigen of H9N2 avian influenza virus.

In one aspect the present invention provides an antibody or antigen-binding fragment thereof having the CDRs set forth in SEQ ID NO: 44, 45, 46 and/or SEQ ID NO: 49, 50 or 51.

Suitably, the antibody may comprise sequences set forth in SEQ ID NO: 43 and/or SEQ ID NO: 48 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 44, 45, 46 and/or SEQ ID NO: 49, 50 or 51.

Suitably, the antibody may comprise a binding domain encoded by the nucleotide sequences set forth in SEQ ID NO: 42 and/or SEQ ID NO: 47 or variants thereof having at least 80% identity (at least 85%, at least 90%, at least 95%, at least 97%, at least 99% identity thereto).

In one aspect the present invention provides an antibody or antigen-binding fragment thereof having the CDRs set forth in SEQ ID NO: 54, 55, 56 and/or SEQ ID NO: 59, 60 or 61.

Suitably, the antibody may comprise sequences set forth in SEQ ID NO: 53 and/or SEQ ID NO: 58 or variants thereof having at least 80% identity (at least 85%, at least 90%, at least 95%, at least 97%, at least 99% identity thereto). Suitably, the variant may comprise at least one, at least two, at least 3, at least 4, at least 5 at least 6 of the CDRs set forth in SEQ ID NO: 54, 55, 56 and/or SEQ ID NO: 59, 60 or 61.

Suitably, the antibody may comprise a binding domain encoded by the nucleotide sequences set forth in SEQ ID NO: 52 and/or SEQ ID NO: 57 or variants thereof having at least 80% identity (at least 85%, at least 90%, at least 95%, at least 97%, at least 99% identity thereto).

### Cell Surface Proteins

The engineered proteins (such as genetically engineered proteins and chemically engineered proteins) according to the invention comprise at least one binding domain which is capable of binding to a cell surface protein on an avian antigen presenting cell.

As used herein, the “cell surface protein” refers to a protein which is expressed on the surface of a cell. In other words, at least part of the protein is exposed to the extracellular space.

The cell surface protein may be a plasma membrane protein which has at least part of a domain exposed to the extracellular space or the exoplasmic surface of the plasma membrane.

The cell surface tissue antigen may be an integral (or intrinsic) membrane protein. Integral membrane proteins are permanently attached to the membrane and have one or more domains which are embedded in the phospholipid bilayer. Examples of integral membrane proteins include transporters, channels, receptors and cell adhesion proteins.

The cell surface tissue antigen may be a transmembrane protein. Transmembrane proteins span the lipid bilayer. Transmembrane proteins may be single or multi-pass membrane proteins. For example, the transmembrane protein may be a member of the immunoglobulin superfamily. The cell surface protein may be an integral monotropic protein, associated with one side of the lipid bilayer and do not span the lipid bilayer.

The cell surface protein may be a peripheral membrane protein. Peripheral membrane proteins do not interact with the hydrophobic core of the phospholipid bilayer. Peripheral membrane proteins are typically bound to the membrane indirectly by interactions with integral membrane proteins or directly by interactions with lipid polar head groups.

Peripheral proteins may be localized to the outer surface of the plasma membrane. The cell surface protein may be a peripheral exoplasmic membrane protein.

The cell surface tissue antigen may be anchored to the plasma membrane e.g. covalently attached to lipids embedded within the cell membrane (such as via a glycosylphosphatidylinositol (GPI) anchor).

The cell surface membrane protein may be a GPI-anchored protein.

Numerous methods exist for determining the subcellular localisation of proteins and include for example: electron microscopy; confocal microscopy using co-localisation with known membrane proteins; immuno-fluorescence; and flow cytometry with fluorescently tagged antibodies.

The engineered proteins (such as genetically engineered proteins and chemically engineered proteins) according to the invention may comprise at least one binding domain which binds to a cell surface protein selected from CD83, CD11c, Dec205, BU-1, CD28, CD40, CD14, CD80, CD86, MRC1L, CD25, CD45, MHVII or CD44.

The genetically engineered proteins and the chemically engineered proteins according to the invention may comprise at least one binding domain which binds to a cell surface protein listed in Table 5 below:

TABLE 5
Cell surface proteins on avian antigen presenting cells for targeted delivery
of antigens by binding domains such as scFv monoclonal antibodies surface

| | Cell surface | Expression on antigen presenting | |
|---|---|---|---|
| | protein | cells | Exemplary antibody |
| 1 | BU-1 | B cells | Southern Biotech, Cat |
| | | | no: 8395-02, Clone: AV20 |
| 2 | CD28 | T cells | Southern Biotech, Cat no: |
| | | | 8260-01, Clone: AV7 |
| 3 | CD40 | B-cells, Macrophages, Monocytes | Bio-Rad Cat no: MCA2836, |
| | | | Clone AV79 |
| 4 | CD14 | Macrophage | Bio-Rad, Cat no: MCA5926GA, |
| | | | Clone: AV141 |
| 5 | CD80 | B cells, DCs, Macrophages | Bio-Rad Cat no: MCA2837 |
| 6 | CD86 | B cells, DCs, Macrophages | Bio-Rad Cat no: MCA2838, |
| | | | Clone: IAH/F853/AG2 |
| 7 | CD25 | T cells, Monocytes, Macrophages | Bio-Rad, Cat no: MCA5925GA, |
| | | | Clone: AV142 |
| 8 | CD45 | B cells, T cells | Bio-Rad, Cat no: MCA2413GA, |
| | | | Clone UM16-6 |
| 9 | MHCII | DCs, Macrophages, B cells | Southern Biotech, Cat no: |
| | | | 8350-01, Clone: 2G11 |
| 10 | CD44 | B cells, T cells, Monocytes | Southern Biotech, Cat no: |
| | | | 8400-01, Clone: AV6 |

### Antigen

In some aspects, the engineered proteins (such as genetically engineered proteins and chemically engineered proteins) according to the invention comprise at least one antigenic polypeptide.

In one aspect, the antigenic polypeptide is at least part of the antigen.

In other words, the engineered protein may comprise the amino acid sequence of at least part of the antigen which is antigenic. For example, the engineered protein may comprise part of a domain or of one or more domains of the antigen.

In one aspect, the engineered protein comprises the entire antigen.

Typically, the antigen or part thereof or antigenic polypeptide will be capable of inducing specific neutralizing antibodies against a pathogen e.g. virus, bacteria or parasite.

Without wishing to be bound by theory, the antigenic polypeptide will be recognised by the subject's immune system and will elicit a humoral and/or cellular immune response.

Suitably, the at least one antigenic polypeptide may be from an avian pathogen.

As used herein, “antigen or antigenic polypeptide from an avian pathogen” refers to an antigen or antigenic polypeptide from a pathogen found in an avian subject. For example the antigen or antigenic polypeptide may be associated with disease in the avian subject and may not be expressed or may be expressed at a low level in healthy avian subjects.

Suitably, the antigen may be expressed at a level which is at least 20% higher, at least 30% higher, at least 40% higher, at least 50% higher, at least 60% higher, at least 70% higher, at least 80% higher, at least 90% higher than in a corresponding healthy avian subject.

Suitably, the antigen may be disease-specific.

As used herein “disease-specific” means that the antigen is not expressed or is expressed at a lower amount in a healthy avian subject. The level of expression may be calculated using methods known in the art, for example ELISA. A comparison may be made between samples taken from a subject known to be infected with a disease and subjects known to be healthy.

The antigen may be from any pathogen. In one aspect the antigen is a viral antigen. In another aspect, the antigen is a bacterial antigen.

For example, the viral antigen may be an antigen from AIV, NDV, aMPV, IBV, IBD, IBDV), CAV, ARV, or AdV.

In one aspect, the antigenic polypeptide may comprise or consist of an immunogenic peptide epitope. The antigenic polypeptide may compromise or consist of a peptide immunogenic epitope from an avian pathogen.

An “immunogenic peptide epitope” as used herein refers to a peptide which is recognised by either a T cell receptor (TCR) or a B cell receptor (BCR)/antibody.

“Peptides” are short chains of two or more amino acids. Typically peptides consist of up to around twenty amino acids.

Suitably, the immunogenic peptide epitope may be a T cell epitope or a B cell epitope.

In one aspect the immunogenic peptide epitope is a T cell epitope.

In an adaptive immune response, T cells are capable of recognising internal epitopes of a protein antigen. APCs take up protein antigens and degrade them into short peptide fragments. A peptide may bind to a major histocompatibility complex (MHC class II) inside the cell and be carried to the cell surface. When presented at the cell surface in conjunction with an MHC molecule, the peptide may be recognised by a T cell (via the TCR), in which case the peptide is a T cell epitope.

Suitably, an immunogenic peptide epitope may be capable of being recognised by a TCR when presented in the context of a MHC. Peptides that bind to MHC class I are typically 7 to 13, more usually 8 to 10 amino acids in length. The binding of the peptide is stabilised at its two ends by contacts between atoms in the main chain of the peptide and invariant sites in the peptide-binding groove of all MHC class I molecules. There are invariant sites at both ends of the groove which bind the amino and carboxy termini of the peptide. Variations in peptide length are accommodated by a kinking in the peptide backbone, often at proline or glycine residues that allow flexibility.

Peptides which bind to MHC class II molecules are typically between 8 and 20 amino acids in length, more usually between 10 and 17 amino acids in length, and can be longer (for example up to 40 amino acids). These peptides lie in an extended conformation along the MHC II peptide-binding groove which (unlike the MHC class I peptide-binding groove) is open at both ends. The peptide is held in place mainly by main-chain atom contacts with conserved residues that line the peptide-binding groove.

A T cell epitope may thus be a peptide derivable from an antigen which is capable of binding to the peptide-binding groove of an MHC molecule and being recognised by a T cell.

The minimal epitope is the shortest fragment derivable from an epitope, which is capable of binding to the peptide-binding grove of an MHC class I or II molecule and being recognised by a T cell. For a given immunogenic region, it is typically possible to generate a “nested set” of overlapping peptides which act as epitopes, all of which contain the minimal epitope but differ in their flanking regions.

Thus, it is possible to identify the minimal epitope for a particular MHC molecule: T cell combination by measuring the response to truncated peptides. For example, if a response is obtained to the peptide comprising residues 1-15 in the overlapping library, sets which are truncated at both ends (i.e. 1-14, 1-13, 1-12 etc. and 2-15, 3-15, 4-15 etc.) can be used to identify the minimal epitope.

Bioinformatics methods for predicting T cell epitopes from a protein are known in the art and include, but are not limited to, EpiDOCK, MotifScan, Rankpep, SYFPEITHI, MAPPP, PREDIVAC, PEPVAC, EPISOPT, Vaxign, MHCPred, EpiTOP, BIMAS, TEPITOPE, Propred, EpiJen, IEDB-MHCI, IEDB-MHCII, MULTIPRED2, MHC2PRED, NetMHC, NetMHCII, NetMHCpan, NetMHCllpan, nHLApred, SVMHC, SVRMHC, NetCTL and WAPP.

In one aspect the immunogenic peptide epitope is a B cell epitope. A B cell epitope refers to a peptide which is capable of binding to a B cell receptor (BCR)/antibody. B cell epitopes are generally divided into two categories, conformational epitopes and linear epitopes, based on their structure and interaction with the antibody. A conformational epitope is formed by the 3D conformation adopted by the interaction of discontinuous amino acid residues. In contrast, a linear epitope is formed by the 3D conformation adopted by the interaction of contiguous amino acid residues.

Suitably, the peptide epitope may be a linear B cell epitope when provided in the linked antigenic peptide construct.

Bioinformatics methods for predicting B cell epitopes from a protein are known in the art and include, but are not limited to, linear B cell epitope predictors such as PEOPLE, BepiPred, ABCpred, LBtope, BCPREDS and SVMtrip and conformational B cell epitope predictors such as CEP, DiscoTope, ElliPro, PEPITO, SEPPA, EPITOPIA, EPSVR, EPIPRED, PEASE, MIMOX, PEPITOPE, EpiSearch, MIMOPRO and CBTOPE.

The present engineered protein may comprise at least one B cell epitope and at least one T cell epitope as defined herein.

Suitably, the antigenic polypeptide may comprise at least one immunogenic B cell epitope. The peptide epitope(s) may independently be at least 7, at least 10, at least 15, at least 20, at least 30, at least 40, at least 50 or at least 100 amino acids in length.

The peptide epitope(s) may independently be about 7 to about 100, about 7 to about 50, or about 7 to about 20 amino acids in length.

The antigenic polypeptide induces an immune response when administered to a subject.

The antigenic polypeptide may induce a cytotoxic T cell response and/or a humoral immune response in a subject. Preferably, the antigenic polypeptide may induce a memory humoral immune response in a subject.

Methods for determining if a peptide is immunogenic are known in the art and include, for example, immune cell activation assays using CD4+ and/or CD8+ T cells or B cells.

Suitable markers for activation may include T cell proliferation and/or expression of cytokines (e.g. IFNγ, IL6, IL1β, IL4 and CxCLi2 (IL8) using methods such as quantitative PCT, ELISA and/or ELISpot.

T cell epitopes may be determined using the above assays, and/or MHC binding assays. Suitably, B cell epitopes may be identified from epitopes bound by antibodies isolated from a subject previous infected/recovered from a pathogen infection and/or previously vaccinated with an antigen from the pathogen. Methods for determining the epitope bound by an antibody (i.e. a B cell epitope) include, but are not limited to, X-ray crystallography, cryogenic electron microscopy, array-based oligo-peptide screening, site-directed mutagenesis mapping, high-throughput mutagenesis mapping, hydrogen-deuterium exchange, cross-linking-coupled mass spectrometry.

For example, the antigen may be an antigen from AIV, NDV, aMPV, IBV, IBDV, CAV or ARV.

Suitably, the antigen may be: hemagglutinin from avian influenza (for example, GenBank accession number: ACP50708.1, HA1: 19-349 and HA2: 1-174); the fusion protein (F) protein from NDV (for example GenBank accession number: AAK55550.1); Haemagglutinin-neuraminidase (HN) from NDV (for example GenBank accession number: MH614933.1); VP2 protein from IBDV (for example GenBank accession number: KX827589.1); spike protein from IBV (for example GenBank accession number: AAA66578.1); or VP1 and/or VP2 protein from CAV (for example GenBank accession number: AQM56826.1 and AF313470.1).

In one aspect, the antigen is an avian influenza antigen and the vaccine of the invention treats and/or prevents avian influenza. For example, the antigen may be hemagglutinin.

In some aspects, the hemagglutinin may be synthetically produced using a consensus sequence. The hemagglutinin may be from any subtype. For example, the hemagglutinin may be selected from any of H1, H2, H3, H4, H5, H6, H7, H8, H9, H10, H11, H12, H13, H14, H15, H16, H17 or H18, suitably, the hemagglutinin may be selected from an avian influenza virus.

In some aspects, the antigen may have about 90%, (such as about 95%, such as about 96%, such as about 97%, such as about 98%) amino acid sequence similarity to the hemagglutinin ectodomain of A/Chicken/Pakistan/UDL 01/2008 H9N2 virus (GenBank accession number: ACP50708.1, HA1: 19-349 and HA2: 1-174) virus or the H5HA antigen of avian influenza H5N8 virus strain (A/duck/Egypt/SS19/2017, accession no. MH893738.1). Suitably, the antigen may be soluble. Suitably, the antigen may be a secreted protein.

In some aspects, the antigen is engineered to improve solubility and/or secretion. For example, the antigen may be engineered to remove any transmembrane domains which are typically present. The antigen may be engineered to comprise a signal peptide which allows secretion from cells in the subject.

In some aspects, the genetically engineered protein or chemically engineered protein may comprise an ectodomain of the H9HA gene that lacks both the hemagglutinin gene signal peptide and the transmembrane domain, replaced with a 30 amino acid trimerization foldon sequence of the trimeric protein fibritin from bacteriophage T4.

Suitably, the H9HA ectodomain for use in the present invention may comprise the amino acid sequence set forth in SEQ ID NO: 41:

(SEQ ID NO: 41)
DKICIGHQSTNSTETVDTLTETNVPVTHAKELLHTEHNGMLCATNLGHP
LILDTCTIEGLIYGNPSCDLLLGGREWSYIVERPSAVNGTCYPGNVENL
EELRTLFSSSSSYQRIQIFPDTIWNVTYTGTSKSCSDSFYRNMRWLTQK
SGLYPVQDAQYTNNRGKDILFVWGIHHPPTDTAQTNLYTRTDTTTSVTT
ENLDRTFKPVIGPRPLVNGLIGRINYYWSVLKPGQTLRVRSNGNLIAPW
YGHVLSGESHGRILKTDLNSGNCVVQCQTEKGGLNSTLPFHNISKYAFG
NCPKYIGVKSLKLAIGLRNVPARSSRGLFGAIAGFIEGGWPGLVAGWYG
FQHSNDQGVGMAADRDSTQKAVDKITSKVNNIVDKMNKQYEIIDHEFSE
VETRINMINNKIDDQIQDVWAYNAELLVLLENQKTLDEHDANVNNLYNK
VKRALGSNAMEDGKGCFELYHKCDDQCMETIRNGTYNRRKYKEESRLER
Q

In one aspect, the antigen comprises or consists of the sequence SEQ ID NO: 41; or a variant thereof having at least 80% identity thereto (such as at least 85%, at least 90%, at least 95%, at least 97%, at least 98%, at least 99% identity thereto.

In some aspects, the genetically engineered protein or chemically engineered protein may comprise an ectodomain of the H5N8 gene that lacks both the hemagglutinin gene signal peptide and the transmembrane domain, replaced with a 30 amino acid trimerization foldon sequence of the trimeric protein fibritin from bacteriophage T4.

Suitably, the H5N8 ectodomain for use in the present invention may comprise the amino acid sequence set forth in SEQ ID NO: 80:

(SEQ ID NO: 80)
DQICIGYHANNSTEQVDTIMEKNVTVTHAQDILEKTHNGKLCDLNGVKP
LILKDCSVAGWLLGNPMCDEFIRVPEWSYIVERANPANDLCYPGSLNDY
EELKHLLSRINHFEKILIIPKSSWPNHETSLGVSAACPYQGTPSFFRNV
VWLIKKNDAYPTIKISYNNTNREDLLIMWGIHHSNNAEEQTNLYKNPTT
YISVGTSTLNQRLVPKIATRSQVNGQRGRMDFFWTILKPNDAIHFESNG
NFIAPEYAYKIVKKGDSTIMKSEVEYGHCNTKCQTPVGAINSSMPFHNI
HPLTIGECPKYVKSNKLVLATGLRNSPQGETRGLEGAIAGFIEGGWQGM
VDGWYGYHHSNEQGSGYAADKESTQKAIDGVINKVNSIIDKMNTQFEAV
GREFNNLERRIENLNKKMEDGELDVWTYNAELLVLMENERTLDFHDSNV
KNLYDKVRLQLRDNAKELGNGCFEFYHKCDNECMESVRNGTYDYPQYSE
EARLKREEISGVKLESIGTYQ

In one aspect, the antigen comprises or consists of the sequence SEQ ID NO: 80; or a variant thereof having at least 80% identity thereto (such as at least 85%, at least 90%, at least 95%, at least 97%, at least 98%, at least 99% identity thereto.

With respect to function, the variant should be capable of inducing an immune response. By way of example, the induction of an immune response may be determined by the demonstration of a recall response in peripheral immune cells or splenocytes isolated from a subject previously immunised with the polypeptide or immunogenic fragment thereof. For instance, a recall response may be demonstrated by interferon production (e.g. IFNγ) and/or a proliferative response following an in vitro challenge with an antigen or polypeptide previous used to immunise a subject. (An exemplary assay is provided in Example 1.) Preferably, the variant should be capable of inducing a protective immune response in a subject, against subsequent challenge with avian influenza virus.

### Exemplary Architecture

Examples of engineered protein architecture according to the present invention and for use in vaccines according to the present invention include those listed in Table 6 below; typically the engineered protein will comprise a binding domain as described herein and an antigen as defined herein and may optionally comprise a signal peptide and/or a solubilisation/stabilization or folding domain.

TABLE 6
Exemplary engineered protein architecture

| | | | | | | Solubilisation/ | |
|---|---|---|---|---|---|---|---|
| | | | | | | stabilization/ | |
| | Cell | | Binding | | Signal | folding | |
| Engineered | surface | Binding | domain | | peptide | domain | |
| protein | protein | domain | structure | Antigen | (optional) | (optional) | Subject |
| Genetically | CD83 | Based on | May be | HA | BiP | Foldon | Avian |
| engineered | | antibody | based on an | | | | subject |
| or | | clone | antibody | | | | e.g. |
| chemically | | F890/GE8 | fragment | | | | chicken, |
| engineered | | | such as | | | | turkey |
| | | | scFv, Fv, | | | | duck, |
| | | | F(ab′) or | | | | quail, |
| | | | F(ab′)2 | | | | pigeon |
| | | | | | | | or |
| | | | | | | | goose |
| Genetically | CD11c | Based on | May be | HA | BiP | Foldon | Avian |
| engineered | | antibody | based on an | | | | subject |
| or | | clone 8F2 | antibody | | | | e.g. |
| chemically | | | fragment | | | | chicken, |
| engineered | | | such as | | | | turkey |
| | | | scFv, Fv, | | | | duck, |
| | | | F(ab′) or | | | | quail, |
| | | | F(ab′)2 | | | | pigeon |
| | | | | | | | or |
| | | | | | | | goose |
| Genetically | Dec205 | Based on | May be | HA | BiP | Foldon | Avian |
| or | | antibody | based on an | | | | subject |
| chemically | | clone | antibody | | | | e.g. |
| engineered | | F877/AD6 | fragment | | | | chicken, |
| | | | such as | | | | turkey |
| | | | scFv, Fv, | | | | duck, |
| | | | F(ab′) or | | | | quail, |
| | | | F(ab′)2 | | | | pigeon |
| | | | | | | | or |
| | | | | | | | goose |

An example of domain architecture which may be used in the present invention is, in the following order (SEQ ID NO: 62):

BIP signal-1H9HA ectodomain-Foldon-LINKER-CD83 scFv-V5-His tag

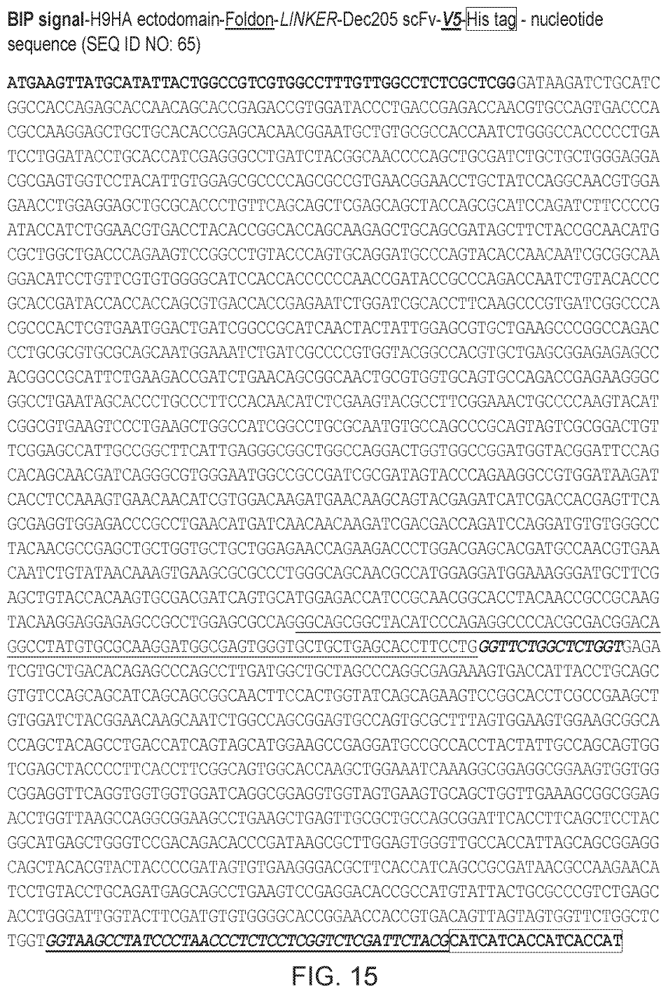

(SEQ ID NO: 62)
MKLCILLAVVAFVGLSLGDKICIGHQSTNSTETVDTLTETNVPVTHAKELLHTEHNGMLCATNLGHPL
ILDTCTIEGLIYGNPSCDLLLGGREWSYIVERPSAVNGTCYPGNVENLEELRTLFSSSSSYQRIQIFP
DTIWNVTYTGTSKSCSDSFYRNMRWLTQKSGLYPVQDAQYTNNRGKDILFVWGIHHPPTDTAQTNLYT
RTDTTTSVTTENLDRTFKPVIGPRPLVNGLIGRINYYWSVLKPGQTLRVRSNGNLIAPWYGHVLSGES
HGRILKTDLNSGNCVVQCQTEKGGLNSTLPFHNISKYAFGNCPKYIGVKSLKLAIGLRNVPARSSRGL
FGAIAGFIEGGWPGLVAGWYGFQHSNDQGVGMAADRDSTQKAVDKITSKVNNIVDKMNKQYEIIDHEF
SEVETRLNMINNKIDDQIQDVWAYNAELLVLLENQKTLDEHDANVNNLYNKVKRALGSNAMEDGKGCF
ELYHKCDDQCMETIRNGTYNRRKYKEESRLERQGSGYIPEAPRDGQAYVRKDGEWVLLSTFLGSGSGD
IVMTQSPSSLAVSVGQKVTMSCTSSQVLLHSPNQKNYLAWYQQKPGQSPKLLVYFASTRESGVPDRFT
GSGSGTDFTLTISSVQAEDLAVYYCQQHYSTPLTFGAGTKLELKGGGGSGGGGSGGGGSGGGGSEVQL
QQSGPELVKPGASVKISCKASGYTFTDYYINWVKQSHGKSLEWIGDINPTNGDSTYSQKFKGKATLTV
DKSSSTAYMELRSLTSEVSAVYYCARDYAMDYWGQGTSVTVSSGKPIPNPLLGLDST [symbol] ;

wherein the H9HA ectodomain is the at least one antigen and 0083 scFv is the at least one binding domain capable of binding to a cell surface protein on an avian antigen presenting cell. The BIP signal domain is a signal peptide, the foldon domain is a domain which improves solubilisation, stabilization and/or folding, V5 and the His tag are used for purifying the engineered protein. It will be appreciated that the BIP signal, foldon and linker domains shown in SEQ ID NO: 42 are optional. The nucleotide sequence, (SEQ ID NO: 65) codon optimised for Dropsohila melanogaster which encodes the amino acid sequence SEQ ID NO: 62 is given in FIG. 15.

An example of domain architecture which may be used in the present invention is, in the following order (SEQ ID NO: 63):

BIP signal-H9HA ectodomain-Foldon-LINKER-CD11c scFv-V5-His tag

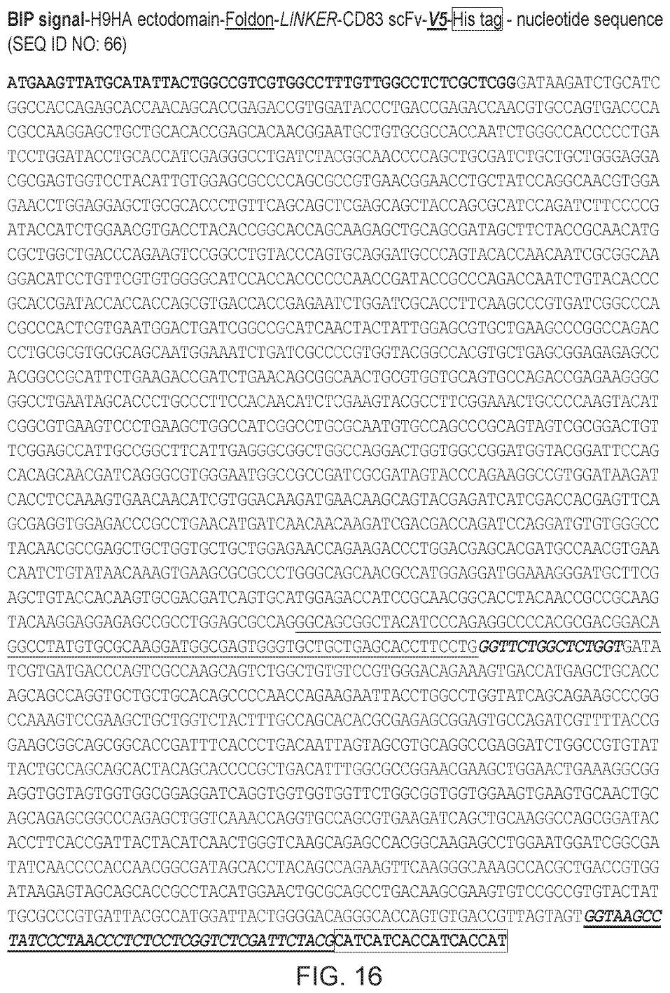

(SEQ ID NO: 63)
MKLCILLAVVAFVGLSLGDKICIGHQSTNSTETVDTLTETNVPVTHAKELLHTEHNGMLCATNLGHPL
ILDTCTIEGLIYGNPSCDLLLGGREWSYIVERPSAVNGTCYPGNVENLEELRTLFSSSSSYQRIQIFP
DTIWNVTYTGTSKSCSDSFYRNMRWLTQKSGLYPVQDAQYTNNRGKDILFVWGIHHPPTDTAQTNLYT
RTDTTTSVTTENLDRTFKPVIGPRPLVNGLIGRINYYWSVLKPGQTLRVRSNGNLIAPWYGHVLSGES
HGRILKTDLNSGNCVVQCQTEKGGLNSTLPFHNISKYAFGNCPKYIGVKSLKLAIGLRNVPARSSRGL
FGAIAGFIEGGWPGLVAGWYGFQHSNDQGVGMAADRDSTQKAVDKITSKVNNIVDKMNKQYEIIDHEF
SEVETRLNMINNKIDDQIQDVWAYNAELLVLLENQKTLDEHDANVNNLYNKVKRALGSNAMEDGKGCE
ELYHKCDDQCMETIRNGTYNRRKYKEESRLERQGSGYIPEAPRDGQAYVRKDGEWVLLSTFLGSGSGE
VQLQQSGPELVKPGASVKMSCKASGYTFTNYVLHWVKQKPGQGLEWIGYINPYNDGTKFNEKFKGKAT
LTSDTSSSTAFMELSSLTSEDSAVYYCARGDNLRPYYFDYWGQGTTLTVSSGGGGSGGGGSGGGGSGG
GGSQIVLTHSPAIMSASPGEKVTMTCSASSSVSFMYWYQQKPGSSPRLLLYDTSSLSSGVPVRFSGSG
SGTSYSLTISRMEAEDAATYYCQQWSRYPPTFGGGTKLEIKGKPIPNPLLGLDST [symbol] ;

wherein the H9HA ectodomain is the at least one antigen and CD11 scFv is the at least one binding domain capable of binding to a cell surface protein on an avian antigen presenting cell. The BIP signal domain is a signal peptide, the foldon domain is a domain which improves solubilisation, stabilization and/or folding, V5 and the His tag are used for purifying the engineered protein. It will be appreciated that the BIP signal, foldon and linker domains shown in SEQ ID NO: 63 are optional. The nucleotide sequence, (SEQ ID NO: 66) codon optimised for Dropsohila melanogaster which encodes the amino acid sequence SEQ ID NO: 63 is given in FIG. 16.

An example of domain architecture which may be used in the present invention is, in the following order (SEQ ID NO: 64):

BIP signal-H9HA ectodomain-Foldon-LINKER-Dec205 scFv-V5-His tag

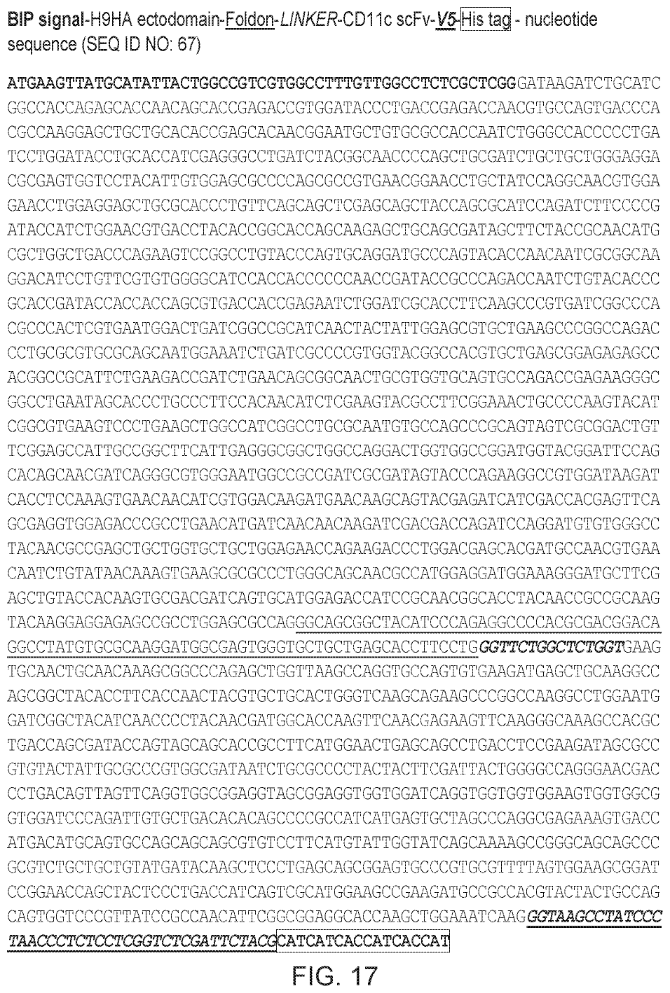

(SEQ ID NO: 64)
MKLCILLAVVAFVGLSLGDKICIGHQSTNSTETVDTLTETNVPVTHAKELLHTEHNGMLCATNLGHPL
ILDTCTIEGLIYGNPSCDLLLGGREWSYIVERPSAVNGTCYPGNVENLEELRTLFSSSSSYQRIQIFP
DTIWNVTYTGTSKSCSDSFYRNMRWLTQKSGLYPVQDAQYTNNRGKDILFVWGIHHPPTDTAQTNLYT
RTDTTTSVTTENLDRTFKPVIGPRPLVNGLIGRINYYWSVLKPGQTLRVRSNGNLIAPWYGHVLSGES
HGRILKTDLNSGNCVVQCQTEKGGLNSTLPFHNISKYAFGNCPKYIGVKSLKLAIGLRNVPARSSRGL
FGAIAGFIEGGWPGLVAGWYGFQHSNDQGVGMAADRDSTQKAVDKITSKVNNIVDKMNKQYEIIDHEF
SEVETRLNMINNKIDDQIQDVWAYNAELLVLLENQKTLDEHDANVNNLYNKVKRALGSNAMEDGKGCE
ELYHKCDDQCMETIRNGTYNRRKYKEESRLERQGSGYIPEAPRDGQAYVRKDGEWVLLSTFLGSGSGE
IVLTQSPALMAASPGEKVTITCSVSSSISSGNFHWYQQKSGTSPKLWIYGTSNLASGVPVRFSGSGSG
TSYSLTISSMEAEDAATYYCQQWSSYPFTFGSGTKLEIKGGGGSGGGGSGGGGSGGGGSEVQLVESGG
DLVKPGGSLKLSCAASGFTFSSYGMSWVRQTPDKRLEWVATISSGGSYTYYPDSVKGRFTISRDNAKN
ILYLQMSSLKSEDTAMYYCARLSTWDWYFDVWGTGTTVTVSSGKPIPNPLLGLDST [symbol] ;

wherein the H9HA ectodomain is the at least one antigen and Dec205 scFv is the at least one binding domain capable of binding to a cell surface protein on an avian antigen presenting cell. The BIP signal domain is a signal peptide, the foldon domain is a domain which improves solubilisation, stabilization and/or folding, V5 and the His tag are used for purifying the engineered protein. It will be appreciated that the BIP signal, foldon and linker domains shown in SEQ ID NO: 44 are optional. The nucleotide sequence, (SEQ ID NO: 67) codon optimised for Drosophila melanogaster which encodes the amino acid sequence SEQ ID NO: 64 is given in FIG. 17.

In one embodiment, the present invention provides a genetically engineered protein comprising: at least one binding domain which is capable of binding to a cell surface protein on an avian antigen presenting cell; and at least one binding domain which is capable of binding to at least one antigenic polypeptide.

An example of domain architecture which may be used in the present invention is, in the following order (SEQ ID NO: 68):

CD33 SIGNAL-IG10 scFv-(Glycine₄Serine)₄linker-CD83 scFv-Ctag

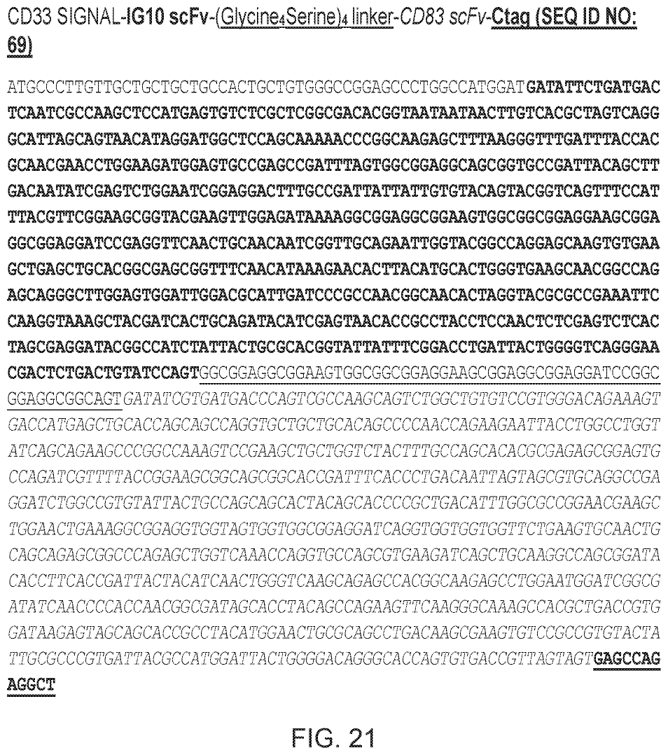

(SEQ ID NO: 68)
MPLLLLLPLLWAGALAMDILMTQSPSSMSVSLGDTVIITCHASQGISSNIGWLQQKPGKSEKGLIYHA
TNLEDGVPSRFSGGGSGADYSLTISSLESEDFADYYCVQYGQFPFTFGSGTKLEIKGGGGSGGGGSGG
GGSEVQLQQSVAELVRPGASVKLSCTASGFNIKNTYMHWVKQRPEQGLEWIGRIDPANGNTRYAPKFQ
GKATITADTSSNTAYLQLSSLTSEDTAIYYCARYYFGPDYWGQGTTLTVSSGGGGSGGGGSGGGGSGG
GGSDIVMTQSPSSLAVSVGQKVTMSCTSSQVLLHSPNQKNYLAWYQQKPGQSPKLLVYFASTRESGVP
DRFTGSGSGTDFTLTISSVQAEDLAVYYCQQHYSTPLTFGAGTKLELKGGGGSGGGGSGGGGSEVQLQ
QSGPELVKPGASVKISCKASGYTFTDYYINWVKQSHGKSLEWIGDINPTNGDSTYSQKFKGKATLTVD
KSSSTAYMELRSLTSEVSAVYYCARDYAMDYWGQGTSVTVSSEPEA;

wherein the IG10 scFv is the at least one binding domain which is capable of binding to at least one antigenic polypeptide and CD83 scFv is the at least one binding domain capable of binding to a cell surface protein on an avian antigen presenting cell. The CD33 signal domain is a signal peptide, Glycine₄Serine₄ is a linker and the C tag is used for purifying the engineered protein. It will be appreciated that the signal, linker and purification tag domains shown in SEQ ID NO: 68 are optional. The nucleotide sequence, (SEQ ID NO: 69) which encodes the amino acid sequence SEQ ID NO: 68 is given in FIG. 21.

Further illustrative embodiments are shown as SEQ ID NO: 72 (nucleotide sequence) and SEQ ID NO: 76 (amino acid sequence). The construct of the invention may comprise or consist of any of the above mentioned sequences, or a variant with at least 80%, 85%, 90%, 95% or 99% sequence identity thereto. The construct of the invention may comprise or consist of SEQ ID NO: 72 or 76, or a variant with at least 80%, 85%, 90%, 95% or 99% sequence identity thereto.

### Nucleic Acids/Nucleic Acid Constructs

As used herein, the terms “polynucleotide” and “nucleic acid” are intended to be synonymous with each other. The nucleic acid sequence may be any suitable type of nucleotide sequence, such as a synthetic RNA/DNA sequence, a cDNA sequence or a partial genomic DNA sequence.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

The present invention provides a polynucleotide which encodes an engineered protein according to the present invention. The present invention provides a polynucleotide which encodes an engineered protein comprising: at least one binding domain which is capable of binding to a cell surface protein on an avian antigen presenting cell; and at least one antigen.

Nucleic acids encoding an engineered protein according to the present invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The polynucleotide may be in isolated or recombinant form. It may be incorporated into a vector and the vector may be incorporated into a host cell. Such vectors and suitable hosts form yet further aspects of the present invention.

The polynucleotide which encodes the engineered protein according to the present invention may be codon optimised. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. Suitably, the polynucleotide may be codon optimised for expression in a specific avian subject.

In one embodiment, there is provided a nucleic acid construct which comprises a first polynucleotide which encodes at least one binding domain which is capable of binding to a cell surface protein on an avian antigen presenting cell as defined herein; and a second polynucleotide which encodes at least one antigenic polypeptide as defined herein.

In one embodiment, the first and second polynucleotides are operably linked to the same promoter. Suitably, a nucleic acid construct according the present invention may encode a genetically engineered protein such as a fusion protein, wherein the first and second polynucleotides are operably linked to the same promoter In some aspects, the nucleic acid construct encodes at least two, at least three, at least four at least five antigens. In this way, a single vaccine will express antigens derived from multiple antigenically divergent viruses and afford protection against multiple pathogens and/or antigenically divergent variants within virus subtypes.

In some aspects, the nucleic acid construct comprises a domain which allows purification of the engineered protein such as an affinity tag. Numerous tags suitable for protein purification are known in the art and include, for example, His tag, polyHis tag, polycysteine tag, FLAG epitope, histidine affinity tag, bacteriophage T7 epitope and other epitope tags.

### Vector

The present invention also provides a vector comprising at least one nucleic acid construct according to the present invention.

The term “vector” as used herein includes an expression vector, i.e., a construct enabling expression of an engineered protein according to the present invention.

Suitably the expression vector enables expression of an engineered protein according to the present invention.

An “expression cassette” comprises a gene of interest (open reading frame (ORF)) and one or more regulatory sequences enabling expression of the gene of interest. Typically expression cassettes comprise a promoter, a gene of interest and a terminator.

In some embodiments, the vector is a multivalent vector. The multivalent vector comprises multiple expression cassettes enabling expression of more than one engineered protein according to the present invention.

In some embodiments, the vector is a cloning vector.

Suitable vectors may include, but are not limited to, plasmids, viral vectors, transposons, nucleic acid complexed with polypeptide or immobilised onto a solid phase particle.

Viral delivery systems include but are not limited to adenovirus vector, an adeno-associated viral (AAV) vector, a herpes viral vector (such as turkey herpes virus (HTV/HVT), retroviral vector, lentiviral vector, baculoviral vector).

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

A detailed list of retroviruses may be found in Coffin et al., (“Retroviruses” 1997 Cold Spring Harbour Laboratory Press Eds: J M Coffin, S M Hughes, H E Varmus pp 758-763), incorporated herein by reference.

Lentiviruses also belong to the retrovirus family, but they can infect both dividing and non-dividing cells (Lewis et al., (1992) EMBO J. 3053-3058), incorporated herein by reference.

The vector may be capable of transferring a polynucleotide the invention to a cell, for example a host cell as defined herein. The vector should ideally be capable of sustained high-level expression in host cells, so that the VH and/or VL domain are suitably expressed in the host cell.

The vector may be a retroviral vector. The vector may be based on or derivable from the MP71 vector backbone. The vector may lack a full-length or truncated version of the Woodchuck Hepatitis Response Element (WPRE).

Examples of viral vectors which may be particularly suitable as vectors for use according to the present invention include a herpes viral vector (such as turkey herpes virus (HTV/HVT), Newcastle disease virus (NDV), Duck enteritis virus (DEV), Avian infectious laryngotracheitis (ILT), Fowl Adenovirus, Marek disease virus (MDV) and Infectious bursal disease virus (IBDV), Infectious bronchitis virus (IBV).

In particular, examples of vectors which may be used in the present invention are HTV/HVT and NDV.

### Cell

The present invention further provides an engineered cell comprising an engineered protein according to the present invention (such as a genetically engineered protein or a chemically engineered protein according to the present invention). In one aspect, the engineered cell may comprise a nucleic acid construct or vector which encodes an engineered protein according to the present invention. The engineered cell may be any cell which can be used to express and produce an engineered protein according to the present invention. Suitably, the cell may be an avian cell. Suitably, the cell may be from a chick, or chicken, turkey, duck, quail, pigeon or goose. Suitably, the cell may comprise viral vector according to the present invention.

### Vaccine/Pharmaceutical Composition

The present invention also provides a vaccine comprising at least one engineered protein(s) of the invention, nucleic acid construct(s) according to the invention, or vector(s) according to the invention or engineered cell(s) according to the invention.

The term “vaccine” as used herein refers to a preparation which, when administered to a subject, induces or stimulates a protective immune response. A vaccine can render an organism immune to a particular disease, for example avian influenza. The vaccine of the present invention thus induces an immune response in a subject which is protective against subsequent pathogen challenge e.g. viral, bacterial or parasite challenge. A vaccine of the invention may be capable of inducing a cross-protective immune response against a plurality of virus genotypes. In an embodiment a vaccine of the invention of a single genotype may be capable of inducing a cross-protective immune response against a plurality of pathogen serotypes, subtypes and genotypes.

Suitably, the vaccine may be a recombinant subunit vaccine.

The vaccine may comprise a plurality of components such as engineered protein(s) according to the invention, nucleic acid construct(s) according to the invention or vector(s) according to the invention and a pharmaceutically acceptable carrier. The plurality of components may correspond to a plurality of different isolates, for example, different isolates of high or unknown virulence. Such a vaccine may be capable of inducing a cross-protective immune response against a plurality of virus genotypes.

In some embodiments, the vaccine is a monovalent vaccine. Suitably, a monovalent vaccine may immunize the subject against a single antigen. In some embodiments, the vaccine is a multivalent or polyvalent vaccine. Suitably, a multivalent or polyvalent vaccine may immunize the subject against two or more antigens from different strains (serotypes/genotypes) of one pathogen in order to immunize against one disease. In some embodiments, the vaccine is a multi-disease or multi-pathogen vaccine. Suitably a multi-disease or multi-pathogen vaccine may comprise protective antigens from two or more pathogens to immunize against two or more diseases.

In some aspects, the vaccine encodes at least two, at least three, at least four at least five different antigens. The antigens may be from divergent variants within a subtype (e.g. different isolates of avian influenza virus) or viruses or may be from divergent viruses (e.g. from avian influenza virus, Newcastle Disease virus, infectious bursal disease virus and infectious bronchitis virus). In this way, a single vaccine may express antigens derived from multiple antigenically divergent pathogens, viruses and/or bacteria and afford protection against multiple pathogens and/or antigenically divergent variants within virus subtypes.

The vaccine may be useful in preventing disease(s). Accordingly, the invention provides a vaccine of the invention for use in treating and/or preventing disease(s).

The present invention also provides a pharmaceutical composition which comprises at least one engineered protein of the invention, nucleic acid construct according to the invention or vector according to the invention. The pharmaceutical composition may be used for treating or preventing disease in a subject.

The vaccine or pharmaceutical composition may optionally comprise one or more adjuvants, excipients, carriers and diluents. The choice of pharmaceutical excipient, carrier or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents. The pharmaceutical compositions typically should be sterile and stable under the conditions of manufacture and storage. Formulations for parenteral administration include, but are not limited to, suspensions, solutions, emulsions in oily or aqueous vehicles, pastes, nanoparticles and implantable sustained-release or biodegradable formulations. Sterile injectable formulations may be prepared using a non-toxic parenterally acceptable diluent or solvent. A pharmaceutical composition of the present invention may include pharmaceutically acceptable dispersing agents, wetting agents, suspending agents, isotonic agents, coatings, antibacterial and antifungal agents, carriers, excipients, salts, or stabilizers which are nontoxic to the subjects at the dosages and concentrations employed. Preferably, such a composition can further comprise a pharmaceutically acceptable carrier or excipient for use in the treatment of disease that is compatible with a given method and/or site of administration, for instance for parenteral (e.g. sub-cutaneous, intradermal, or intravenous injection) administration.

The vaccine or pharmaceutical composition may comprise one or more engineered protein(s) of the invention, nucleic acid construct(s) according to the invention or vector(s) according to the invention in an effective amount.

In an embodiment the invention provides engineered protein(s) of the invention, nucleic acid construct(s) according to the invention or vector(s) according to the invention which when administered to a subject induces an immune response which is protective against subsequent challenge with pathogens. In an embodiment the invention provides engineered protein(s) of the invention, nucleic acid construct(s) according to the invention or vector(s) according to the invention which when administered to a subject induces an immune response which is protective against subsequent challenge with a pathogen of a different subtype or genotype to the pathogen of the vaccine.

### Methods of Prevention/Treatment

The present invention also provides a method of preventing and/or treating a disease in a subject by administration to the subject of an effective amount of an engineered protein of the invention, a nucleic acid construct according to the invention, a vector according to the invention or an avian vaccine according to the invention.

The term “preventing” is intended to refer to averting, delaying, impeding or hindering the contraction of disease. For example, by delivering disease-specific antigens to professional antigen presenting cells, the subject's immune system may be enabled to recognise and eliminate infective cells by a humoral or cellular immune response.

The term “treating” is intended to refer to reducing or alleviating at least one symptom of an existing disease or infection.

Suitably, the challenge pathogen load (such as viral load, bacterial load or parasitic load) in the subject may be decreased by administration to the subject of an effective amount of an engineered protein of the invention, a nucleic acid construct according to the invention, a vector according to the invention or an avian vaccine according to the invention.

Suitably, administration to the subject of an effective amount of an engineered protein of the invention, a nucleic acid construct according to the invention, a vector according to the invention or an avian vaccine according to the invention may elicit production of cross-reactive antibodies.

A vaccine according to the invention may elicit production of antibodies which are capable of targeting two or more antigenically variant antigenic polypeptides from different strains of pathogen e.g. a virus. For example, a vaccine designed to target H9 influenza viruses may provide protection against antigenically variant H9 variants.

Suitably, administration of the vaccine according to the invention elicits a humoral and/or cellular immune response in the subject. Suitably, administration of the vaccine induces an increased humoral and/or cellular immune response relative to the administration of corresponding control. For example, a suitable control may be identical to the vaccine according to the invention except it lacks the at least one binding domain which is capable of binding to a cell surface protein on an avian antigen presenting cell. In other words, the corresponding control may essentially consist of antigen.

Suitably, the administration of the vaccine according to the invention may elicit a faster humoral and/or cellular immune response in the subject compared with a corresponding control. For example, the vaccine according to the invention may elicit a humoral and/or cellular response within 6 days post primary vaccination (ppv).

Typically, the humoral immune response is mediated by antibody molecules that are secreted by plasma cells of vaccinated or infected host immune cells such as B cells. These antibody molecules bind to the pathogen (viruses) and neutralize their ability to infect and cause disease (for example, by blocking the ability of the virus to bind to host cells or enter into host cells).

Suitably, humoral immune response may be measured by determining the titre of antibodies produced in response to immunization and comparing this to a suitable control. For example, a vaccine according to the invention may elicit a higher humoral immune response when compared with a suitable control. Suitably, antigen specific antibodies in sera of immunized subjects may be measured by ELISA. (Example 1 describes the measurement of antigen specific immunoglobulins (Ig) IgY (mammalian IgG equivalent), IgA and IgM antibody levels in the sera were determined by ELISA assay.) In the case of viruses which have the hemagglutinin glycoprotein on their surface, a hemagglutination inhibition assay (HI) may be used to quantify the relative concentration of antibodies.

Typically, cellular immune responses or cell-mediated immunity refers to the activation of host immune cells such as T-cells, macrophages and natural killer cells and the secretion of cytokines that directly or indirectly destroy or block invading pathogens and protect the host from infection.

The production of cytokines and chemokines is an integral part of the cellular immune response. Suitably, the cellular immune response may be determined by measuring the production of inflammatory cytokines such as interferon gamma (IFNγ), interleukin 6 (IL6), interleukin 1 β (IL1β), CXCLi2 (IL8) and interleukin 4 (IL4).

Suitably, the cellular immune response (for example measured by IFNγ, IL6, IL1β, CXCLi2 (IL8) and/or IL4) may be increased by at least 2 fold, at least 5 fold, at least 10 fold, at least 20 fold, at least 30 fold, at least 50 fold, at least 75 fold, at least 100 fold relative to a corresponding control.

The “challenge pathogen load” refers to the value given to the quantity of challenge pathogen (for example, a viral pathogen, bacterial pathogen or parasitic pathogen) in a given volume of fluid.

The “challenge pathogen” refers to the pathogen which the vaccine is directed to.

The “viral load” refers to the value given to the quantity of virus in a given volume of fluid. A higher viral burden usually correlates with the severity of an active viral infection.

The challenge pathogen load in subjects may be determined by performing a plaque assay or quantification of amount of viral RNA using quantitative polymerase chain reaction (qPCR).

### Subject

In one embodiment, the subject may be any avian subject. The subject may be poultry. The subject may be selected from a chicken, turkey, duck, quail, pigeon or goose.

Suitably, the subject vaccinated according to the present invention may be domestic poultry. The subject may be a domestic chicken, turkey, duck, quail, pigeon or goose.

In one embodiment the subject is a chicken and the disease is avian influenza (caused by avian influenza virus).

### Disease

The vaccine of the invention may treat and/or prevent disease in a subject.

The vaccine of the invention may treat and/or prevent disease caused by any pathogen e.g. from viruses, bacteria or parasite.

In some embodiments, the vaccine may treat and/or prevent one or more viral diseases. In some embodiments, the vaccine may treat and/or prevent one or more bacterial diseases. In some embodiments, the vaccine may treat and/or prevent one or more viral diseases and one or more bacterial diseases.

The viral disease may be caused by viruses from the Orthomyxoviridae family. For example, the viral disease may be AIV.

The viral disease may be caused by viruses from the Paramyxoviridae family. For example, the viral disease may be NDV. For example, the viral disease may be caused by aMPV.

The viral disease may be caused by viruses from the Coronaviridae family. For example, the viral disease may be Infectious bronchitis caused by IBV.

The viral disease may be caused by viruses from the Birnaviridae family. For example, the viral disease may be IBD, caused by IBDV.

The viral disease may be caused by viruses from the Anelloviridae family. For example, the viral disease may be Chicken anaemia, caused by CAV.

The viral disease may be caused by viruses from the Reoviridae family. For example, the viral disease may be caused by ARV.

The viral disease may be caused by viruses from the Adenoviridae family. For example, the viral disease may be Egg drop syndrome '76, caused by Duck Atadenovirus A. The viral disease may be caused by Fowl adenoviruses (FAdV's 2, 4, 8, 11) In one embodiment, the vaccine of the invention treats and/or prevents avian influenza.

### Administration

The vaccine of the invention may be administered by any convenient route, such as by injection e.g. intramuscular or subcutaneous. Other suitable routes of administration include intranasal, topical ocular, oral or transdermal. In one embodiment, oral administration comprises adding the vaccine to animal feed or drinking water. In another embodiment, the vaccine may be added to bait for a wild animal, for example bait suitable for wild aquatic birds or wildfowl which may infect domestic poultry and other bird and animal species.

The dose for immunisation may be around 1 μg to around 100 μg. For example the dose may be around 1 μg to around 90 μg, around 1 μg to around 80 μg, around 1 μg to around 70 μg, around 1 μg to around 60 μg, around 1 μg to around 50 μg, around 1 μg to around 40 μg, around 1 μg to around 35 μg, around 1 μg to around 30 μg, around 1 μg to around 25 μg, around 1 μg to around 20 μg, around 1 μg to around 15 μg, around 1 μg to around 10 μg, or around 1 μg to around 5 μg.

The dose for immunisation may be around 2 μg to around 100 μg. For example the dose may be around 2 μg to around 90 μg, around 2 μg to around 80 μg, around 2 μg to around 70 μg, around 2 μg to around 60 μg, around 2 μg to around 50 μg, around 2 μg to around 40 μg, around 2 μg to around 35 μg, around 2 μg to around 30 μg, around 2 μg to around 25 μg, around 2 μg to around 20 μg, around 2 μg to around 15 μg, around 2 μg to around 10 μg, or around 2 μg to around 5 μg. The dose may be around 2 μg to around 35 μg. For example the dose may be around 20 μg to around 35 μg.

The dose may be determined by a veterinary practitioner within the scope of sound veterinary judgment. For example, taking into account dose sparing e.g. the ability to reduce dose without compromising treatment.

The vaccine may be administered following a prime-boost regime. For example, after the first inoculation, the subjects may receive a second boosting administration some time (such as about 6, 7, 14, 21 or 28 days) later. In some aspects, the boosting administration may be at the same dosage as the priming administration. In other aspects, the boosting administration may be at a higher dose than the priming administration.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The publications discussed herein are provided solely for their disclosure prior to the filing date of the present application. Nothing herein is to be construed as an admission that such publications constitute prior art to the claims appended hereto.

### EMBODIMENTS

The following embodiments of the present invention, presented as numbered paragraphs, may be used in combination with the other embodiments described herein:
1. In one embodiment, the present invention provides an engineered protein comprising: at least one binding domain which is capable of binding to a cell surface protein on an antigen presenting cell; and
a) at least one antigenic polypeptide; or
b) at least one binding domain which is capable of binding to at least one antigenic polypeptide.
2. In one embodiment, the present invention provides an engineered protein comprising: at least one binding domain which is capable of binding to a cell surface protein on an avian antigen presenting cell; and
a) at least one antigenic polypeptide; or
b) at least one binding domain which is capable of binding to at least one antigenic polypeptide.
3. An engineered protein according to paragraph 1 or 2, wherein said engineered protein comprises at least one antigenic polypeptide.
4. An engineered protein according to paragraph 1 or 2, wherein said engineered protein comprises at least one binding domain which is capable of binding to at least one antigenic polypeptide.
5. An engineered protein according to any preceding paragraph, wherein said engineered protein is genetically engineered.
6. An engineered protein according to any preceding paragraph, wherein said engineered protein is chemically engineered.
7. An engineered protein according to any of paragraphs 1 to 5, wherein the at least one binding domain which is capable of binding to a cell surface protein on an avian antigen presenting cell; and a) at least one antigenic polypeptide; or b) at least one binding domain which is capable of binding to at least one antigenic polypeptide are comprised in a single recombinant protein.
8. An engineered protein according to any preceding paragraph, wherein the at least one binding domain which is capable of binding to a cell surface protein on an avian antigen presenting cell; and a) at least one antigenic polypeptide; or b) at least one binding domain which is capable of binding to at least one antigenic polypeptide are operably linked.
9. An engineered protein according to any preceding paragraph, wherein the avian antigen presenting cell is at least one of a dendritic cell, macrophage, B cell or natural killer cell.
10. An engineered protein according to any preceding paragraph, wherein the cell surface protein is selected from an immunoglobulin family protein, an integrin family receptor or a C-type lectin.
11. An engineered protein according to any preceding paragraph, wherein the cell surface protein is selected from CD83, CD11c or Dec205.
12. An engineered protein according to any preceding paragraph, wherein the cell surface protein is CD83.
13. An engineered protein according to any of paragraphs 1 to 11, wherein the cell surface protein is CD11c.
14. An engineered protein according to any of paragraphs 1 to 11, wherein the cell surface protein is Dec205.
15. An engineered protein according to any preceding paragraph, wherein the at least one antigen is an avian influenza virus antigen, such as hemagglutinin.
16. An engineered protein according to any of paragraphs 1 to 3 or 5 to 15 wherein the at least one antigenic polypeptide is an avian influenza virus antigenic polypeptide, such as a hemagglutinin antigenic polypeptide.
17. An engineered protein according to any preceding paragraph, further comprising a signal peptide.
18. An engineered protein according to any preceding paragraph, further comprising a domain which improves solubilisation, stabilization and/or folding of the engineered protein.
19. An engineered protein according to any of paragraphs 1 or 4 to 18, wherein the at least one binding domain which is capable of binding to an antigen is capable of binding to an avian antigen. Suitably, the antigen from an avian pathogen may be present on the surface of any pathogen e.g. avian pathogen. For example, the antigen may be present on the surface of a virus, on the surface of a bacterium or on the surface of a parasite. Suitably, the antigen may be present on the surface of an inactivated virus, on the surface of an inactivated bacterium or on the surface of an inactivated parasite.
20. An engineered protein according to any preceding paragraph, wherein at least one binding domain is based on the antigen binding site of an antibody or an antibody fragment such as a single-chain variable fragment (scFv), Fv, F(ab′) or F(ab′)2.
21. A nucleic acid construct which comprises a first polynucleotide which encodes at least one binding domain which is capable of binding to a cell surface protein on an avian antigen presenting cell as defined in any of paragraphs 1 to 20; and a second polynucleotide which encodes at least one antigenic polypeptide or at least one binding domain which is capable of binding to at least one antigenic polypeptide as defined in any of paragraphs 1 to 20.
22. A vector which comprises a nucleic acid construct according to paragraph 21.
23. A vector according to paragraph 22 which is a herpes viral vector (such as turkey herpes virus (HTV/HVT) or Newcastle disease virus (NDV) vector.
24. An engineered cell expressing an engineered protein according to any of paragraphs 1 to 20, or comprising a nucleic acid construct according to paragraph 21, or comprising a vector according to paragraph 22 or 23.
25. An avian vaccine comprising a genetically engineered protein according to any of paragraphs 1 to 20, a nucleic acid construct according to paragraph 21 or a vector according to paragraph 22 or 23 and a pharmaceutically acceptable carrier.
26. An avian vaccine according to paragraph 24 for use in treating and/or preventing disease in a subject.
27. Use of a genetically engineered protein according to any of paragraphs 1 to 20, a nucleic acid construct according to paragraph 21, and/or a vector according to paragraph 22 or 23 in the manufacture of a medicament for the treatment and/or prevention of disease.
28. A method for treating and/or preventing a disease in a subject which comprises the step of administering to a subject an effective amount of a vaccine according to paragraph 25 or 26.
29. A vaccine for use according to paragraph 26, or a method according to paragraph 28, wherein administration of said vaccine elicits a humoral and/or cellular immune response in the subject.
30. A vaccine for use, use of a vaccine or a method according to paragraphs 26 to 29, wherein administration of said vaccine decreases the challenge pathogen load in the subject.
31. A vaccine for use, use of a vaccine or a method according to paragraphs 26 to 30, wherein administration of said vaccine elicits production of cross-reactive antibodies.
32. A vaccine for use, use of a vaccine or a method according to paragraphs 26 to 31, wherein the subject is an avian subject.
33. A vaccine for use, use of a vaccine or a method according to paragraphs 25 to 32, wherein the subject is poultry, for example the subject may be selected from a chicken, turkey, duck, quail, pigeon or goose.
34. A method for the preparation of the vaccine according to paragraph 25, the method comprising the step of admixing a genetically engineered protein according to any of paragraphs 1 to 20, a nucleic acid construct according to paragraph 21, and/or a vector according to paragraph 22 or 23, and a pharmaceutically acceptable carrier.

The invention will now be further described by way of Examples, which are meant to serve to assist one of ordinary skill in the art in carrying out the invention and are not intended in any way to limit the scope of the invention.

### EXAMPLES

### Example 1—Antibody Targeted Vaccines Induce Faster and Stronger Immunity and Protection in Chickens

### Materials and Methods

### Viruses and Cells

A/Chicken/Pakistan/UDL 01/2008 H9N2 virus was propagated in 10-day old specific pathogen free (SPF) embryonated chicken eggs and titrated by plaque assay or TC1D₅₀ on Madin-Darbey canine kidney (MDCK) cells. The virus was inactivated chemically using Beta-propiolactone (BPL) and purified by ultracentrifugation through a continuous 30-60% w/v sucrose gradient.

MDCK cells were maintained in Dulbecco modified Eagle medium (DMEM) supplemented with 10% foetal bovine serum (FBS) and 0.1% penicillin and streptomycin at 37° C., 5% CO₂. Drosophila Schneider 2 (S2) cells were obtained from Invitrogen and maintained in Schneider's insect medium supplemented with 10% FBS at 25° C.

Construction of scFv and H9HA Fused scFv Protein Expressing Plasmids

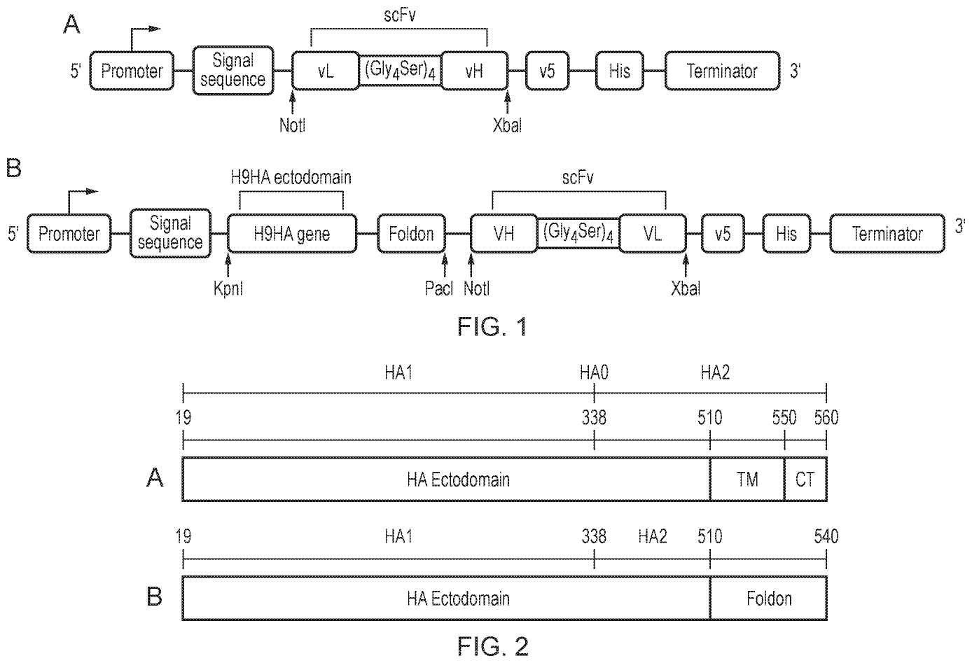

The gene sequences comprising the vL chain and vH chain of chicken Dec205 mAb (clone: IAH F877: AD6), putative chicken CD11c mAb (clone: IAH 8F2) and chicken CD83 scFv mAb (clone: IAH F890:GE8) derived from mouse hybridoma were analyzed commercially (Absolute Antibody Ltd, UK). Synthetic cDNA containing the vL and vH sequences of APC-mAb were joined by (Gly₄Ser)₄ linker peptide sequence and manufactured commercially by Geneart (Thermo Fisher Scientific). The respective individual vL-Linker-vH cDNA were then cloned into Drosophila melanogaster expression vector (pMT/BIP/V5-His version A, Thermo Fisher Scientific) using the Not I and Xba I restriction sites (FIG. 1A). The resultant vector named pMT-BIP-scFv-V5-His was used to insert an ectodomain of the H9HA gene that lacks both hemagglutinin gene signal peptide and the TM domain, replaced with a 30 amino acid trimerization foldon sequence of the trimeric protein fibritin from bacteriophage T4, using Kpn I and Pac I restriction sites (FIG. 1B). The hemagglutinin used in this study was synthetically produced incorporating consensus sequence of hemagglutinin of H9N2 viruses derived from analysis of over 2000 H9HA sequences (from the public database) of G1-like H9 virus lineage using Minimum Sphere Consensus (MScon) method. However, this synthetic hemagglutinin has about 98% amino acid sequence similarity to hemagglutinin ectodomain of A/Chicken/Pakistan/UDL 01/2008 H9N2 virus (GenBank accession number: ACP50708.1, HA1: 19-349 and HA2: 1-174) virus. FIG. 1 shows a schematic representation of an expression cassette and a fusion construct.

Expression and Purification of scFv and H9HA Fused scFv Proteins

Recombinant proteins were produced and purified using the Drosophila Expression System (DES®, Life technologies). Briefly, pMT/BIP/scFv/V5-His or pMT/BIP/H9HA Foldon scFv/V5-His plasmids were co-transfected into Drosophila S2 cells together with a hygromycin B resistance plasmid (pCoHYGRO, Life technologies). Antibiotic selection was carried out for four weeks using hygromycin B at a concentration of 250 μg/ml and single cell clones were obtained via the limiting dilution. Recombinant proteins were secreted into culture supernatant after CuSO₄ (500 μM) induction and then purified by Profinity™IMAC uncharged column (Bio-Rad). Concentration of purified recombinant proteins was determined by Bradford assay and the purity was assessed by SDS-PAGE and Western Blot.

Characterization of scFv and H9HA Foldon-scFv Proteins

Indirect Enzyme Linked Immunosorbent Assay (ELISA) was carried out to examine if Dec205/CD83 scFv and H9HA Foldon-Dec205/CD83 scFv proteins can detect and bind to their respective receptor proteins. The coding sequences of chicken CD83 ectodomain and Dec205 C-type lectin domains 4-5-6 (Staines et al., PLOS One 2013, 8 (1), e51799) were cloned into pMT/BIP/His vector for expression in Drosophila melanogaster S2 cells. Briefly, 8 μg of the respective receptor proteins were added onto the first well of 96 well maxisorp ELISA plates (Thermo Fisher Scientific). Then, two-fold dilutions of the respective receptor proteins were made. The plates were incubated at 4° C. overnight. For detection, the plates were incubated with equimolar concentration of the respective scFv and H9HA Foldon-scFv for two hours at 4° C. This was followed by further incubation with horseradish conjugated (HRP) anti-V5 secondary antibodies. The colorimetric detection was carried out by adding Tetramethylbenzidine (TMB) substrate (Thermo Fisher Scientific) and read at wavelength 450 nm in ELx808 Absorbance Microplate Reader (BioTek).

For the characterization of H9HA Foldon-CD11c scFv, the detection of the corresponding CD11c receptor molecules present in the chicken splenocytes extract was analysed using Western Blot analysis. Briefly, chicken splenocytes were solubilized with 400 μl lysis buffer (NP40) for 30 minutes on ice. During the incubation with the lysis buffer, the lysate was vortexed every 10 minutes. The lysate was then centrifuged at 12,000 rpm for 10 minutes and the resultant supernatant was harvested. The chicken splenocytes extract was run on 10% SDS-PAGE. The protein from the SDS-PAGE gel was blotted onto nitrocellulose membrane and incubated with H9HA Foldon-CD11c scFv overnight at 4° C. This was followed by the incubation with anti-V5 HRP secondary antibody for one hour at room temperature. 3,3′-diaminobenzidine (DAB) substrate (Thermo Fisher Scientific) was then added for detection.

### Flow Cytometry

The binding of the recombinant scFv antibodies to the chicken splenocytes was further investigated using flow cytometry. Briefly, 1×10⁵ chicken splenocytes were stimulated with 200 ng/ml Lipopolysaccharides (LPS, Sigma) for 24 hours. The splenocytes were centrifuged and resuspended in 50 μl of FACS buffer (PBS with 1% Bovine Serum Albumin (BSA) containing 3 μg of respective scFv proteins for 45 minutes at 4° C. After the incubation with scFv proteins, the splenocytes were washed with 150 μl of FACS buffer and resuspended in 100 μl of FACS buffer containing secondary antibody (FITC conjugated anti-V5 tag, 1:100 dilution, Bio-Rad Antibodies) and incubated in the dark for 30 minutes at 4° C. This was followed by fixing of labelled splenocytes with 50 μl of 1% Paraformaldehyde (PFA) for 20 minutes in the dark. The plates were read the next day using MACSQuant flow cytometer and analysed with FCS Express 6 software.

### Bis[Sulfosuccinimidyl] Suberate (BS3) Crosslinking

To determine the oligomeric structure of the recombinant H9HA containing trimerization foldon domain, cross linking was performed using BS3 (Thermofisher Scientific). Briefly, 15 μg recombinant protein was incubated at room temperature in the presence of BS3 (final concentration 10 mM) for one hour. Crosslinking was stopped by the addition of 1M Tris-HCl pH 8.0 to a final concentration of 50 mM. The cross-linked products were separated on SDS gel under reducing conditions, blotted and immunodetected using an anti-H9HA monoclonal antibody.

### Preparation and Stimulation of Chicken Splenocytes

Splenocytes were prepared from the spleens of 3 weeks old unvaccinated Specified Pathogen Free (SPF) chickens via density gradient centrifugation by using histopaque 1083 (Sigma) according to the manufacturer's instructions. About 2×10⁶ cells were plated on each well of 24 well plate suspended in 300 μl of complete Roswell Park Memorial Institute 1640 (RPMI) medium containing 10% FBS and 0.1% penicillin and streptomycin. Cells were treated with 10 μg of H9HA Foldon or 14 μg of H9HA Foldon-scFv (containing 10 μg H9HA Foldon according to the molecular weight) or 10 μg of scFv. Cells were also stimulated with Phorbol Myristate Acetate (PMA)/lonomycin (final concentration 10 μg/ml) as a positive control for IFNγ cytokine production. All cells were stimulated for 5 hours, 22 hours, 30 hours and 45 hours in vitro at 41° C.

RNA Extraction and Quantitative Reverse Transcription PCR (qRT-PCR) of Cytokines and Chemokines

RNA was extracted from the stimulated splenocytes using Rneasy™ kit (Qiagen) according to the manufacturer's protocol. To perform RNA quantification, single-step real time reverse transcription PCR was done using Superscript™ III Platinum One-Step qRT-PCR kit (LifeTechnologies) as per the manufacturer's protocol in 7500 fast real-time PCR machine (Applied Biosystems). Cycling conditions are as follows: i) 5 min hold step at 50° C. ii) a 2 min hold step at 95° C. iii) 40 cycles of 3 sec at 95° C., 30 sec annealing and extension at 60° C. Data were calculated using 2^{-ΔΔCT} approach (n-fold change compared to the media only control group) and reported as values normalized to the expression level of a housekeeping gene RPLPO1. Out of the three reference genes (RPLPO-1, RPL13 and 28S) selected for normalization, RPLPO1 was the most stable gene across the samples hence, chosen for normalization.

### IFNγ Sandwich ELISA

Supernatants from the stimulated splenocytes were harvested and examined by sandwich ELISA. Briefly, anti-chicken IFNγ (1:150 dilution, Invitrogen) was coated onto 96-well maxisorp ELISA plates (Thermo Fisher Scientific). The coated plates were blocked at room temperature with PBS containing 3% BSA for 1 hour. 1:2 dilution of the supernatants was made in PBS buffer containing 3% BSA. The plates were then incubated with the diluted supernatants for two hours at room temperature. Detection was carried out using biotinylated anti-chicken IFNγ detection antibody (1:600 dilution, Invitrogen) for 1 hour at room temperature followed by HRP conjugated streptavidin (1:1000 dilution, Amersham) for another 1 hour at room temperature. 100 μl of Tetramethylbenzidine (TMB) substrate (BD biosciences) was added for 10 minutes. The reaction was stopped using 2M H₂SO₄ and read at wavelength 450 nm in ELx808 Absorbance Microplate Reader (BioTek).

### Hemagglutination Assay (HA) and Hemagglutination Inhibition (HI) Assay

World Health Organisation guidelines were followed for HI assay (World Health Organization. WHO Global Influenza. Surveillance Network. WHO Global Influenza Surveillance Network: Manual for the Laboratory Diagnosis and Virological Surveillance of Influenza. 2011, 153) and HA assay was performed as previously described (Walker, J. M. In Animal Infuenza Virus, 2^{nd} ed.; Spackman, Erica.; Springer Science+Business Media: New York, USA, 2014; pp. 6-10, ISBN 978-1-4939-0757-1). For HI assay 4 HA units of virus was used. Both assays utilized 50 μl of 1% chicken red blood cells (RBCs).

### Chicken Vaccination and Blood Sample Collection

Groups of 7 days old SPF chickens (n=8) were immunized with vaccine dose of containing 2.8 μg, 28 μg and 49 μg of recombinant H9HA Foldon-Dec205 scFv, H9HA Foldon-CD11c scFv or H9HA Foldon-CD83 scFv proteins equivalent to 2 μg, 20 μg and 35 μg of recombinant H9HA Foldon (equimolar concentration). The proteins were formulated in montadine ISA 71 VG (Seppic) adjuvant. The ratio of protein to adjuvant volume was 1:3. The vaccine dose (0.2 ml) was administered subcutaneous, delivered at the back of the neck. Control groups were immunized with PBS or montadine adjuvant. Additionally, one group of chickens (n=8) was vaccinated with inactivated H9N2 virus (A/Chicken/Pakistan/UDL01/2008, 1024 HAU/dose). All vaccinated groups received a booster dose at 14 days old. In all cases, blood samples were collected from the wing vein 6, 14, 21 and 28 days post first vaccination.

### Viral Challenge and Swab Sample Collection

For the viral challenge study, SPF chickens (n=7) were divided into four groups: PBS control, Inactivated H9N2 virus (A/Chicken/Pakistan/UDL 01/2008), H9HA Foldon (25 μg/dose) and H9HA Foldon CD83 scFv (25 μg/dose equivalent of H9HA Foldon). The PBS control and H9HA Foldon-CD83 scFv groups were further divided into two subgroups: Direct virus inoculated and contacts. The chickens were vaccinated at 7 and 14 days old. All chickens except the contact groups were challenged with 1×10⁶ plaque forming units (PFU)/100 μl of A/Chicken/Pakistan/UDL 01/2008 H9N2 virus intranasally at day 22 old (one week after the boost vaccination). Chickens were monitored daily for clinical signs and weight changes throughout the experiment.

Swab samples from buccal and cloacal cavities were collected daily until day 7 post infection with the last sampling performed on day 10 post infection. Sterile polyester tipped swabs were transferred into the virus transport media, vortexed and centrifuged for 10 min at 4500 rpm to clarify the medium. Samples were stored at −80° C. until further analysis.

### Measurement of Serum IgY, IgA and IgM Anti-HA Antibody Levels

Antigen specific IgY (mammalian IgG equivalent), IgA and IgM antibody levels in the sera were determined by ELISA assay. Briefly, flat bottom 96 well maxisorp ELISA plates (Thermo Fisher Scientific) were coated with 1 μg of recombinant H9HA Foldon protein diluted in 50 μL of carbonate buffer (pH 9.6) overnight at 4° C. Protein coated plates were blocked at room temperature with 5% milk powder (Marvel) in PBS-tween 0.1% (PBS-T) for 1 hour. Plates were washed thrice with wash buffer PBS-T. 1:200 dilution of chicken sera was made in PBS-T buffer with 1% milk powder. The plates were then incubated with 50 μl of the diluted sera at room temperature for 1 hour. Plates were again washed thrice and incubated for 1 hour at room temperature with 50 μl of goat anti-chicken IgY, IgA and IgM antibody conjugated to HRP (Abcam) diluted 1:3000 in PBST buffer with 1% milk powder. Plates were washed ×4 with PBS-T then 100 μL of TMB substrate (BD biosciences) was added for 10 minutes. The reaction was stopped using 2M H₂SO₄ and read at wavelength 450 nm in ELx808 Absorbance Microplate Reader (BioTek). A standard serum (serum collected from 35-day old chicken challenged with A/Chicken/Pakistan/UDL 01/2008 (H9N2) virus) was included in all assays. The amount of anti-HA IgY, IgM or IgA antibodies were expressed as sample to reference ratio (relation of absorbance of tested serum sample to absorbance of the reference serum).

### Plaque Assay

The virus titre from the allantoic fluid or swab samples was obtained using plaque assay. Pre-seeded 12 well plates with MDCK cells were inoculated with 10-fold serially diluted samples and left for 1 h at 37° C. Cells were washed with PBS and overlaid with DMEM (1×MEM, 0.21% BSA, 1 mM L-glutamate, 0.15% sodium bicarbonate, 10 mM Hepes, 0.1% penicillin G/streptomycin) containing 0.6% purified agar (Oxoid) and 2 μg/ml TPCK trypsin. Cells were left at 37° C. for 72 hours. After 3 days medium was removed, and cells were stained in crystal violet solution for 30 minutes.

### Virus Microneutralization (MNT) Assay

MDCK cells were pre-seeded into the 96 well plates to reach 90-95% confluency. The immunized chicken sera were inactivated at 56° C. for 30 minutes. Then, 1:200 dilution of the inactivated serum was carried out. This was followed by two-fold serial dilution in triplicates and mixed with 90 μl of A/Chicken/Pakistan/UDL 01/2008 (H9N2) virus containing 150 TCID₅₀. The sera-virus mixture was incubated at 37° C. for 1 hour. Cells were washed with PBS and inoculated with sera-virus mixture for 1 hour at 3TC. After the incubation, cells were washed once again with PBS and serum free DMEM containing 2 μg/ml TPCK trypsin was added, cells were left at 37° C. for 72 hours. After 3 days medium was removed, and cells were stained in crystal violet solution for 30 minutes.

### Statistical Analysis

Results are expressed as the mean±standard deviation (SD). Statistical significance (p-values) was determined using a one-way ANOVA, Log rank (Mantel-Cox) test, unpaired Student's t-test and Tukey's multiple comparison test using Prism™ 8.3.0 (GraphPad Software). Differences were considered statistically significant if P<0.05.

### Results

### Expression and Purification of the Recombinant Proteins

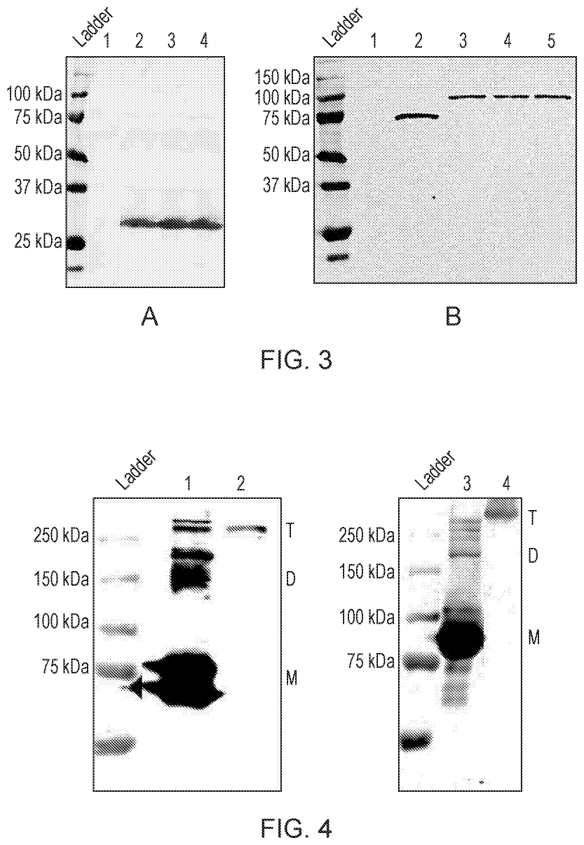

To create a soluble H9HA protein, the TM domain of H9HA was replaced with 30 amino acid long foldon of the trimeric protein fibritin from bacteriophage T4 (FIG. 2). Furthermore, the soluble H9HA protein was recombinantly fused with scFv antibodies targeting Dec205, CD11c and CD83 receptor proteins on chicken APCs. The scFv and H9HA Foldon-scFv proteins were successfully expressed in Drosophila S2 cells. Subsequent purification of the recombinant proteins by His-tag affinity chromatography produced proteins with the expected molecular weights of about 30 kDa for scFv, 70 kDa for H9HA Foldon and 100 kDa for H9HA Foldon-scFv (FIG. 3). Based on recovered purified proteins, it was estimated that expression levels of recombinant proteins range from 10-20 mg/litre of culture supernatants.

### H9HA Ectodomain Fused to T4 Bacteriophage Foldon can Trimerize and Retain the Hemagglutination Activity

The oligomerization state of soluble H9HA protein with trimerization foldon was determined by cross-linking using BS3. Multimeric proteins exposed to this cross-linker will have each subunit crosslinked together with the formation of amide bonds. This provides direct evidence for their close proximity. This also helps to stabilize the structure of oligomers, allowing them to be analysed on SDS denaturing gels for Western blot analysis. This method was used to confirm the native structure of the protein. Recombinant H9HA Foldon and H9HA Foldon-scFv proteins were exposed to BS3 cross linker and the cross-linked products were separated on SDS gel under reducing and denaturing conditions, blotted and immunodetected using anti-H9HA monoclonal antibody. The result is shown in FIG. 4. Without BS3 crosslinking, three species; monomer, dimer and trimer are observed (major band at monomer; Lane 1 and Lane 3 corresponding to about 70 kDa and 100 kDa for H9HA Foldon and H9HA Foldon-scFv respectively). With crosslinking, stable trimeric form is observed (Lane 2 and Lane 4 corresponding to about 210 kDa and 300 kDa for H9HA Foldon and H9HA Foldon-scFv respectively). This indicates that the native structure of recombinant H9HA protein with foldon is a trimer.

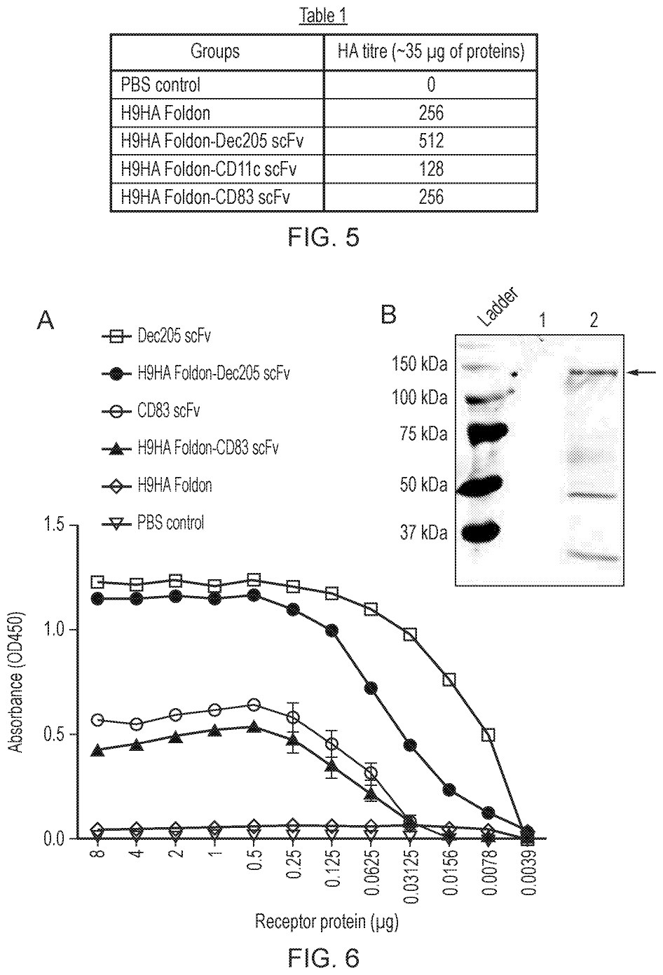

Next, the biological activity of soluble H9HA Foldon and H9HA Foldon scFv proteins was tested using HA assay (FIG. 5). The soluble H9HA Foldon protein, both on its own and when fused to scFv antibodies was able to agglutinate chicken RBC's retaining its hemagglutination activity. Furthermore, on average soluble H9HA Foldon and H9HA Foldon scFv showed hemagglutination upto 0.14 μg/ml. Lower concentrations of soluble H9HA Foldon and H9HA Foldon scFv and PBS control showed no hemagglutination activity. Results are shown in FIG. 5.

The Fusion of scFv Antibodies to H9HA Foldon Protein does not Affect their Function

The specificity of scFv antibodies was demonstrated by binding to chicken splenocytes (FIG. 6A). Chicken splenocytes were stained by all the scFv antibodies. In order to determine if the scFv antibodies can retain their activity after being fused to H9HA Foldon protein, indirect ELISA was carried out. Both Dec205/CD83 scFv and H9HA Foldon fused Dec205/CD83 scFv antibodies were able to detect and bind to their respective receptor proteins and as expected the ability of the scFv antibody to bind to their receptor protein decreased with the fusion to hemagglutinin protein. Since the coding sequence of chicken CD11c receptor protein was publicly unavailable, we were unable to express chicken CD11c receptor protein and use it to characterize H9HA Foldon-CD11c scFv. However, for testing the activity of CD11c scFv fused to H9HA Foldon, we carried out western blot analysis. H9HA Foldon-CD11c scFv was able to detect 150 kDa CD11c protein in the chicken splenocytes extract (FIG. 6B).

Activation of Chicken Splenocytes by scFv, H9HA Foldon and H9HA Foldon-scFv Proteins Ex Vivo

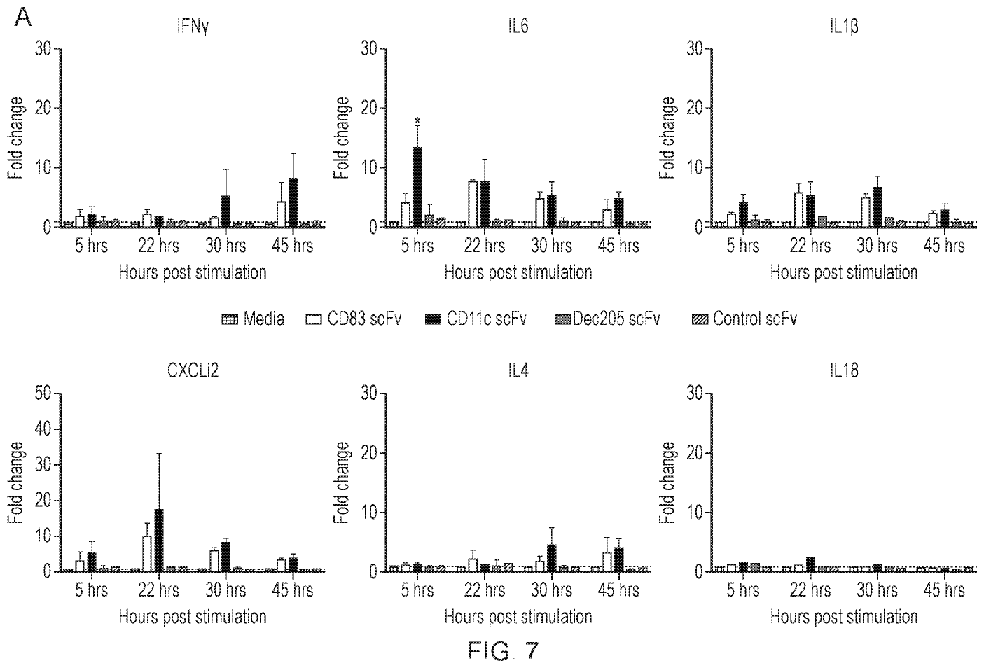

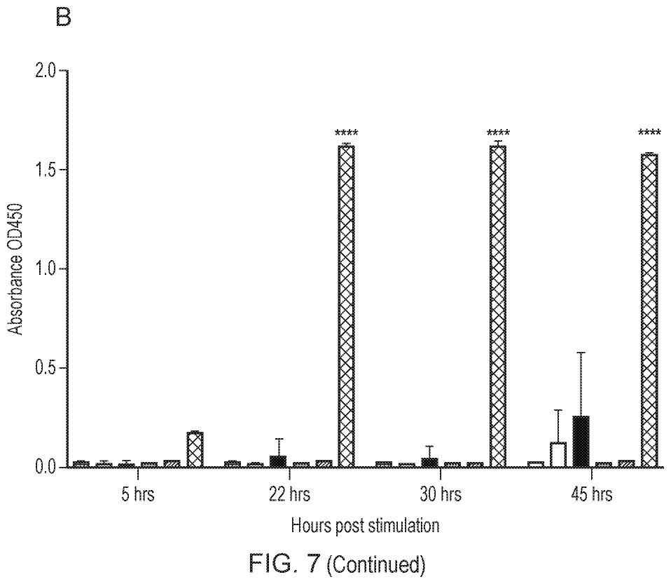

Splenocytes were isolated from unvaccinated SPF chickens and treated with scFv, H9HA Foldon and H9HA Foldon-scFv proteins for 5 hours, 22 hours, 30 hours and 45 hours in vitro. We investigated the production of cytokines IFNγ, IL6, IL1β, IL4, IL18 and chemokine CxCLi2. With regards to stimulation by scFv, CD83 scFv and CD11c scFv were able to induce expression of IL6 (˜5-15 fold, with CD11c scFv ˜13 fold, p<0.05 at 5 hours post stimulation), IL1β (˜5-10 fold) and CxCLi2 (˜5-20 fold) compared to the control scFv (FIG. 7A). There was little or no expression of IL4 and IL18 cytokines. There was a low expression of IFNγ at 45 hours post stimulation by CD11c scFv and CD83 scFv. This was verified by IFNγ ELISA (FIG. 7B).

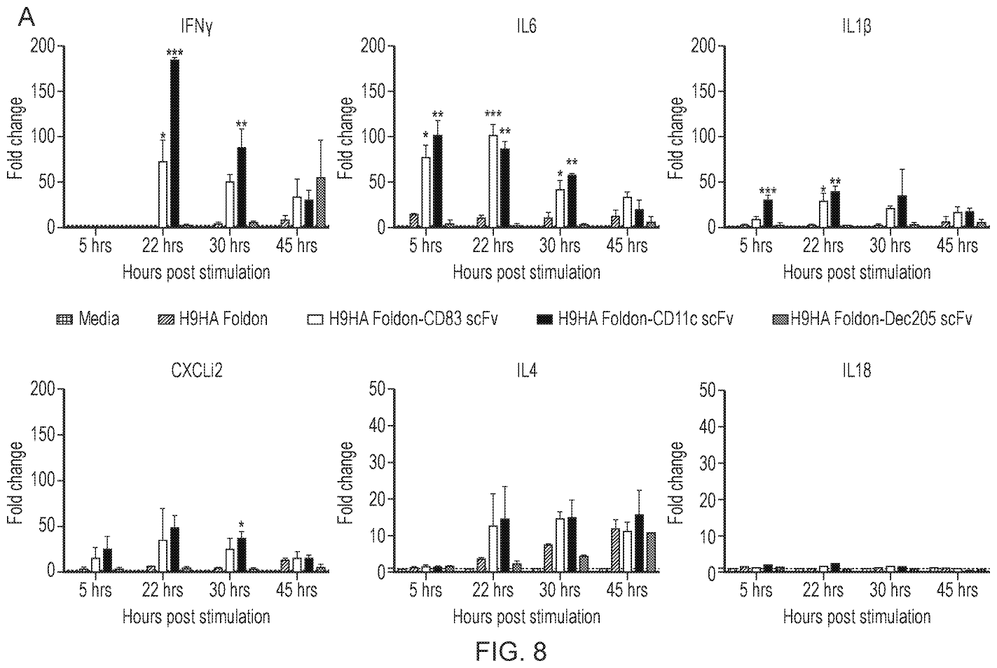

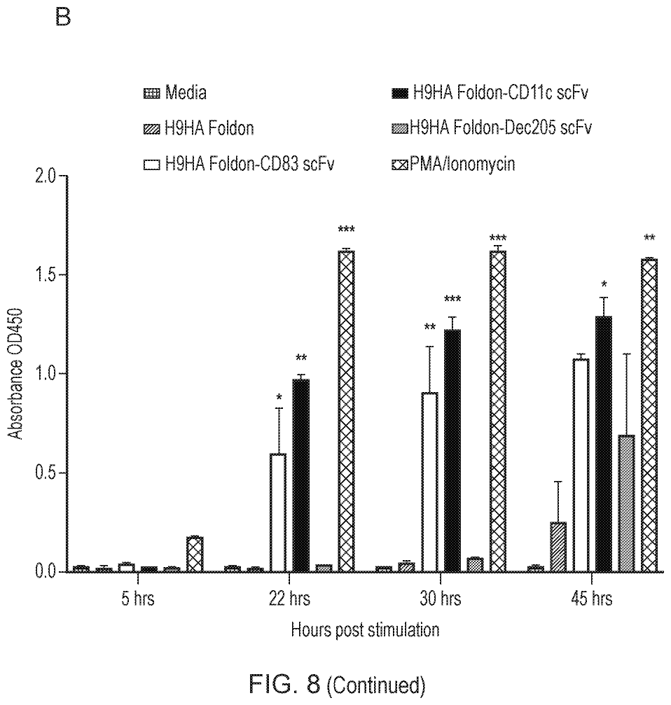

Interestingly, H9HA Foldon-CD83 scFv and H9HA Foldon-CD11c scFv were able to induce significantly higher levels of pro-inflammatory cytokines IFNγ (˜50-180 fold), IL6 (˜50-115 fold) and IL1β (˜30-45 fold) compared to H9HA Foldon (FIGS. 8A and 8B). IL6 and L1P cytokines were induced earlier (5 hours post stimulation) whereas IFNγ was induced later (after 22 hours post stimulation) by H9HA Foldon-CD83 scFv and H9HA Foldon-CD11c scFv. There was no expression of IL18 cytokine. In addition, H9HA Foldon-CD83 scFv and H9HA Foldon-CD11c scFv induced higher levels of IL4 (˜15 fold) and chemokine CxCLi2 (˜15-55 fold) at 22 hours and 30 hours post stimulation (not significant) compared to H9HA Foldon.

Immunization with H9HA Foldon-scFv Proteins Induces Faster and Higher Humoral Response

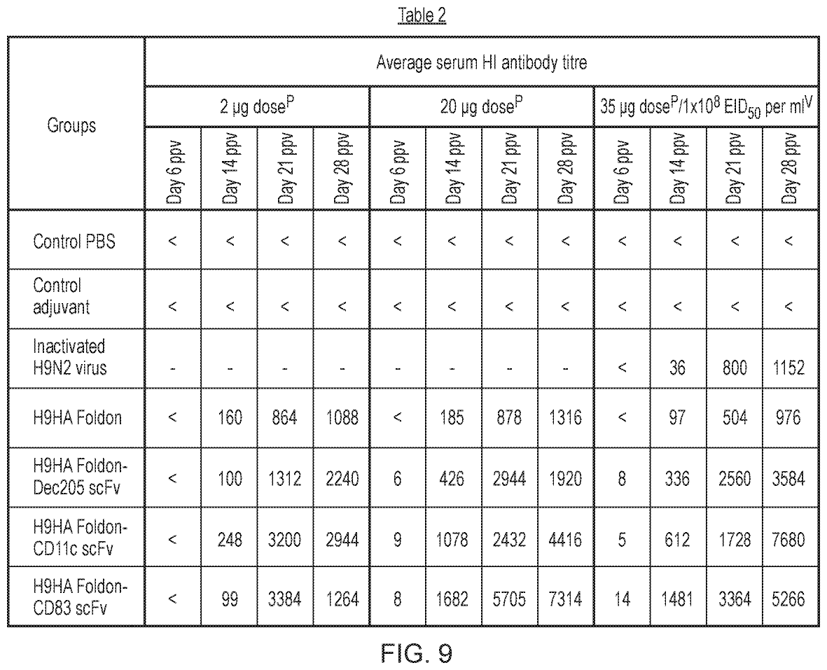

A standard HI assay was conducted to test the ability of H9HA Foldon-scFv to generate HI antibodies. HI antibody titres were measured at 6, 14, 21 and 28 days post primary vaccination. With 20 μg and 35 μg dose of H9HA-scFv we were able to detect HI antibodies as early as day 6 post primary vaccination (ppv) (FIG. 9, Table 2). 2 μg dose of H9HA-scFv was found insufficient to induce earlier antibody response like that induced by 20 μg and 35 μg doses. However, after 21 days ppv even the 2 μg H9HA Foldon-scFv vaccine group had HI antibody titre either higher or similar to that of inactivated vaccine group. Furthermore, with 20 μg and 35 μg doses of H9HA Foldon-CD83 scFv and H9HA Foldon-CD11c scFv, the titre of HI antibodies produced was significantly higher on all days tested compared to the H9HA Foldon. However, with H9HA-Dec205 scFv significantly higher HI titre was produced only with 35 μg dose compared to H9HA Foldon on most of the days tested. Interestingly, there were no significant differences between the three doses of vaccination in H9HA Foldon immunized groups on all the days tested. However, with H9HA Foldon-scFv higher HI antibodies were produced with 20 μg and 35 μg doses compared to 2 μg dose on most of the days tested. Furthermore, the HI antibody titre with 20 μg and 35 μg of H9HA Foldon-scFv was also higher than that of the inactivated virus vaccine group.

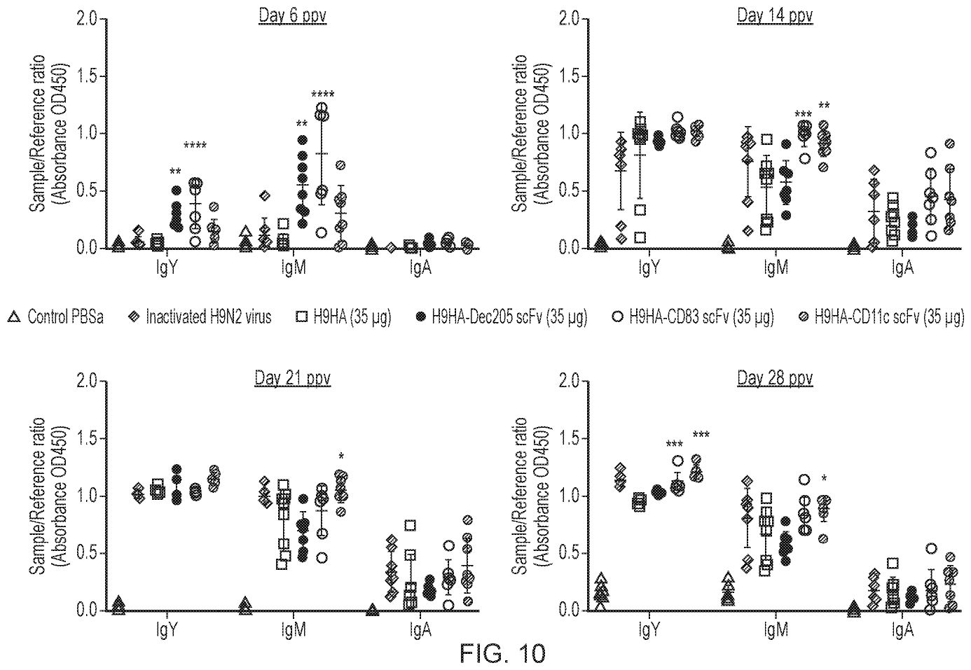

The HI assay only takes account of the antibodies that can block influenza hemagglutinin glycoprotein binding to sialic acid residues of receptor proteins and prevent hemagglutination of RBCs. However, it misses all other antibodies that could possibly neutralize the virus via different route and hence, does not give the total measure of anti-HA antibodies produced in the immunized serum. Thus, we used ELISA to measure the total amount of anti-HA IgY, IgM and IgA antibodies in the serum of the immunized chickens with 35 μg dose of vaccines at 6, 14, 21 and 28 days ppv. As expected, the amount of IgY and IgM antibodies were higher than that of IgA antibodies in the immunized serum (FIG. 10). On day 6 ppv the amount of IgY and IgM antibodies were significantly higher in H9HA Foldon-CD83 scFv and H9HA Foldon-Dec205 scFv groups compared to H9HA Foldon group. Furthermore, there was no difference in the amount of IgY antibodies between any groups on day 14 and day 21 ppv, but a significantly higher amount of IgM antibodies was seen with H9HA Foldon-CD83 scFv and H9HA Foldon-CD11c scFv compared to H9HA Foldon. On day 28 ppv a significantly higher amount of IgY and IgM antibodies were produced with H9HA Foldon-CD11c scFv, whereas with H9HA Foldon-CD83 scFv produced significantly higher anti-HA IgY compared to the H9HA Foldon group. Overall, differences were observed between H9HA Foldon and H9HA Foldon-scFv groups only with anti-HA IgM antibodies.

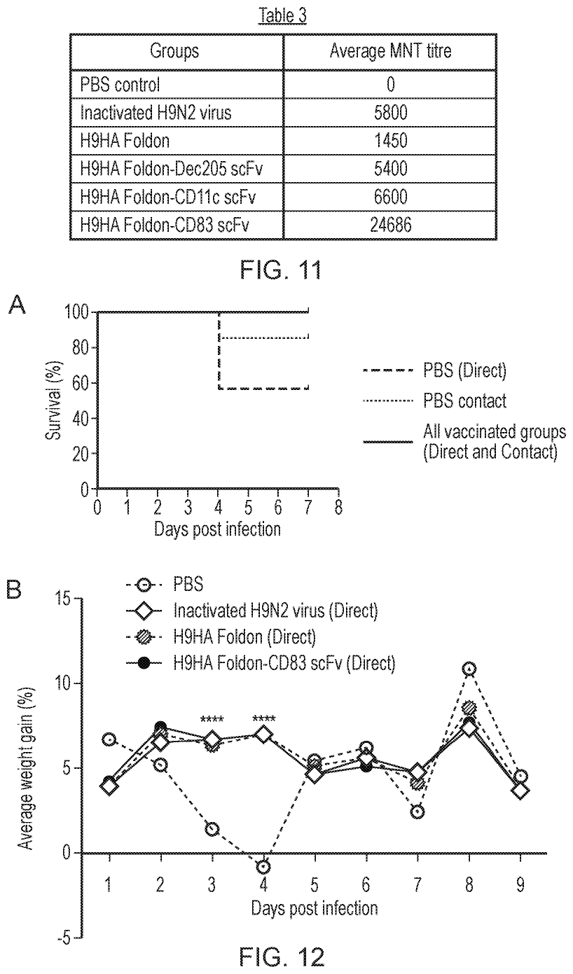

In addition, virus MNT assay was also performed with 35 μg immunized sera at day 28 ppv. The virus neutralization titre with all H9HA Foldon-scFv groups were significantly higher than H9HA Foldon group, and H9HA Foldon-CD83 scFv gave the highest titre compared to all other vaccinated groups (FIG. 11, Table 3).

H9HA Foldon-CD83 scFv is Better at Reducing the Viral Load in H9N2 Virus Challenged Chickens Compared to H9HA Foldon

To determine the protective efficacy of H9HA Foldon-CD83 scFv against the H9N2 infection, different groups of chickens were vaccinated twice with H9HA Foldon, H9HA Foldon-CD83 scFv and inactivated H9N2 vaccines, and challenged with H9N2 virus after 7 days post boost vaccination. The control (PBS treated) and H9HA Foldon-CD83 scFv vaccinated groups also had un-infected chickens serving as contacts. The contact groups were there to provide evidence on whether the vaccinated chickens have a reduced chance of getting infection from the unvaccinated directly infected chickens while sharing the same air space, food and water.

The clinical signs observed in the virus infected chickens include diarrhoea, rapid breathing, weight loss, half eyes shut, ruffled feathers and isolated behaviour. All vaccinated groups (direct and contact) had 100% survival rate whereas only about 58% of the chickens in directly infected PBS control group survived the virus challenge. In addition, the survival rate of PBS treated contact group chickens was only about 87% (FIG. 12A). Furthermore, the average weight gain of all directly infected vaccinated chickens remained fairly consistent after the virus infection, whereas there was a significant decrease in the average weight gain in directly infected PBS control group on day 3 and day 4 post virus infection (FIG. 12B). We lost total of 3 chickens in directly infected PBS control group on day 3 and day 4 post virus infection, and the chickens that survived had a similar average weight gain pattern as the vaccinated groups after day 4 post virus infection.

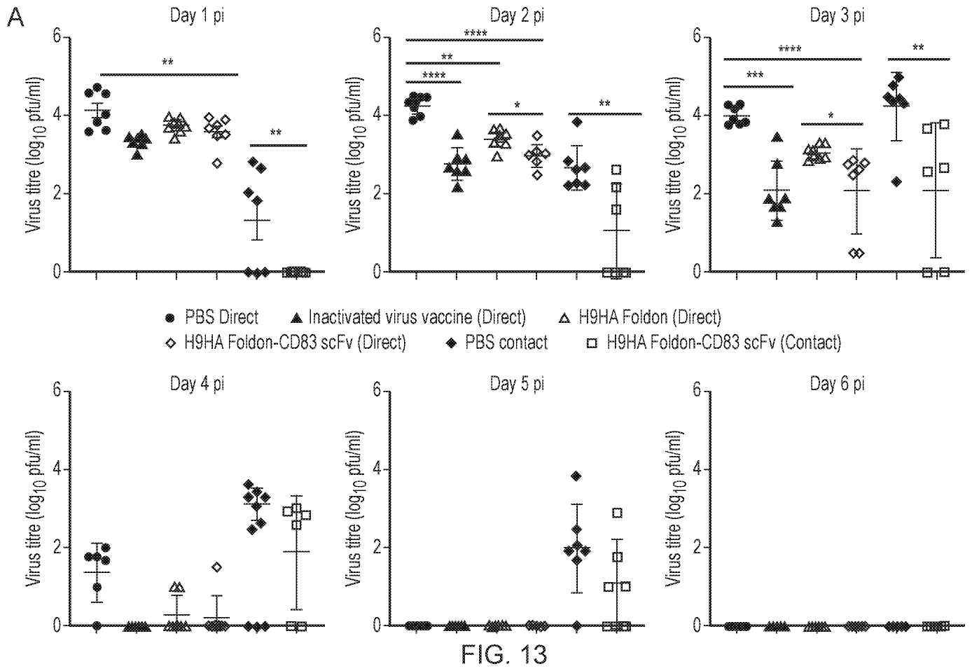

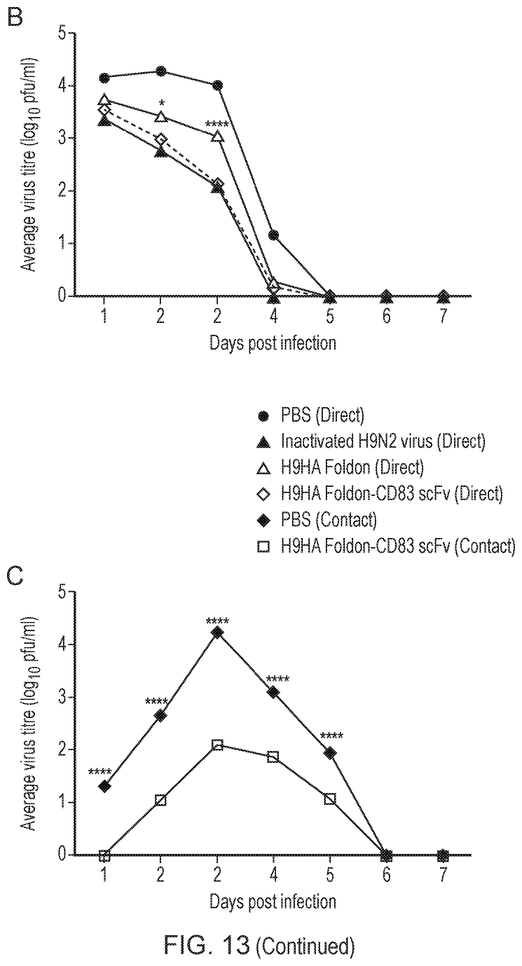

The viral load in chickens was determined by performing plaque assay on the buccal swabs collected on day 1 to day 7 post infection. No virus shedding was observed via the cloacal route (data not shown). On day 1, 2 and 3 post infection significantly lower virus titre was observed in the vaccinated groups compared to the PBS control group (Figure). The average virus titre in directly infected PBS control groups ranged from 23,000 pfu/ml to 12,000 pfu/ml on the first three days post virus infection, whereas for all vaccinated groups the average virus titre ranged from 6200 pfu/ml to 390 pfu/ml. Furthermore, H9HA Foldon-CD83 scFv vaccinated chickens had significantly lower average virus titre compared to H9HA Foldon vaccinated chickens on day 2 post infection (H9HA Foldon-CD83 scFv: 1220 pfu/ml, H9HA Foldon: 3052 pfu/ml) and day 3 post infection (H9HA Foldon-CD83 scFv: 390 pfu/ml, H9HA Foldon: 1257 pfu/ml). We did not see any significant difference between H9HA Foldon-CD83 scFv and inactivated virus vaccine groups in terms of virus load on all days post virus infection. By day 4 post infection, the virus was almost cleared from all the directly infected vaccinated groups whereas some virus was still left in the directly infected PBS control group. On the other hand, the H9HA Foldon-CD83 scFv contact group chickens had significantly lower virus titre compared to PBS contact group, and the chickens in vaccinated contact group showed virus one day later than PBS contact group (FIG. 13). By day 6 post infection the virus was cleared from all the groups (direct and contacts).

### Summary

This study provides evidence that TADV or ATV containing antigens fused with antibodies specific to receptor molecules on the surface of APCs induce faster and stronger immunity in chickens. The prototype TADV consisted of hemagglutinin antigen of H9N2 AIV as a model antigen that was fused with scFv antibodies specific for chicken APC receptors CD83, CD11c and Dec205. The resultant modified hemagglutinin antigen fused with CD83 scFv, CD11c scFv or Dec205 scFv antibodies were produced as recombinant soluble trimeric glycoproteins in insect cells and characterized using Western blot and ELISA assays. The results suggested that the fusion of hemagglutinin antigen to scFv antibodies does not abrogate the functional activity of hemagglutinin or the scFv antibodies. Immunizations of chickens with these APC-targeted H9HA Foldon-scFv vaccines induced faster and stronger hemagglutinin antigen-specific antibody responses compared to the untargeted counterpart or the conventionally killed H9N2 virus vaccine.

For example, recombinant H9HA Foldon-CD83 scFv induced higher serum HI and virus neutralizing antibodies compared to the untargeted H9HA Foldon. Furthermore, chickens vaccinated with TADV (H9HA Foldon-CD83 scFv) also showed reduced clinical disease signs and reduced shedding of virus from buccal cavities when challenged with H9N2 virus. These studies demonstrated that targeting antigens via antibodies to chicken APCs enhanced the immunogenicity and protective efficacy of poultry vaccine against AIV. In addition, H9HA Foldon-scFv were also able to stimulate chicken splenocytes in vitro inducing the expression of pro-inflammatory cytokines (IFNγ, IL1β and IL6) and chemokine (CXCLi2). Hence, scFv antibodies could be serving as built-in adjuvants along with delivering antigen cargo to the APCs, and increasing the immunostimulatory potential of the antigen.

### Example 2—Antibody Targeted Vaccines Show Enhanced Immunogenicity Compared with Commercial Vaccines

### Viruses and Vaccines

The hemagglutinin used for making the recombinant H9HA Foldon and recombinant H9HA Foldon-CD83 scFv was synthetically produced by incorporating consensus sequence of hemagglutinin of H9N2 viruses derived from analysis of over 2000 H9HA sequences (from the public database) of G1-like H9 virus lineage. This synthetic hemagglutinin has about 98% amino acid sequence similarity to hemagglutinin ectodomain of A/Chicken/Pakistan/UDL 01/2008 (UDL 01/08) H9N2 virus (GenBank accession number: ACP50708.1, HA1: 19-349 and HA2: 1-174) virus.

The commercial vaccine tested was inactivated virus vaccine and had mixtures of A/Chicken/UAE/415/99 (UAE/415) H9N2 virus and Newcastle disease (ND) virus.

### Chicken Vaccination and Blood Sample Collection

This animal study was carried out in order to compare the performance of the recombinant antibody targeted vaccines with the currently available commercial avian influenza vaccine. Chickens were divided into five groups (n=10 per group): commercial vaccine, H9HA Foldon (35 μg per dose), H9HA Foldon-CD83 scFv (equivalent to 35 μg of H9HA Foldon according to the molecular weight), Inactivated H9N2 (A/Chicken/Pakistan/UDL 01/2008, the sequence of which is publicly available at GenBank accession number: ACP50708.1) and unvaccinated control groups. H9HA Foldon, H9HA Foldon-CD83 scFv groups were further divided into single and double vaccination groups where the later received boost vaccination. All vaccines were formulated as per the industrial requirement. The volume of the recombinant vaccines and inactivated H9N2 vaccine were kept at 0.2 ml per dose to keep it consistent with the previous experiments. However, the volume of the commercial vaccine was kept at 0.25 ml per dose as per the industrial requirement. The chickens were vaccinated at 1 day old (SPF White Leghorn) subcutaneously, as this mimic the mass application route of 1-day old chickens in the hatchery. Only H9HA Foldon, H9HA Foldon-CD83 scFv and inactivated H9N2 vaccine groups were given boost vaccination at day 7 old. In all cases, blood samples were collected from the wing vein 6, 14, 21, 28 and 35 days post vaccination.

### Hemagglutination Assay (HA) and Hemagglutination Inhibition (HI) Assay

World Health Organisation guidelines were followed for the HI assay and HA assay. Both assays utilized 1% chicken red blood cells (RBCs).

### Results

### Antigenic Relationship Between the Commercial and Recombinant Vaccine Virus Strains

The H9N2 virus strains used in the recombinant H9HA Foldon/H9HA Foldon-CD83 scFv vaccines the commercial vaccine were A/Chicken/Pakistan/UDL01/2008 and A/Chicken/UAE/415/99 respectively. These two viruses had 94% amino acid sequence similarity. We carried out the HI assay on the antisera using both the homologous and heterologous viruses, in order to determine the antigenic relationships between the two viruses. The ‘r’ value is used to determine the extent of the antigenic difference between the two virus strains as described in Archetti et al., J Exp Med, 1950 Nov. 1; 92(5): 441-62.

| | Virus | |
|---|---|---|
| Vaccine (Antisera) | UAE/415 | UDL January 2008 |
| Commercial vaccine (UAE/415) | 103.97 | 630.35 |
| Inactivated H9N2 Pirbright (UDL | 18.38 | 207.94 |
| January 2008) | | |

Table 5: HI titre of the commercial vaccine and inactivated H9N2 Pirbright vaccine antisera with both homologous and heterologous viruses. The homologous titres are indicated in bold.

The ‘r’ value between the two H9N2 viruses was 0.73 meaning these two viruses were antigenically similar (‘r’ value=1 indicates no antigenic difference). Hence, some cross-reactivity between the antisera is expected.

### Analysis of the Antisera

Hi Antibody Titre with A/Chicken/Pakistan/UDL 01/2008 H9N2 Virus

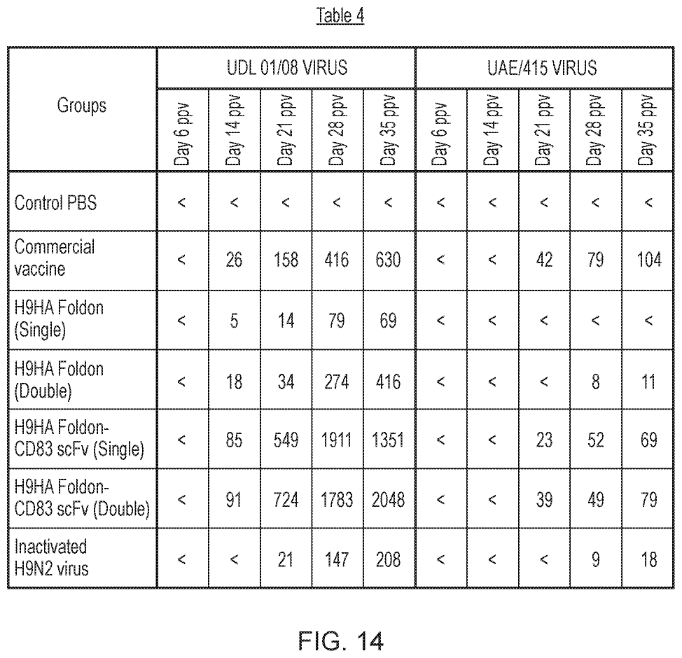

A standard HI assay was conducted to test the antisera from the immunized chickens. A/Chicken/Pakistan/UDL 01/2008 was used for this assay. This virus is homologous for H9HA Foldon, H9HA Foldon-CD83 scFv and Inactivated H9N2 Pirbright vaccines and heterologous for the commercial vaccine. HI antibody titres were measured at 6, 14, 21, 28 and 35 days post primary vaccination (ppv). The results show that the titre of HI antibodies produced by H9HA Foldon-CD83 scFv is significantly higher than H9HA Foldon on all the days tested. Interestingly, there were no significant differences between the single and double vaccinated H9HA Foldon/H9HA Foldon-CD83 scFv groups i.e. the HI antibody titres with and without the boost vaccination were similar. Furthermore, we also saw good cross-reactivity of the commercial vaccine antisera with heterologous UDL 01/08 virus. However, no significant differences were observed between the HI antibody titres between the commercial vaccine and H9HA Foldon-CD83 scFv groups (both single and double vaccinated groups, FIG. 14, Table 4). The HI antibody titre of H9HA Foldon-CD83 scFv group was higher (not significant) than the commercial vaccine group on most of the days tested (Table 5). Moreover, HI antibody production in the immunized chickens were observed after 14 days post primary vaccination.

Hi Antibody Titre with A/Chicken/UAE/415/99

A standard HI assay was conducted to test the antisera from the immunized chickens. A/Chicken/UAE/415/99 was used for this assay. This virus is heterologous for H9HA Foldon, H9HA Foldon-CD83 scFv and Inactivated H9N2 Pirbright vaccines and homologous for the commercial vaccine. HI antibody titres were measured at 6, 14, 21, 28 and 35 days post primary vaccination. As expected, we saw reduction in the HI antibody titre of H9HA Foldon, H9HA Foldon-CD83 scFv and Inactivated H9N2 Pirbright vaccine antisera with heterologous UAE/415 virus. Surprisingly, antisera from chickens immunized with H9HA Foldon-CD83 scFv had higher cross-reactivity with UAE/415 virus compared to H9HA Foldon and Inactivated H9N2 Pirbright vaccine. In addition, HI antibody titre of H9HA Foldon-CD83 scFv group was similar to that of the commercial vaccine on all the days tested (FIG. 14, Table 4). HI antibody production in the immunized chickens were observed after 14 days post primary vaccination.

### Summary

In this study, we evaluated the immunogenicity of H9HA Foldon-CD83 scFv vaccine in comparison with a commercial avian influenza vaccine. There were differences in the vaccine virus strains between H9HA Foldon-CD83 scFv vaccine (A/Chicken/Pakistan/UDL01/2008) and commercial vaccine (A/Chicken/UAE/415/99). However, these viruses have 94% amino acid similarity and ‘r’ value of 0.73 which suggested that these two vaccine virus strains were antigenically similar. With homologous virus UDL 01/08, the HI antibody titre of H9HA Foldon-CD83 scFv group was higher (not significant) than the commercial vaccine group. Interestingly, the HI antibody titre induced by H9HA Foldon-CD83 scFv with the heterologous UAE/415/99 virus was similar to that induced by the commercial vaccine. This suggests that targeting H9HA antigen with CD83 scFv could improve the cross reactivity of the vaccine to other heterologous viruses hence, this could be a strategy to enhance broadly cross-reactive antibody titres. Furthermore, no significant differences were observed in the HI antibody titres between the single and double vaccinated H9HA Foldon/H9HA Foldon-CD83 scFv groups. This provides evidence that a single vaccination schedule without a boost can enhance the immunogenicity of H9HA Foldon-CD83 scFv vaccine. This is very advantageous because a single vaccination is preferred in the field to reduce cost and time.

Overall, these findings suggest that H9HA Foldon-CD83 scFv subunit vaccine may perform better than the commercial whole killed virus vaccine.

### Example 3—Antibody Targeted Vaccine Comprising Bispecific or Multispecific Binding Domains (IG10)

A bispecific single chain fragment variable (scFv) antibody having binding specificity for two antigens was generated. One scFv binds to the virus antigen (in this case avian influenza virus surface protein hemagglutinin).

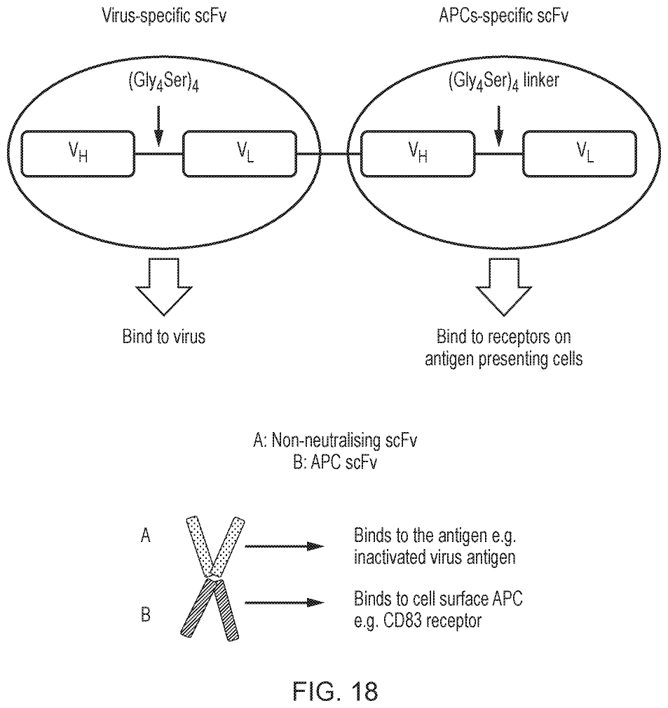

The virus specific scFv (FIG. 18) is capable of binding to virus antigen and is non-neutralising meaning that it will bind to the virus but will not neutralise it (in this example the first binding domain binds to hemagglutinin on an inactivated virus).

The APC-specific scFv (FIG. 18) is capable of binding to a cell surface protein on APCs in this example the binding domain is a CD83 scFv, which will target avian host APCs.

To demonstrate the approach of linking inactivated whole avian influenza virus antigen to the APCs cell surface receptors, a non-neutralising scFv antibody was generated (referred to herein as IG10) that specifically binds to hemagglutinin antigen of H9N2 avian influenza virus.

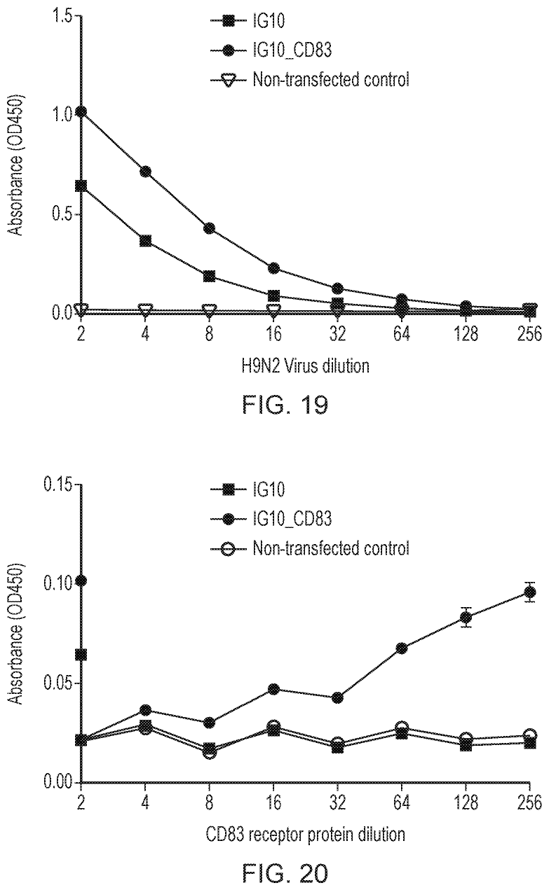

The scFv sequence of IG10 scFv was fused with CD83 scFv using a linker sequence. The resulting construct IG10 scFv-CD83 scFv was expressed as a bispecific scFv antibody in insect S2 cells and was secreted as soluble antibody (IG10 scFv-CD83 scFv) into the S2 cell culture medium. The culture medium containing secreted IG10 scFv-CD83 scFv bound specifically to inactivated H9N2 virus. The antibody construct showed specific binding to avian influenza virus and specific binding to CD83 receptor antigen in ELISA assays. The results for IG10 are shown in FIG. 19 and FIG. 20.

The results in FIG. 19 show that IG10-CD83 bispecific antibody can bind to the H9N2 virus with the affinity similar to that of IG10 scFv.

The results in FIG. 20 show that IG10-CD83 can bind and recognise chicken CD83 receptor protein. The signal for both IG10-CD83 may be considered relatively low because the proteins were not purified for this assay. It is expected that the signals will be higher with the purified bispecific antibodies.

Results from FIG. 19 and FIG. 20 show that the IG10scFv-CD83 scFv bispecific antibody has bispecific binding ability and can bind to both the H9N2 virus and chicken CD83 receptor protein.

### Example 4—Antibody Targeted Vaccine Comprising Bispecific or Multispecific Binding Domains (HD8)

To demonstrate the approach of linking inactivated whole avian influenza virus antigen to the APCs cell surface receptors, a non-neutralising scFv antibody was generated (referred to herein as HD8) that specifically binds to hemagglutinin antigen of H9N2 avian influenza virus.

The scFv sequence of HD8 scFv was fused with CD83 scFv using a linker sequence. The resulting construct HD8 scFv-CD83 scFv was expressed as a bispecific scFv antibody in insect S2 cells and was secreted as soluble antibody (HD8 scFv-CD83 scFv) into the S2 cell culture medium. The culture medium containing secreted IG10 scFv-D83 scFv bound specifically to inactivated H9N2 virus.

The binding activity of the HD8 scFv-CD83 scFv is tested by ELISA as described in Example 3.

### Example 5—Vaccine Formulation Bispecific Antibodies

During vaccine formulation bispecific or multispecific antibodies are mixed with an inactivated virus (such as a commercial killed virus vaccine formulation). This will result in the formation of antibody conjugated with the inactivated virus. In this way the antigen of inactivated virus targets the APCs. This vaccine formulation enhances the efficacy of the inactivated vaccine by APCs without the need for any chemical conjugation.

### Example 6—Immunization with H5HA Fused with CD83 scFv Protein Induces Faster and Higher Humoral Response

### Construction of H5HA and H5HA-CD83scFv Expression Plasmids.

H5HA vaccine constructs (H5HA-Foldon-CD83scFv and H5HA-Foldon) were generated using the same method that describes the generation of H9HA expression cassettes H9HA-Foldon-CD83scFv (FIG. 1) and H9HA-Foldon (FIG. 2). The expression cassette (BIP-H5HA-Foldon-CD83scFv-Ctag, SEQ ID NO: 72) contained an ectodomain sequence (amino acid 17-527) of H5HA antigen of avian influenza H5N8 virus strain (A/duck/Egypt/SS19/2017, accession no. MH893738.1). The H5HA sequence was modified to change HA cleavage site from polybasic to monobasic, the hemagglutinin gene signal peptide was replaced by Drosophila BiP protein signal sequence and the TM domain, replaced with a 30 amino acid trimerization foldon sequence of the trimeric protein fibritin from bacteriophage T4. Four amino acid “EPEA” sequence as tag (termed as EPEA tag or Ctag) was fused at the c-terminus of the expression cassette that serve for recombinant protein expression detection and purification. The second expression cassette (BIP-H5HA-Foldon-Ctag, SEQ ID NO: 73) lacks the CD83scFv sequence. Both expression cassettes were cloned into Drosophila melanogasterexpression vector (pS2V1) using EcoR1 and SaclI cloning sites.

Expression and Purification of H5HA and H5HA Fused scFv Proteins

Recombinant proteins were produced and purified using the Drosophila Expression System (DES®, Life technologies). Briefly, pExpreS2-V1 plasmids containing expression cassettes (BIP-H5HA-Foldon-CD83scFv-Ctag or BIP-H5HA-Foldon-Ctag) were transfected into Drosophila S2 cells. Antibiotic selection was carried out for four weeks using Zeocine at a concentration of 1.5 mg/mL Zeocin. The Zeocine selected cells were cultured at 28° C. in serum free media (EX-CELL©, Merck). Recombinant proteins were secreted into culture supernatant and then purified using Ctag Affinity Matrix (CaptureSelect™, Thermo Fisher). Concentration of purified recombinant proteins was determined by Bradford assay and the purity was assessed by standard SDS-PAGE and Western Blot.

### Chicken Vaccination

Groups (N=4 per groups) of one-day-old white leghorn SPF chickens were immunized with vaccine containing equimolar concentration of H5HA protein in H5HA-Foldon-CD83scFv and H5HA-Foldon vaccines. The proteins were formulated in Montanide™ ISA 71 VG (Seppic) adjuvant. The ratio of protein to adjuvant volume was 1:3. The vaccine dose (0.2 ml) was administered subcutaneous, delivered at the back of the neck. Blood samples were collected from the wing vein on 7, 14, 21 28, and 35 days of age and serum was analysed for the presence of H5HA specific antibodies using HI assays.

### Results:

### Production of H5HA-Foldon-CD83scFv and H5HA-Foldon Vaccines

The expression levels of recombinant H5HA-Foldon-CD83scFv and H5HA-Foldon proteins were in the range from 90-120 mg/litre of culture supernatants. The purity of proteins visualised using SDS-PAGE analysis that showed single band of monomeric proteins with molecular weight of 100 kDa of H5HA-Foldon-CD83scFv and 70 kDa of H5HA-Foldon. The purity of both proteins was estimated up to 99%.

Immunization with H5HA Foldon-CD83scFv Vaccine Induces Faster and Higher Humoral Response

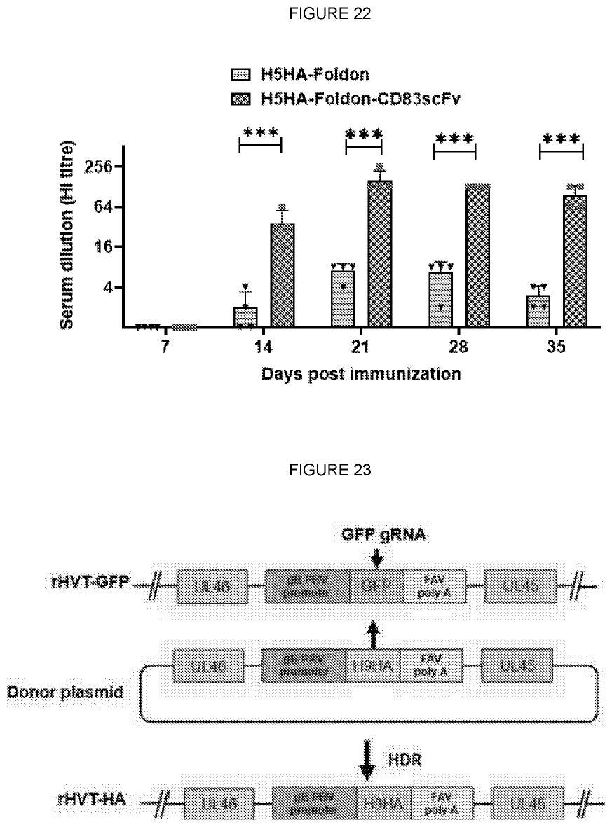

One-day-old chicks were vaccinated with 0.2 mL per dose of vaccine containing equimolar concentration of purified H5HA-Foldon-CD83scFv (49 μg) and H5HA-Foldon (35 μg). Serum samples collected at 7, 14, 21 28, and 35 days of age were analysed using standard HI assay against the inactivated virus antigens (A/Duck/Egypt/SS19/2017). The data present in FIG. 22 show that the chickens vaccinated with H5HA-Foldon-CD83scFv contained markedly higher levels of HI titres compared with chickens vaccinated with H5HA-Foldon. Conclusion: The AIV H5HA fused with CD83scFv antibody induced significantly faster and higher immune responses compared with the AIV H5HA that lacks CD83scFv antibody.

### Example 7—Recombinant Herpesvirus of Turkey (rHVT) Expressing H9HA-Foldon-CD83scFv Proteins Induces Faster and Higher Humoral Response in Chickens

### Results:

Generation of rHVT-H9HA-Foldon-CD83scFv and rHVT-H9HA-Foldon Using HDR CRISPR/Cas9 System.

The rHVT-H9HA-Foldon vaccine and rHVT-H9HA-Foldon-CD83scFv vaccine was generated using HDR-CRISPR/Cas9 is illustrated in FIG. 23. The expression cassette that produce H9HA-Foldon and H9HA-Foldon-CD83scFv proteins was integrated into the intergenic region of HVT genome between UL45/UL46 contained glycoprotein B (gB) promoter from pseudorabies virus (PRV) and polyA terminator of felid alpha herpesvirus 1. The HA protein sequence was derived from H9N2 virus strain A/chicken/Pakistan/SKP/2016 (Genbank accession number: AVX19091.1).

To rescue the rHVT, CEF cells were transfected with GFP gRNA plasmid and each of the donor plasmid containing expression cassettes (H9HA-Foldon vaccine and rHVT-H9HA-Foldon-CD83scFv) which were flanked by sequences homologous to the Cas9 cut sites. This was followed by infection with rHVT-GFP at a multiplicity of infection (MOI) of 0.01 at 12 hours post transfection. The rHVT virus plaques containing expression cassette of H9HA-Foldon or rHVT-H9HA-Foldon-CD83scFv were identified H9HA-Foldon vaccine and rHVT-H9HA-Foldon-CD83 scFv which were negative for green fluorescence. These GFP negative plaques are either correct rHVT-H9HA positive clones or false positive clones with silenced GFP. Viral DNA was extracted and subjected to PCR analysis using primers targeting the region within H9HA insert. In total, 11% and 22% of the clones were positive for H9HA-Foldon and H9HA-Foldon-CD83scFv insertions, respectively. One of the positive rHVT clone from each construct was taken forward for plaque purification, vaccine stock preparation, in vitro replication kinetics, insert stability and evaluation of immunogenicity in chickens.

The rHVT-H9HA-Foldon-CD83scFv and rHVT-H9HA-Foldon Show Similar in Vitro Replication Kinetics as that of the Wild-Type HVT.

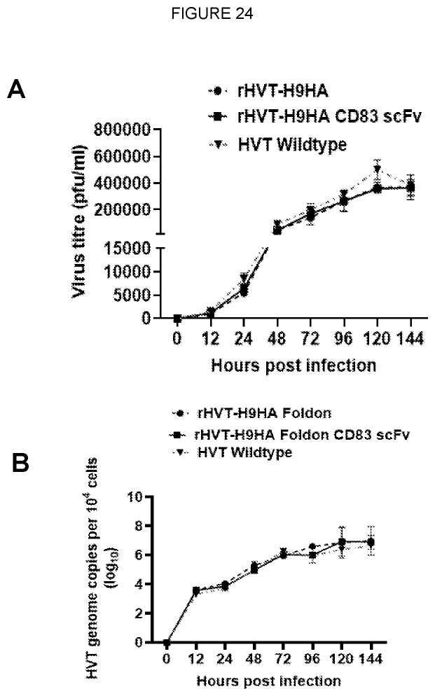

The replication fitness of rHVT-H9HA-Foldon-CD83scfv was compared with the wild-type HVT to determine whether insertion of expression cassettes (H9HA-Foldon or H9HA-Foldon-CD83scFv) affected the infectivity and replication of rHVT constructs in cultured cells. For this, chicken embryo fibroblast (CEF) cells were infected with 100 pfu of either HVT wild-type, rHVT-H9HA or rHVT-H9HA-CD83scFv. Virus replication rates were measured by counting plaque (FIG. 24A) and qRT-PCR for genome copy numbers (FIG. 24B). The rate of virus replication measured by plaque assays or qRT-PCR showed no differences in virus replication fitness between the wild-type HVT and the rHVT-H9HA-Foldon and rHVT-H9HA-Foldon-CD83scFv vaccine constructs.

The rHVT-H9HA-Foldon-CD83scFv Induces Higher Antibody Responses Compared to rHVT-H9HA-Foldon in Vaccinated Chickens.

Groups of one-day-old White Leghorn SPF chickens (n=20 per group) were immunised with 4000 pfu of rHVT-H9HA-Foldon and rHVT-H9HA-Foldon-CD83scFv, subcutaneously. Blood samples were collected from the wing veins on day 6, 14, 21, 28 and 35 pv and serum samples were subjected to HI, anti-HA IgY ELISA and virus MNT assays to measure the HA antigen-specific antibody titres.

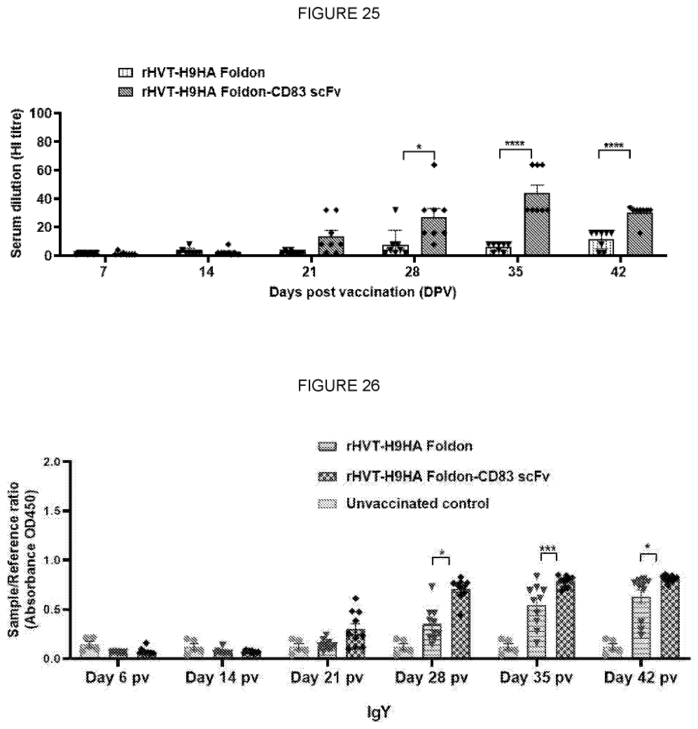

The chickens vaccinated with rHVT-H9HA-Foldon-CD83scFv vaccine showed detectable HI antibodies on day 21 pv whereas those group of chickens vaccinated with rHVT-H9HA-Foldon showed detectable levels of HI antibodies only from day 28 pv. (FIG. 25).

Comparison of HI antibody titres between rHVT-H9HA-Foldon-CD83scFv and rHVT-H9HA-Foldon demonstrated that the rHVT-H9HA-Foldon-CD83scFv was able to induce significantly higher HI antibody titres than the rHVT-H9HA-Foldon after day 21 pv (day 28 pv: p<0.05, day 35 pv: p<0.0001, day 42 pv: p<0.0001) (FIG. 25).

Analysis of IgY antibodies titres in serum samples collected from vaccinated chickens at different time points (day 6, 14, 21, 28 and 35 pv) also showed that rHVT-H9HA-Foldon-CD83scFv vaccine induced higher anti-H9HA IgY antibody titres compared to the rHVT-H9HA-Foldon vaccine (FIG. 26). The rHVT-H9HA-Foldon-CD83scFv group showed significantly higher anti-HA IgY antibodies compared to rHVT-H9HA-Foldon group on day 21 pv (p<0.05), day 28 pv (p<0.001) and day 35 pv (p<0.05).

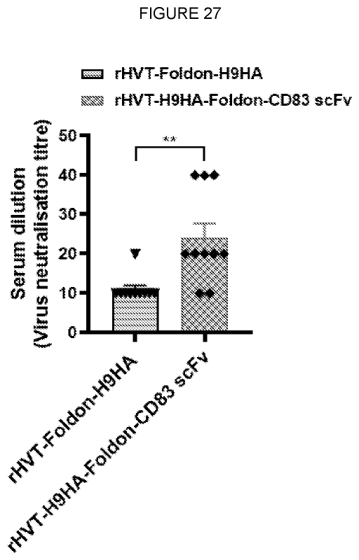

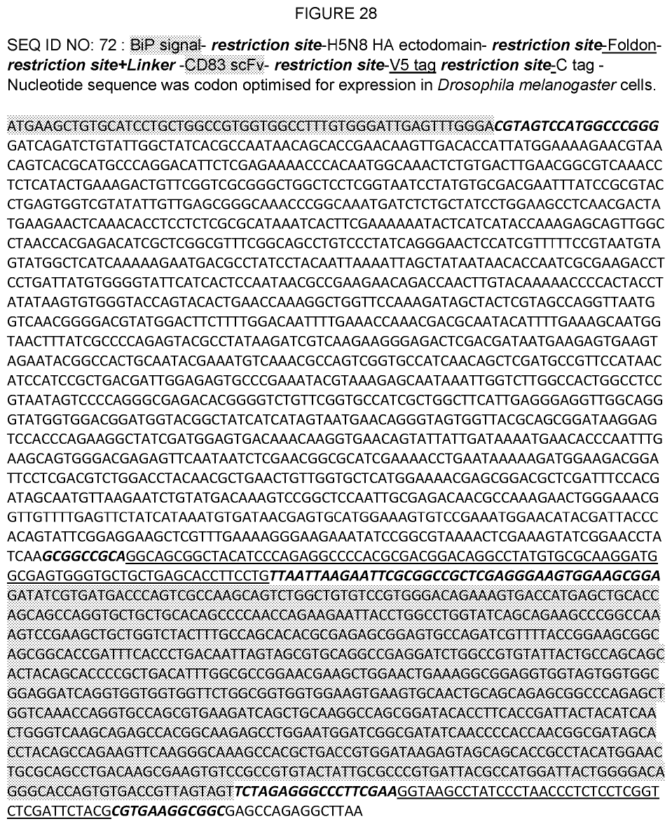

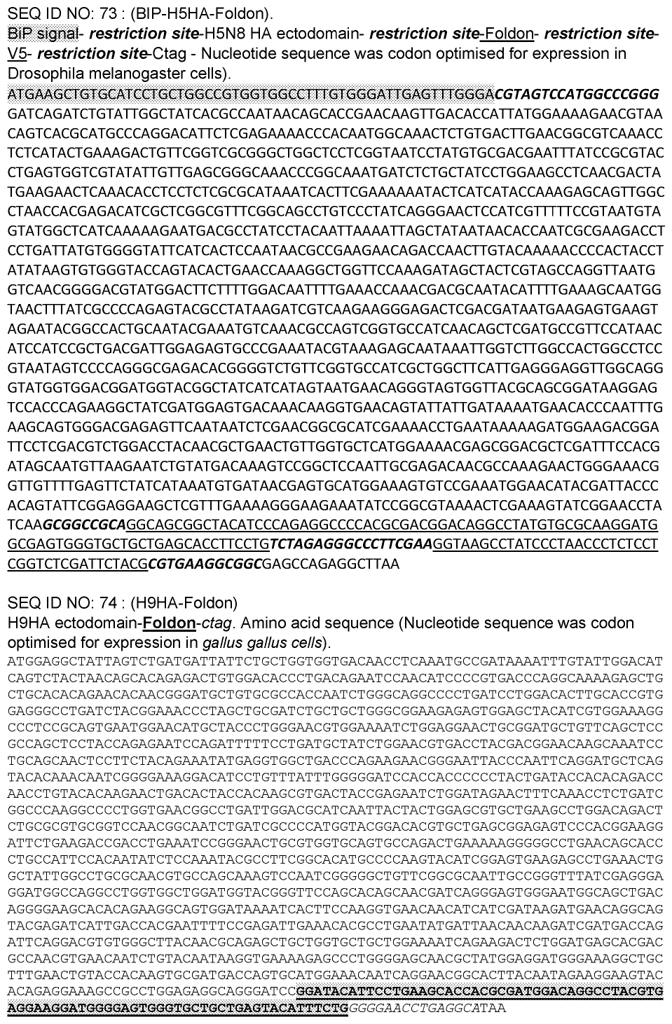

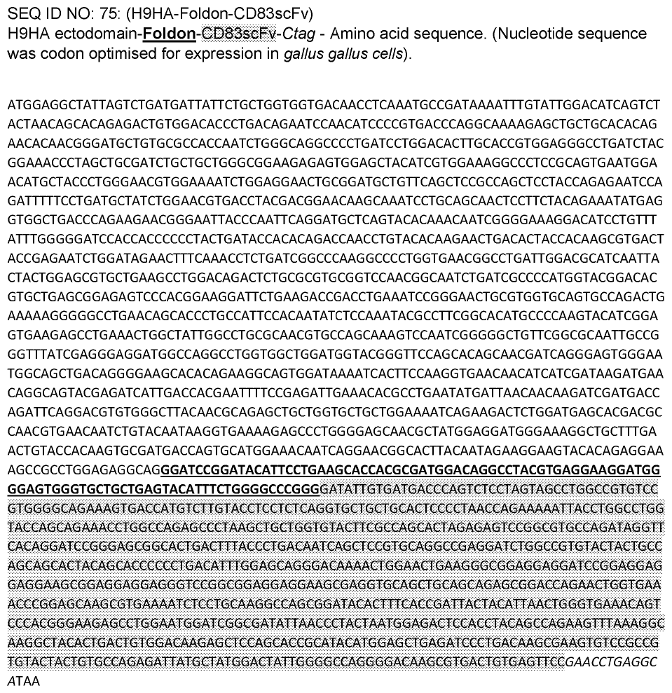

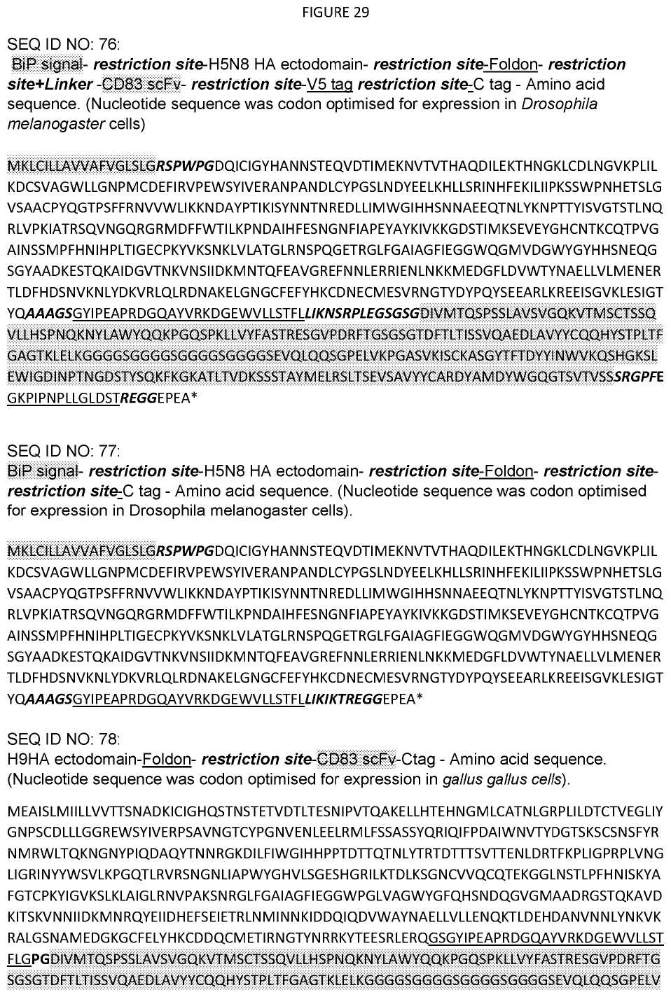

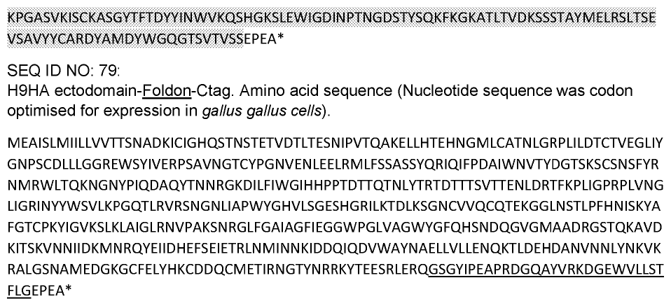

The analysis of serum antibodies levels that specifically neutralise the H9N2 virus were determined using micro-neutralisation (MNT) assay. The serum samples collected on day 42 pv from chickens vaccinated with rHVT-H9HA-Foldon-CD83scFv showed significantly higher levels virus neutralising antibodies compared to rHVT-H9HA-Foldon (p<0.01) (FIG. 27).

### Example 8—Generation of Recombinant Newcastle Disease Virus (rNDV) Expressing H9HA-Foldon-CD83scFv

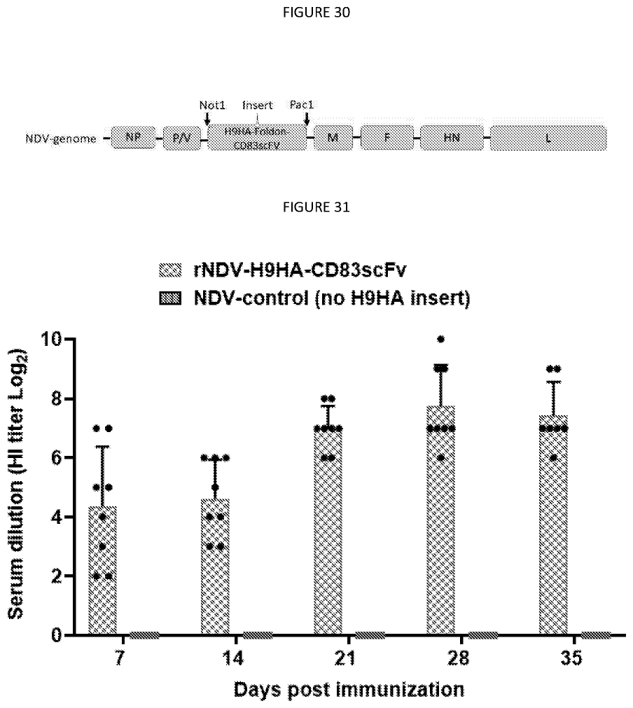

A rNDV was generated carrying an expression cassette (H9HA-Foldon-CD83scFv) that encodes a soluble form of trimeric H9HA antigen fused with scFv antibody specific for chicken APCs receptor, CD83. The expression cassette (SEQ ID NO: 75) was chemically synthesised, and codon optimised for expression in chicken (Gallus gallus) cells (GenScript). The expression cassette was integrated in the NDV Intergenic region (between P/V and M genes) of LaSota strain (FIG. 30). The generated rNDV vaccine constructs produced secreted forms of H9HA-Foldon-CD83scFv antigens. Vaccination of 7-day-old chickens with the rNDV-H9HA-Foldon-CD83scFv vaccine elicited strong H9HA-antigen-specific HI antibody titres (FIG. 31). The results conclude that NDV can be used as a vector for the production and delivery of APCs targeting vaccines in chicken.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. An engineered protein comprising: at least one binding domain which is capable of binding to a cell surface protein on an avian antigen presenting cell; and
a) at least one antigenic polypeptide; or
b) at least one binding domain which is capable of binding to at least one antigenic polypeptide.

2. An engineered protein according to claim 1, wherein said engineered protein is a genetically engineered protein.

3. An engineered protein according to claim 2, wherein the at least one binding domain capable of binding to a cell surface protein and the at least one antigenic polypeptide are comprised in a single recombinant protein; or the at least one binding domain capable of binding to a cell surface protein and the at least one binding domain which is capable of binding to at least one antigenic polypeptide are comprised in a single recombinant protein.

4. An engineered protein according to claim 1, wherein the at least one binding domain capable of binding to a cell surface protein is operably linked to the at least one antigenic polypeptide or the at least one binding domain which is capable of binding to an antigenic polypeptide.

5. An engineered protein according to claim 1, wherein the antigen presenting cell is at least one of a dendritic cell, macrophage, B cell or natural killer cell.

6. An engineered protein according to claim 1, wherein the cell surface protein is selected from an immunoglobulin family protein, an integrin family receptor or a C-type lectin.

7. An engineered protein according to claim 1, wherein the cell surface protein is selected from CD83, CD11c or Dec205.

8. An engineered protein according to claim 1, wherein the cell surface protein is CD83.

9. An engineered protein according to claim 1, wherein the at least one antigenic polypeptide is an avian influenza virus antigenic polypeptide, such as hemagglutinin.

10. An engineered protein according to claim 1, wherein the binding domain(s) is (are) based on the antigen binding site of an antibody or an antibody fragment such as a single-chain variable fragment (scFv), Fv, F(ab′) or F(ab′)2.

11. A nucleic acid construct which comprises a first polynucleotide which encodes at least one binding domain which is capable of binding to a cell surface protein on an avian antigen presenting cell as defined in claim 1; and a second polynucleotide which encodes at least one antigenic polypeptide or at least one binding domain which is capable of binding to at least one antigenic polypeptide as defined in claim 1.

12. A vector which comprises a nucleic acid construct according to claim 11.

13. An engineered cell comprising a nucleic acid construct according to claim 11.

14. An avian vaccine comprising an engineered protein according to claim 1.

15. A vaccine according to claim 14 for use in treating and/or preventing disease in an avian subject.

16. (canceled)

17. A method for treating and/or preventing a disease in an avian subject which comprises the step of administering to a subject an effective amount of a vaccine according to claim 14.

18. A method according to claim 17, wherein administration of said vaccine elicits a humoral and/or cellular immune response in the subject.

19. A method according to claim 17, wherein administration of said vaccine decreases the challenge pathogen load in the subject.

20. A method according to claim 17, wherein the subject is poultry, optionally a chicken, turkey, duck, quail, pigeon or goose.

21. A method for the preparation of the vaccine according to claim 14, the method comprising the step of admixing a genetically engineered protein according to claim 1, and a pharmaceutically acceptable carrier.
